(19) United States
(12) Patent Application Publication
(10) Pub. No.: US 2009/0054453 A1
(43) Pub. Date: Feb. 26, 2009

(54) Novel Tetralins as 5-HT6 Modulators

(76) Inventors: Lilian Alcaraz, Leicestershire (GB); Gunnar Nordvall, Sodertalje (SE); Didier Rotticci, Sodertalje (SE); Daniel Sohn, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 12/293,176

(22) PCT Filed: Mar. 15, 2007
(86) PCT No.: PCT/SE2007/000252

§ 371 Date: Sep. 16, 2008

Related U.S. Application Data
(60) Provisional application No. 60/783,576, filed on Mar. 17, 2006.

(51) Int. Cl. A61K 31/496; C07D 241/04; C07D 413/02; C07D 403/02; A61K 31/495

(52) U.S. Cl. 514/253.05; 544/392; 514/255.03; 514/254.04; 544/369; 544/363

(57) ABSTRACT

The present invention relates to new compounds of formula I, or salts, solvates or solvated salts thereof, wherein Q, R¹, R², R³, R⁴ and n are defined as in claim 1, processes for their preparation and to new intermediates used in the preparation thereof, pharmaceutical formulations containing said compounds and to the use of said compounds in therapy.

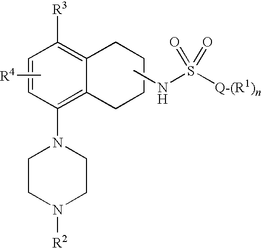

### FIELD OF THE INVENTION

The present invention relates to new compounds, to pharmaceutical formulations containing said compounds and to the use of said compounds in therapy. The present invention further relates to processes for the preparation of said compounds and to new intermediates useful in the preparation thereof.

### BACKGROUND OF THE INVENTION

Serotonin (5-hydroxy-tryptamine) (5-HT) receptors play an important role in many physiological and pathological functions like anxiety, sleep regulation, aggression, feeding and depression. The 5-HT receptors are distributed throughout the body and can be divided into seven different 5-HT receptor subtypes, i.e. 5-HT1, 5-HT2, 5-HT3, 5-HT4, 5-HT5, 5-HT6, and 5-HT7, with different properties.

The 5-HT6 receptor is mostly found in the central nervous system (CNS). From in situ hybridization studies it is known that the 5-HT6 receptor in rat brain is localized in areas like striatum, nucleus accumbens, olfactory tubercle and hippocampal formation (Ward et al., Neuroscience, 64, p 1105-1111, 1995).

Scientific research has revealed a potential therapeutic use for modulators of the 5-HT6 receptor, especially with regard to various CNS disorders. Blocking 5-HT6 receptor function has been shown to enhance cholinergic transmission (Bentley et al, Br J Pharmacol 126: 1537-1542).

Studies have shown that 5-HT6 antagonists increase levels of glutamate and aspartate in the frontal cortex and dorsal hippocampus as well as acetylcholine in the frontal cortex. These neurochemicals are known to be involved in memory and cognition (Dawson et al., Neuropsychopharmacology., 25(5), p 662-668, 2001) (Gerard et al., Brain Res., 746, p 207-219, 1997) (Riemer et al J Med Chem 46(7), p 1273-1276, 2003).

Acetylcholinesterase inhibitors increase the levels of acetylcholine in the CNS and are used in the treatment of cognitive disorders such as Alzheimer's disease. 5-HT6 antagonists may therefore be used in the treatment of cognitive disorders.

Studies have also shown that 5-HT6 antagonist increases the level of dopamine and noradrenaline in the medial prefrontal cortex suggesting a role for 5-HT6 ligands in cognitive aspects of schizophrenia. Several antidepressants and atypical antipsychotics bind to the 5-HT6 receptor and this may be a factor in their profile of activities (Roth et al., J. Pharm. Exp. Therapeut., 268, 1402-1420, 1994; Sleight et al., Exp. Opin. Ther. Patents, 8, 1217-1224, 1998; Kohen et al., J. Neurochem., 66(1), p 47-56, 1996; Sleight et al. Brit. J. Pharmacol., 124, p 556-562, 1998; Bourson et al., Brit. J. Pharmacol., 125, p 1562-1566, 1998).

Stean et al., (Brit. J. Pharmacol. 127 Proc. Supplement 131P, 1999) have described the potential use of 5-HT6 modulators in the treatment of epilepsy. 5-HT6 receptors have also been linked to generalized stress and anxiety states (Yoshioka et al., Life Sciences, 17/18, p 1473-1477, 1998). The use of modulators for this receptor is therefore expected for a wide range of CNS disorders.

Moreover, a reduction in food intake in rats has been reported using 5-HT6 receptor modulators (Bentley et al., Br. J. Pharmacol. Suppl. 126, P66, 1999; Bentley et al. J. Psychopharmacol. Supl. A64, 255, 1997). 5-HT6 receptor modulators may therefore also be useful in the treatment of feeding disorders like anorexia, obesity, bulimia and similar disorders and also type 2 diabetes.

### DETAILED DESCRIPTION OF THE INVENTION

The object of the present invention is to provide compounds exhibiting a modulating activity at the 5-hydroxy-tryptamine 6 (5-HT6) receptor.

The present invention relates to a compound having the formula I, wherein:

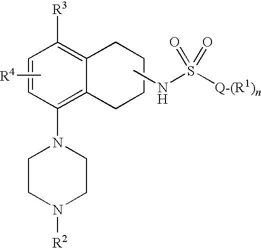

Q is selected from C₆₋₁₀alrylC₀₋₆alkyl, C₅₋₁₁heteroarylC₀₋₆alkyl, C₃₋₈cycloalkylC₀₋₆alkyl, C₃₋₈heterocycloalkylC₀₋₆alkyl, C₂₋₆alkenyl and C₂₋₁₀alkyl;
R¹ is selected from hydrogen, hydroxy, halogen, C₁₋₁₀alkyl, C₂₋₁₀alkenyl, C₂₋₁₀alkynyl, C₁₋₁₀alkoxy, N(R⁵)₂, C₆₋₁₀arylC₀₋₆alkyl, C₅₋₁₁heteroarylC₀₋₆alkyl, C₁₋₆haloalkyl, R⁵OC₀₋₆alkyl, cyano, SR⁵, R⁶SO₂C₀₋₆alkyl, SOR⁶, R⁵CON(R⁵)C₀₋₆alkyl, N(R⁵)SO₂R⁵, COR⁶, R⁵CO₂C₀₋₆alkyl, R⁵OC(O)C₀₋₆alkyl, OSO₂R⁵, C₃₋₈cycloalkyl, C₃₋₈heterocycloalkyl, (R⁵)₂NCOC₀₋₆alkyl, SO₂N(R)₂, N(R⁵)CON(R)₂, N(R⁷)COR⁸, NO₂, OR⁵ and oxo;
n is 0, 1, 2, 3 or 4;
R² is selected from hydrogen, C₁₋₆alkyl, R⁷OC₂₋₆alkyl, C₁₋₆haloalkyl, cyanoC₁₋₆alkyl, (R⁷)₂NCOC₁₋₆alkyl and R⁷CON(R⁷)C₁₋₆alkyl;
R³ is selected from hydrogen, C₁₋₆alkyl, halogen, cyano, C₁₋₆alkoxy, R⁷OC₀₋₆alkyl, C₁₋₆haloalkyl, cyanoC₁₋₆alkyl, NO₂, (R⁷)₂NCOC₀₋₃alkyl or R⁷CON(R⁷)C₀₋₃alkyl;
R⁴ is selected from hydrogen, C₁₋₆alkyl, halogen, cyano, C₁₋₆alkoxy, R⁷OC₀₋₆alkyl, C₁₋₆haloalkyl, cyanoC₁₋₆alkyl, NO₂, (R⁷)₂NCOC₀₋₃alkyl and R⁷CON(R⁷)C₀₋₃alkyl;
R⁵ is selected from hydrogen, C₁₋₁₀alkyl, C₁₋₆haloalkyl, C₃₋₈cycloalkylC₀₋₆alkyl, C₃₋₈heterocycloalkylC₀₋₆alkyl, C₆₋₁₀arylC₀₋₆alkyl and C₅₋₆heteroarylC₀₋₆alkyl;
R⁶ is selected from C₁₋₁₀alkyl, C₁₋₆haloalkyl, C₁₋₆alkoxy, C₃₋₈cycloalkylC₀₋₃alkyl, C₃₋₈heterocycloalkylC₀₋₆alkyl, C₆₋₁₀arylC₀₋₃alkyl and C₅₋₆heteroarylC₀₋₃alkyl; and
wherein any aryl, cycloalkyl, heterocycloalkyl or heteroaryl as defined for R¹, R⁵ and R⁶ may be substituted by one or more groups independently selected from hydrogen, halogen, hydroxy, C₁₋₃haloalkyl, cyano, OR⁸, C₁₋₆alkyl, oxo, CON(R⁷)₂, N(R⁷)COR⁸, SO₂R⁸, SOR⁸, N(R⁷)₂ and COR⁷;
R⁷ is hydrogen, C₁₋₆alkyl or C₁₋₆haloalkyl; and
R⁸ is C₁₋₆alkyl or C₁₋₆haloalkyl;
or pharmaceutically acceptable salts, solvates or solvated salts thereof.

One embodiment of the present invention relates to a compound of formula I, wherein:

Q is selected from C₆₋₁₀arylC₀₋₆alkyl, C₅₋₁₁heteroarylC₀₋₆alkyl, C₃₋₈cycloalkylC₀₋₆alkyl, C₃₋₈heterocycloalkylC₀₋₆alkyl and C₂₋₁₀alkyl;
R¹ is selected from hydrogen, halogen, C₁₋₁₀alkyl, C₁₋₁₀alkoxy, N(R⁵)₂, C₆₋₁₀arylC₀₋₆alkyl, C₅₋₁₁heteroarylC₀₋₆alkyl, C₁₋₆haloalkyl, R⁵OC₀₋₆alkyl, cyano, SR⁵, R⁶SO₂C₀₋₆alkyl, N(R⁵)SO₂R⁵, COR⁶, R⁵CO₂C₀₋₆alkyl, R⁵OC(O)C₀₋₆alkyl, OSO₂R⁵, C₃₋₈cycloalkyl, C₃₋₈heterocycloalkyl, (R⁵)₂NCOC₀₋₆alkyl, SO₂N(R⁵)₂, N(R⁵)CON(R⁵)₂, N(R⁷)COR⁸, NO₂, OR⁵ and oxo;
n is 0, 1, 2, 3 or 4;
R² is selected from hydrogen, C₁₋₆alkyl, R⁷OC₂₋₆alkyl and C₁₋₆haloalkyl;
R³ is selected from hydrogen, C₁₋₆alkyl, halogen cyano and C₁₋₆alkoxy;
R⁴ is selected from hydrogen, C₁₋₆alkyl, halogen, cyano, C₁₋₆alkoxy, R⁷OC₀₋₆alkyl, C₁₋₆haloalkyl, cyanoC₁₋₆alkyl, NO₂, (R⁷)₂NCOC₀₋₃alkyl and R⁷CON(R⁷)C₀₋₃alkyl;
R⁵ is selected from hydrogen, C₁₋₁₀alkyl, C₁₋₆haloalkyl, C₃₋₈cycloalkylC₀₋₆alkyl, C₃₋₈heterocycloalkylC₀₋₆alkyl and C₆₋₁₀arylC₀₋₆alkyl;
R⁶ is selected from C₁₋₁₀alkyl, C₁₋₆haloalkyl, C₁₋₆alkoxy, C₃₋₈cycloalkylC₀₋₃alkyl and C₃₋₈heterocycloalkylC₀₋₆alkyl; and
wherein any aryl, cycloalkyl, heterocycloalkyl or heteroaryl as defined for R¹, R⁵ and R⁶ may be substituted by one or more groups independently selected from hydrogen, halogen, hydroxy, C₁₋₃haloalkyl, cyano, OR⁸, C₁₋₆alkyl, oxo, CON(R⁷)₂, N(R⁷)COR⁸, SO₂R⁸, SOR⁸, N(R⁷)₂ and COR⁷;
R⁷ is hydrogen, C₁₋₆alkyl or C₁₋₆haloalkyl; and
R⁸ is C₁₋₆alkyl or C₁₋₆haloalkyl;
or pharmaceutically acceptable salts, solvates or solvated salts thereof.

Another embodiment of the present invention relates to a compound of formula I, wherein:

Q is selected from C₆₋₁₀arylC₀₋₆alkyl, C₅₋₁₁heteroarylC₀₋₆alkyl, C₃₋₈cycloalkylC₀₋₆alkyl and C₃₋₈heterocycloalkylC₀₋₆alkyl;
R¹ is selected from hydrogen, halogen, C₁₋₁₀alkyl, C₁₋₁₀alkoxy, N(R⁵)₂, C₆₋₁₀arylC₀₋₆alkyl, C₅₋₁₁heteroarylC₀₋₆alkyl, C₁₋₆haloalkyl, R⁵OC₀₋₆alkyl, cyano, SR⁵, R⁶SO₂C₀₋₆alkyl, N(R⁵)SO₂R⁵, COR⁶, R⁵CO₂C₀₋₆alkyl, R⁵OC(O)C₀₋₆alkyl, OSO₂R⁵, C₃₋₈cycloalkyl, C₃₋₈heterocycloalkyl, (R⁵)₂NCOC₀₋₆alkyl, SO₂N(R⁵)₂, N(R)CON(R⁵)₂, N(R⁷)COR⁸, NO₂, OR⁵ and oxo;
n is 0, 1, 2, 3 or 4;
R² is selected from hydrogen, C₁₋₆alkyl and C₁₋₆haloalkyl;
R³ is selected from hydrogen, C₁₋₆alkyl, halogen and C₁₋₆alkoxy;
R⁴ is selected from hydrogen, C₁₋₆alkyl, halogen, cyano and C₁₋₆alkoxy;
R⁵ is selected from hydrogen, C₁₋₁₀alkyl, C₃₋₈cycloalkylC₀₋₆alkyl, C₃₋₈heterocycloalkylC₀₋₆alkyl and C₆₋₁₀arylC₀₋₆alkyl;
R⁶ is selected from C₁₋₁₀alkyl, C₁₋₆haloalkyl and C₁₋₆alkoxy; and
wherein any aryl, cycloalkyl, heterocycloalkyl or heteroaryl as defined for R¹, R⁵ and R⁶ may be substituted by one or more groups independently selected from hydrogen, halogen, hydroxy, C₁₋₃haloalkyl, cyano, OR⁸, C₁₋₆alkyl, oxo, CON(R⁷)₂, N(R⁷)COR⁸, SO₂R⁸, SOR⁸, N(R⁷)₂ and COR⁷;
R⁷ is hydrogen; and
R⁸ is C₁₋₆alkyl;
or pharmaceutically acceptable salts, solvates or solvated salts thereof.

A further embodiment of the present invention of the present invention relates to a The compound of formula I, wherein:

Q is selected from C₆₋₁₀arylC₀₋₆alkyl, C₅₋₁₁heteroarylC₀₋₆alkyl, C₃₋₈cycloalkylC₀₋₆alkyl and C₃₋₈heterocycloalkylC₀₋₆alkyl;
R¹ is selected from hydrogen, halogen, C₁₋₁₀alkyl, C₁₋₁₀alkoxy, C₅₋₁₁heteroarylC₀₋₆alkyl, C₁₋₆haloalkyl, R⁵OC₀₋₆alkyl, cyano, SR⁵, R⁶SO₂C₀₋₆alkyl, COR⁶, R⁵CO₂C₀₋₆alkyl, R⁵OC(O)C₀₋₆alkyl, SO₂N(R⁵)₂, N(R⁷)COR⁸, NO₂, OR⁵ and oxo;
n is 0, 1, 2 or 3;
R² is hydrogen or C₁₋₆alkyl;
R³ is hydrogen, C₁₋₆alkyl or C₁₋₆alkoxy;
R⁴ is hydrogen, C₁₋₆alkyl or C₁₋₆alkoxy;
R⁵ is selected from hydrogen, C₁₋₁₀alkyl and C₆₋₁₀arylC₀₋₆alkyl;
R⁶ is selected from C₁₋₁₀alkyl, C₁₋₆haloalkyl and C₁₋₆alkoxy; and
wherein any aryl, cycloalkyl, heterocycloalkyl or heteroaryl as defined for R¹, R⁵ and R⁶ may be substituted by one or more groups independently selected from hydrogen, halogen, hydroxy, C₁₋₃haloalkyl, cyano, OR⁸, C₁₋₆alkyl, oxo, CON(R⁷)₂, N(R⁷)COR⁸, SO₂R⁸, SOR⁸, N(R⁷)₂ and COR⁷;
R⁷ is hydrogen; and
R⁸ is C₁₋₆alkyl;
or pharmaceutically acceptable salts, solvates or solvated salts thereof.

According to one embodiment of the present invention, Q is phenyl.

According to another embodiment of the present invention, R² is hydrogen or C₁₋₃alkyl; R³ is hydrogen, C₁₋₃alkyl or C₁₋₃alkoxy; and R⁴ is hydrogen, C₁₋₃alkyl or C₁₋₃alkoxy.

According to yet another embodiment of the present invention, Q is selected from C₆₋₁₀arylC₀₋₃alkyl, C₅₋₁₁heteroarylC₀₋₃alkyl, C₃₋₈cycloalkylC₀₋₃alkyl or C₂₋₄alkenyl and C₂₋₅alkyl;

R¹ is selected from hydrogen, halogen, C₁₋₄alkyl, Cl₄alkoxy, C₅₋₁₁heteroarylC₀₋₃alkyl, C₁₋₆haloalkyl, R⁵OC₀₋₃alkyl, cyano, R⁶SO₂C₀₋₃alkyl, R⁵CON(R⁵)C₀₋₃alkyl, R⁵OC(O)C₀₋₆alkyl, (R⁵)₂NCOC₀₋₃alkyl, SO₂N(R⁵)₂, NO₂ and oxo;
n is 0, 1, 2, 3 or 4;
R² is hydrogen or C₁₋₃alkyl;
R³ is hydrogen, C₁₋₃alkyl or C₁₋₃alkoxy;
R⁴ is hydrogen;
R⁵ is hydrogen, C₁₋₃alkyl, C₁₋₃haloalkyl, C₆₋₁₀arylC₀₋₃alkyl or C₅₋₆heteroarylC₀₋₃alkyl;
R⁶ is C₁₋₄alkyl or C₆₋₁₀arylC₀₋₃alkyl; and
wherein any aryl, cycloalkyl, heterocycloalkyl or heteroaryl as defined for R¹, R⁵ and R⁶ may be substituted by one or more groups independently selected from hydrogen, halogen, and C₁₋₃haloalkyl;
or pharmaceutically acceptable salts, solvates or solvated salts thereof.

In yet another embodiment of the invention the sulphonamide-substituent is attached at position 2 or 3 of the tetrahydronaphthalen core of the compound of formula I.

In a further embodiment of the present invention, n is 0, 1, 2 or 3.

The present invention also related to compounds selected from:
3-chloro-N-[(2R)-8-methoxy-5-(4-methylpiperazin-1-yl)-1,2,3,4-tetrahydronaphthalen-2-yl]benzenesulfonamide,
4-chloro-N-[(2R)-8-methoxy-5-(4-methylpiperazin-1-yl)-1,2,3,4-tetrahydronaphthalen-2-yl]benzenesulfonamide,
3-chloro-N-[(2S)-5-(4-methylpiperazin-1-yl)-1,2,3,4-tetrahydronaphthalen-2-yl]benzenesulfonamide,
3-chloro-4-methyl-N-[(2S)-5-(4-methylpiperazin-1-yl)-1,2,3,4-tetrahydronaphthalen-2-yl]benzenesulfonamide,
5-chloro-N-[(2S)-5-(4-methylpiperazin-1-yl)-1,2,3,4-tetrahydronaphthalen-2-yl]naphthalene-2-sulfonamide,
3-chloro-4-methyl-N-[(2S)-5-piperazin-1-yl-1,2,3,4-tetrahydronaphthalen-2-yl]benzenesulfonamide,
3-chloro-N-[(2S)-5-piperazin-1-yl-1,2,3,4-tetrahydronaphthalen-2-yl]benzenesulfonamide,
3-chloro-N-[(2S)-8-methoxy-5-piperazin-1-yl-1,2,3,4-tetrahydronaphthalen-2-yl]benzenesulfonamide,
N-[(2S)-8-methoxy-5-piperazin-1-yl-1,2,3,4-tetrahydronaphthalen-2-yl]-3,5-dimethylisoxazole-4-sulfonamide,
1-[3-chloro-5-(trifluoromethyl)pyridin-2-yl]-N-[(2S)-8-methoxy-5-piperazin-1-yl-1,2,3,4-tetrahydronaphthalen-2-yl]-1H-pyrrole-2-sulfonamide,
2,3-dichloro-N-[(2R)-5-methyl-8-(4-methylpiperazin-1-yl)-1,2,3,4-tetrahydronaphthalen-2-yl]benzenesulfonamide,
N-[(2R)-8-(4-methylpiperazin-1-yl)-1,2,3,4-tetrahydronaphthalen-2-yl]naphthalene-2-sulfonamide,
3-chloro-4-methyl-N-[(2R)-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]naphthalene-2-sulfonamide,
1-[3-chloro-5-(trifluoromethyl)pyridin-2-yl]-N-[(2R)-5-methyl-8-(4-methylpiperazin-1-yl)-1,2,3,4-tetrahydronaphthalen-2-yl]-1H-pyrrole-2-sulfonamide,
5-chloro-N-[(2R)-5-methyl-8-piperazin-1-yl-1,2,3,4-tetrahydronaphthalen-2-yl]naphthalene-2-sulfonamide,
N-[(2R)-5-n-ethyl-8-piperazin-1-yl-1,2,3,4-tetrahydronaphthalen-2-yl]naphthalene-2-sulfonamide,
2,6-dichloro-N-[(2R)-5-methyl-8-piperazin-1-yl-1,2,3,4-tetrahydronaphthalen-2-yl]benzenesulfonamide,
3,5-dimethyl-N-[(2R)-5-methyl-8-piperazin-1-yl-1,2,3,4-tetrahydronaphthalen-2-yl]isoxazole-4-sulfonamide,
2-chloro-6-methyl-N-[(2R)-5-methyl-8-piperazin-1-yl-1,2,3,4-tetrahydronaphthalen-2-yl]benzenesulfonamide,
2,6-difluoro-N-[(2R)-5-methyl-8-piperazin-1-yl-1,2,3,4-tetrahydronaphthalen-2-yl]benzenesulfonamide,
N-[(2R)-5-methyl-8-piperazin-1-yl-1,2,3,4-tetrahydronaphthalen-2-yl]isoquinoline-5-sulfonamide,
5-chloro-1,3-dimethyl-N-[(2R)-5-methyl-8-piperazin-1-yl-1,2,3,4-tetrahydronaphthalen-2-yl]-11H-pyrazole-4-sulfonamide,
2,4-dimethyl-N-[(2R)-5-methyl-8-piperazin-1-yl-1,2,3,4-tetrahydronaphthalen-2-yl]-1,3-thiazole-5-sulfonamide,
1,3,5-trimethyl-N-[(2R)-5-methyl-8-piperazin-1-yl-1,2,3,4-tetrahydronaphthalen-2-yl]-1H-pyrazole-4-sulfonamide,
4-bromo-2,5-dichloro-N-[(2R)-5-methyl-8-piperazin-1-yl-1,2,3,4-tetrahydronaphthalen-2-yl]thiophene-3-sulfonamide,
4-bromo-N-[(2R)-5-methyl-g-piperazin-1-yl-1,2,3,4-tetrahydronaphthalen-2-yl]thiophene-3-sulfonamide,
N-[4-methyl-5-({[(2R)-5-methyl-8-piperazin-1-yl-1,2,3,4-tetrahydronaphthalen-2-yl]amino}sulfonyl)-1,3-thiazol-2-yl]acetamide,
2,4-dDimethyl-N-[(2R)-5-methyl-8-(4-methylpiperazin-1-yl)-1,2,3,4-tetrahydronaphthalen-2-yl]-1,3-thiazole-5-sulfonamide,
N-[(2R)-5-methyl-8-(4-methylpiperazin-1-yl)-1,2,3,4-tetrahydronaphthalen-2-yl]-1,3-benzothiazole-6-sulfonamide,
5-chloro-N-[(2R)-8-piperazin-1-yl-1,2,3,4-tetrahydronaphthalen-2-yl]naphthalene-2-sulfonamide,
4-chloro-N-[(2R)-8-piperazin-1-yl-1,2,3,4-tetrahydronaphthalen-2-yl]naphthalene-1-sulfonamide,
5-chloro-N-[(2R)-8-piperazin-1-yl-1,2,3,4-tetrahydronaphthalen-2-yl]naphthalene-1-sulfonamide,
1-(3-chlorophenyl)-N-[(2R)-8-piperazin-1-yl-1,2,3,4-tetrahydronaphthalen-2-yl]methanesulfonamide,
2-(4-chlorophenyl)-N-[(2R)-8-piperazin-1-yl-1,2,3,4-tetrahydronaphthalen-2-yl]methanesulfonamide,
N-[(2R)-5-methyl-8-(4-methylpiperazin-1-yl)-1,2,3,4-tetrahydronaphthalen-2-yl]benzenesulfonamide,
3-cyano-N-[(2R)-5-methyl-8-(4-methylpiperazin-1-yl)-1,2,3,4-tetrahydronaphthalen-2-yl]benzenesulfonamide,
5-chloro-1,3-dimethyl-N-[(2R)-5-methyl-8-(4-methylpiperazin-1-yl)-1,2,3,4-tetrahydronaphthalen-2-yl]-1H-pyrazole-4-sulfonamide,
3,5-dimethyl-N-[(2R)-5-methyl-8-(4-methylpiperazin-1-yl)-1,2,3,4-tetrahydronaphthalen-2-yl]isoxazole-4-sulfonamide,
2,6-difluoro-N-[(2R)-5-methyl-8-(4-methylpiperazin-1-yl)-1,2,3,4-tetrahydronaphthalen-2-yl]benzenesulfonamide,
2-chloro-4-fluoro-N-[(2R)-5-methyl-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]-2-(trifluoromethyl)benzenesulfonamide,
5-chloro-2-methoxy-N-[(2R)-5-methyl-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]-4-nitrobenzenesulfonamide,
N-[(2R)-5-methyl-8-(4-methylpiperazin-1-yl)-1,2,3,4-tetrahydronaphthalen-2-yl]-1-phenylmethanesulfonamide,
4-fluoro-N-[(2R)-5-methyl-8-(4-methylpiperazin-1-yl)-1,2,3,4-tetrahydronaphthalen-2-yl]benzenesulfonamide,
N-[4-({[(2R)-5-methyl-8-(4-methylpiperazin-1-yl)-1,2,3,4-tetrahydronaphthalen-2-yl]amino}sulfonyl)phenyl]acetamide,
2,5-difluoro-N-[(2R)-5-methyl-8-(4-methylpiperazin-1-yl)-1,2,3,4-tetrahydronaphthalen-2-yl]benzenesulfonamide,
methyl 1-methyl-5-({[(2R)-5-methyl-8-(4-methylpiperazin-1-yl)-1,2,3,4-tetrahydronaphthalen-2-yl]amino}sulfonyl)-1H-pyrrole-2-carboxylate,
4-chloro-2-fluoro-N-[(2R)-5-methyl-8-(4-methylpiperazin-1-yl)-1,2,3,4-tetrahydronaphthalen-2-yl]benzenesulfonamide,
3,5-dimethyl-N-[(2R)-5-methyl-g-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]pyridine-3-sulfonamide,
5-bromo-2,4-difluoro-N-[(2R)-5-methyl-8-(4-methylpiperazin-1-yl)-1,2,3,4-tetrahydronaphthalen-2-yl]benzenesulfonamide,
5-bromo-6-chloro-N-[(2R)-5-methyl-8-(4-methylpiperazin-1-yl)-1,2,3,4-tetrahydronaphthalen-2-yl]pyridine-3-sulfonamide,
N-[(2R)-5-methyl-8-(4-methylpiperazin-1-yl)-1,2,3,4-tetrahydronaphthalen-2-yl]-1-benzothiophene-3-sulfonamide,
4-bromo-2,5-difluoro-N-[(2R)-5-methyl-8-(4-methylpiperazin-1-yl)-1,2,3,4-tetrahydronaphthalen-2-yl]benzenesulfonamide,
2,3,4-trifluoro-N-[(2R)-5-methyl-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]thiophene-3-sulfonamide,
N-[(2R)-5-methyl-8-(4-methylpiperazin-1-yl)-1,2,3,4-tetrahydronaphthalen-2-yl]-4-(methylsulfonyl)benzenesulfonamide,
2-chloro-4-cyano-N-[(2R)-5-methyl-8-(4-methylpiperazin-1-yl)-1,2,3,4-tetrahydronaphthalen-2-yl]benzenesulfonamide,
6-chloro-N-[(2R)-5-methyl-8-(4-methylpiperazin-1-yl)-1,2,3,4-tetrahydronaphthalen-2-yl]imidazo[2,1-b][1,3]thiazole-5-sulfonamide,
4-chloro-N-[(2R)-5-methyl-8-(4-methylpiperazin 1-yl)-1,2,3,4-tetrahydronaphthalen-2-yl]benzenesulfonamide,
3-chloro-N-[(2R)-5-methyl-8-(4-methylpiperazin-1-yl)-1,2,3,4-tetrahydronaphthalen-2-yl]benzenesulfonamide,
1-(3-chlorophenyl)-N-[(2R)-5-methyl-8-(4-methylpiperazin-1-yl)-1,2,3,4-tetrahydronaphthalen-2-yl]methanesulfonamide,
N-[4-methyl-5-({[(2R)-5-methyl-8-(4-methylpiperazin-1-yl)-1,2,3,4-tetrahydronaphthalen-2-yl]amino}sulfonyl)-1,3-thiazol-2-yl]acetamide,
N-[(2R)-5-methyl-8-(4-methylpiperazin-1-yl)-1,2,3,4-tetrahydronaphthalen-2-yl]thiophene-2-sulfonamide,
N-[(2R)-5-methyl-8-(4-methylpiperazin-1-yl)-1,2,3,4-tetrahydronaphthalen-2-yl]-3-nitrobenzenesulfonamide,
2,5-dimethoxy-N-[(2R)-5-methyl-8-(4-methylpiperazin-1-yl)-1,2,3,4-tetrahydronaphthalen-2-yl]benzenesulfonamide,
5-bromo-2-methoxy-N-[(2R)-5-methyl-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]-4-(phenylsulfonyl)thiophene-2-sulfonamide,
N-[(2R)-5-methyl-8-(4-methylpiperazin-1-yl)-1,2,3,4-tetrahydronaphthalen-2-yl]-5-(phenylsulfonyl)thiophene-2-sulfonamide,
N-[(2R)-5-methyl-8-(4-methylpiperazin-1-yl)-1,2,3,4-tetrahydronaphthalen-2-yl]-2,1,3-benzothiadiazole-4-sulfonamide,
N-[(2R)-5-methyl-8-(4-methylpiperazin-1-yl)-1,2,3,4-tetrahydronaphthalen-2-yl]-2,1,3-benzoxadiazole-4-sulfonamide,
5-isoxazol-3-yl-N-[(2R)-5-methyl-8-(4-methylpiperazin-1-yl)-1,2,3,4-tetrahydronaphthalen-2-yl]thiophene-2-sulfonamide,
2-methyl-N-[(2R)-5-methyl-8-(4-methylpiperazin-1-yl)-1,2,3,4-tetrahydronaphthalen-2-yl]-5-nitrobenzenesulfonamide,
3-methyl-N-[(2R)-5-methyl-g-(4-methylpiperazin-1-yl)-1,2,3,4-tetrahydronaphthalen-2-yl]benzenesulfonamide,
2-methyl-N-[(2R)-5-methyl-8-(4-methylpiperazin-1-yl)-1,2,3,4-tetrahydronaphthalen-2-yl]-4-nitrobenzenesulfonamide,
3-chloro-4-fluoro-N-[(2R)-5-methyl-8-(4-methylpiperazin-1-yl)-1,2,3,4-tetrahydronaphthalen-2-yl]benzenesulfonamide,
2,4-difluoro-N-[(2R)-methyl-8-(4-methylpiperazin-1-yl)-1,2,3,4-tetrahydronaphthalen-2-yl]benzenesulfonamide,
5-fluoro-2-methyl-N-[(2R)-5-methyl-8-(4-methylpiperazin-1-yl)-1,2,3,4-tetrahydronaphthalen-2-yl]benzenesulfonamide,
1-[(1S,4R)-7,7-dimethyl-2-oxobicyclo[2.2.1]hept-1-yl]-N-[(2R)-5-methyl-g-(4-methylpiperazin-1-yl)-1,2,3,4-tetrahydronaphthalen-2-yl]methanesulfonamide,
2-fluoro-N-[(2R)-5-methyl-8-(4-methylpiperazin-1-yl)-1,2,3,4-tetrahydronaphthalen-2-yl]benzenesulfonamide,
3,4-dimethoxy-N-[(2R)-5-methyl-g-(4-methylpiperazin-1-yl)-1,2,3,4-tetrahydronaphthalen-2-yl]benzenesulfonamide,
5-chloro-N-[(2R)-5-methyl-8-(4-methylpiperazin-1-yl)-1,2,3,4-tetrahydronaphthalen-2-yl]thiophene-2-sulfonamide,
2-chloro-6-methyl-N-[(2R)-5-methyl-8-(4-methylpiperazin-1-yl)-1,2,3,4-tetrahydronaphthalen-2-yl]benzenesulfonamide,
5-bromo-N-[(2R)-5-methyl-8-(4-methylpiperazin-1-yl)-1,2,3,4-tetrahydronaphthalen-2-yl]thiophene-2-sulfonamide,
4-chloro-N-[(2R)-5-methyl-8-(4-methylpiperazin-1-yl)-1,2,3,4-tetrahydronaphthalen-2-yl]-3-nitrobenzenesulfonamide,
3-fluoro-N-[(2R)-5-methyl-8-(4-methylpiperazin-1-yl)-1,2,3,4-tetrahydronaphthalen-2-yl]benzenesulfonamide,
N-[2-chloro-4-({[(2R)-5-methyl-8-(4-methylpiperazin-1-yl)-1,2,3,4-tetrahydronaphthalen-2-yl]amino}sulfonyl)phenyl]acetamide,
2-methoxy-5-methyl-N-[(2R)-5-methyl-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]-2-nitrobenzenesulfonamide,
3,4,5-trimethoxy-N-[(2R)-5-methyl-8-(4-methylpiperazin-1-yl)-1,2,3,4-tetrahydronaphthalen-2-yl]benzenesulfonamide,
4-bromo-N-[(2R)-5-methyl-8-(4-methylpiperazin-1-yl)-1,2,3,4-tetrahydronaphthalen-2-yl]benzenesulfonamide,
2-bromo-N-[(2R)-5-methyl-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]-4-(trifluoromethyl)benzenesulfonamide,
4-ethyl-N-[(2R)-5-methyl-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]-2-nitro-4-(trifluoromethyl)benzenesulfonamide,
4-methyl-N-[(2R)-5-methyl-8-(4-methylpiperazin-1-yl)-1,2,3,4-tetrahydronaphthalen-2-yl]-3-nitrobenzenesulfonamide,
N-[(2R)-5-methyl-8-(4-methylpiperazin-1-yl)-1,2,3,4-tetrahydronaphthalen-2-yl]naphthalene-1-sulfonamide,
N-[(2R)-5-methyl-8-(4-methylpiperazin-1-yl)-1,2,3,4-tetrahydronaphthalen-2-yl]-2-oxo-1,2,3,4-tetrahydroquinoline-6-sulfonamide,
1-[(1R,4S)-7,7-dimethyl-2-oxobicyclo[2.2.1]hept-1-yl]-N-[(2R)-5-methyl-8-(4-methylpiperazin-1-yl)-1,2,3,4-tetrahydronaphthalen-2-yl]methanesulfonamide,
3,4-difluoro-N-[(2R)-5-methyl-8-(4-methylpiperazin-1-yl)-1,2,3,4-tetrahydronaphthalen-2-yl]benzenesulfonamide,
2-methyl-N-[(2R)-5-methyl-8-(4-methylpiperazin-1-yl)-1,2,3,4-tetrahydronaphthalen-2-yl]benzenesulfonamide,
2,5-dimethyl-N-[(2R)-5-methyl-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]-4-(pyridin-3-yloxy)benzenesulfonamide,
4-chloro-2,5-dimethoxy-N-[(2R)-5-methyl-8-(4-methylpiperazin-1-yl)-1,2,3,4-tetrahydronaphthalen-2-yl]benzenesulfonamide,
6-chloro-N-[(2R)-5-methyl-8-(4-methylpiperazin-1-yl)-1,2,3,4-tetrahydronaphthalen-2-yl]-2H-1,2,4-benzothiadiazine-7-sulfonamide 1,1-dioxide, methyl 2-methyl-5-({[(2R)-5-methyl-g-(4-methylpiperazin-1-yl)-1,2,3,4-tetrahydronaphthalen-2-yl]amino}sulfonyl)-3-furoate,
2-methoxy-5-({[(2R)-5-methyl-8-(4-methylpiperazin-1-yl)-1,2,3,4-tetrahydronaphthalen-2-yl]amino}sulfonyl)benzamide,
3-cyano-4-fluoro-N-[(2R)-5-methyl-8-(4-methylpiperazin-1-yl)-1,2,3,4-tetrahydronaphthalen-2-yl]benzenesulfonamide,
3-fluoro-4-methyl-N-[(2R)-5-methyl-8-(4-methylpiperazin-1-yl)-1,2,3,4-tetrahydronaphthalen-2-yl]benzenesulfonamide,
4-fluoro-2-methyl-N-[(2R)-5-methyl-8-(4-methylpiperazin-1-yl)-1,2,3,4-tetrahydronaphthalen-2-yl]benzenesulfonamide,
2-methoxy-N-[(2R)-5-methyl-8-(4-methylpiperazin-1-yl)-1,2,3,4-tetrahydronaphthalen-2-yl]-4-nitrobenzenesulfonamide,
2,4,5-trifluoro-N-[(2R)-5-methyl-8-(4-methylpiperazin-1-yl)-1,2,3,4-tetrahydronaphthalen-2-yl]benzenesulfonamide,
methyl 3-[4-({[(2R)-5-methyl-8-(4-methylpiperazin-1-yl)-1,2,3,4-tetrahydronaphthalen-2-yl]amino}sulfonyl)phenyl]propanoate,
N-[(2R)-5-methyl-8-(4-methylpiperazin-1-yl)-1,2,3,4-tetrahydronaphthalen-2-yl]-6-phenoxypyridine-3-sulfonamide,
N-[(2R)-5-methyl-8-(4-methylpiperazin-1-yl)-1,2,3,4-tetrahydronaphthalen-2-yl]-2,3-dihydro-1,4-benzodioxine-6-sulfonamide,
N-[(2R)-5-methyl-8-(4-methylpiperazin-1-yl)-1,2,3,4-tetrahydronaphthalen-2-yl]-1-benzofuran-2-sulfonamide,
4-chloro-N1-[(2R)-5-methyl-8-(4-methylpiperazin-1-yl)-1,2,3,4-tetrahydronaphthalen-2-yl]benzene-1,3-disulfonamide,
4-fluoro-N-[(2R)-5-methyl-8-(4-methylpiperazin-1-yl)-1,2,3,4-tetrahydronaphthalen-2-yl]naphthalene-1-sulfonamide,
3,5-difluoro-N-[(2R)-5-methyl-8-(4-methylpiperazin-1-yl)-1,2,3,4-tetrahydronaphthalen-2-yl]benzenesulfonamide,
4-fluoro-N-[(2R)-5-methyl-8-(4-methylpiperazin-1-yl)-1,2,3,4-tetrahydronaphthalen-2-yl]-3-(trifluoromethyl)benzenesulfonamide,
2-chloro-4,5-difluoro-N-[(2R)-5-methyl-8-(4-methylpiperazin-1-yl)-1,2,3,4-tetrahydronaphthalen-2-yl]benzenesulfonamide,
5-chloro-2,4-difluoro-N-[(2R)-5-methyl-8-(4-methylpiperazin-1-yl)-1,2,3,4-tetrahydronaphthalen-2-yl]benzenesulfonamide,
4-chloro-2,5-difluoro-N-[(2R)-5-methyl-8-(4-methylpiperazin-1-yl)-1,2,3,4-tetrahydronaphthalen-2-yl]benzenesulfonamide,
3-chloro-4-methyl-N-[(2R)-5-methyl-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]-2-(methylsulfonyl)benzenesulfonamide,
1,3,5-trimethyl-N-[(2R)-5-methyl-8-(4-methylpiperazin-1-yl)-1,2,3,4-tetrahydronaphthalen-2-yl]-1H-pyrazole-4-sulfonamide,
N-[(2R)-5-methyl-8-(4-methylpiperazin-1-yl)-1,2,3,4-tetrahydronaphthalen-2-yl]-1-(3-nitrophenyl)methanesulfonamide,
5-methyl-N-[(2R)-5-methyl-8-(4-methylpiperazin-1-yl)-1,2,3,4-tetrahydronaphthalen-2-yl]-2,1,3-benzothiadiazole-4-sulfonamide,
2,5-dimethoxy-N-[(2R)-5-methyl-8-(4-methylpiperazin-1-yl)-1,2,3,4-tetrahydronaphthalen-2-yl]-4-nitrobenzenesulfonamide,
N-[(2R)-5-methyl-8-(4-methylpiperazin-1-yl)-1,2,3,4-tetrahydronaphthalen-2-yl]-1-oxo-1,2-dihydroisoquinoline-4-sulfonamide,
dimethyl 5-({[(2R)-5-methyl-8-(4-methylpiperazin-1-yl)-1,2,3,4-tetrahydronaphthalen-2-yl]amino}sulfonyl)isophthalate,
4-methyl-N-[(2R)-5-methyl-8-(4-methylpiperazin-1-yl)-1,2,3,4-tetrahydronaphthalen-2-yl]benzenesulfonamide,
4-methoxy-N-[(2R)-5-methyl-8-(4-methylpiperazin-1-yl)-1,2,3,4-tetrahydronaphthalen-2-yl]benzenesulfonamide,
2-chloro-N-[(2R)-5-methyl-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]-3-(trifluoromethyl)benzenesulfonamide,
N-[(2R)-5-methyl-8-(4-methylpiperazin-1-yl)-1,2,3,4-tetrahydronaphthalen-2-yl]-5-pyridin-2-ylthiophene-2-sulfonamide,
2-cyano-N-[(2S)-5-methyl-8-(4-methylpiperazin-1-yl)-1,2,3,4-tetrahydronaphthalen-2-yl]benzenesulfonamide,
4-bromo-2-fluoro-N-[(2R)-5-methyl-8-(4-methylpiperazin-1-yl)-1,2,3,4-tetrahydronaphthalen-2-yl]benzenesulfonamide,
N-[3-({[(2R)-5-methyl-8-(4-methylpiperazin-1-yl)-1,2,3,4-tetrahydronaphthalen-2-yl]amino}sulfonyl)phenyl]acetamide,
3-chloro-2-methyl-N-[(2R)-5-methyl-8-(4-methylpiperazin-1-yl)-1,2,3,4-tetrahydronaphthalen-2-yl]benzenesulfonamide,
4-methoxy-N-[(2R)-5-methyl-8-(4-methylpiperazin-1-yl)-1,2,3,4-tetrahydronaphthalen-2-yl]-2-nitrobenzenesulfonamide,
3-methoxy-N-[(2R)-5-methyl-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]-2-(phenylsulfonyl)benzenesulfonamide,
(E)-N-[(2R)-5-methyl-8-(4-methylpiperazin-1-yl)-1,2,3,4-tetrahydronaphthalen-2-yl]-2-phenylethylenesulfonamide,
2-methoxy-4-methyl-N-[(2R)-5-methyl-8-(4-methylpiperazin-1-yl)-1,2,3,4-tetrahydronaphthalen-2-yl]benzenesulfonamide,
3-difluoromethoxy)-N-[(2R)-5-methyl-8-(4-methylpiperazin-1-yl)-1,2,3,4-tetrahydronaphthalen-2-yl]benzenesulfonamide,
4-bromo-3-fluoro-N-[(2R)-5-methyl-8-(4-methylpiperazin-1-yl)-1,2,3,4-tetrahydronaphthalen-2-yl]benzenesulfonamide,
N-[(2R)-5-methyl-8-(4-methylpiperazin-1-yl)-1,2,3,4-tetrahydronaphthalen-2-yl]-5-(1,2,3-thiadiazol-4-yl)thiophene-2-sulfonamide, and
methyl 2,5-dimethyl-4-({[(2S)-5-methyl-8-(4-methylpiperazin-1-yl)-1,2,3,4-tetrahydronaphthalen-2-yl]amino}sulfonyl)-3-furoate,
   or salts, solvates or solvated salts thereof.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

The term “Cₘ₋ₙ” or “Cₘ₋ₙ group” used alone or as a prefix, refers to any group having m to n carbon atoms.

The term “alkyl” used alone or as a suffix or prefix, refers to a saturated monovalent straight or branched chain hydrocarbon radical comprising from 1 to about 10 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl.

The term “C₀” means a bond or does not exist. For example “arylC₀alkyl” is equivalent with “aryl” and “C₂alkylOC₀alkyl” is equivalent with “C₂alkylO”.

As used herein, “alkenyl” refers to an unsaturated carbon chain having one or more double carbon-carbon bonds and includes both straight and branched chain alkenyl groups. Example on alkenyl groups includes, but is not limited to, vinyl, allyl, propenyl, butenyl, pentenyl, or hexenyl, and a butenyl group may for example be buten-2-yl, buten-3-yl or buten-4-yl.

As used herein, “alkynyl” refers to an unsaturated carbon chain having one or more triple carbon-carbon bonds and includes both straight and branched chain alkynyl groups. Example on alkynyl groups includes, but is not limited to, etynyl, propargyl, pentynyl or hexynyl and a butynyl group may for example be butyn-3-yl or butyn-4-yl.

As used herein, the term “alkoxy”, unless stated otherwise, refers to radicals of the general formula —O—R, wherein R is selected from a hydrocarbon radical. The term “alkoxy” may include, but is not limited to, methoxy, ethoxy, propoxy, isopropoxy, butoxy, t-butoxy, isobutoxy, cyclopropylmethoxy, allyloxy or propargyloxy.

In this specification, unless stated otherwise, the term “amine” or “amino” refers to radicals of the general formula —NRR′, wherein R and R′ are selected independently from hydrogen or a hydrocarbon radical.

In this specification, unless stated otherwise, the term “cycloalkyl” refers to an optionally substituted, partially or completely saturated monocyclic, bicyclic or bridged hydrocarbon ring system. The term “C₃₋₈cycloalkyl” may be, but is not limited to, cyclopropyl, cyclobutyl, cyclopentyl, cyclohexyl, cycloheptyl, cyclopentenyl or norbornyl.

In this specification, unless stated otherwise, the term “heterocycloalkyl” refers to an optionally substituted, partially or completely saturated monocyclic, bicyclic or bridged hydrocarbon ring system containing at least one heteroatom selected independently from N, O or S.

In this specification, unless stated otherwise, the term “aryl” refers to an optionally substituted monocyclic or bicyclic hydrocarbon ring system with at least one unsaturated aromatic ring. Examples of “aryl” may be, but are not limited to phenyl, naphthyl or tetralinyl.

In this specification, unless stated otherwise, the term “heteroaryl” refers to an optionally substituted monocyclic or bicyclic hydrocarbon ring system with at least one unsaturated aromatic ring and containing at least one heteroatom selected independently from N, O or S. Examples of “heteroaryl” may be, but are not limited to pyridyl, pyrrolyl, furyl, thienyl, imidazolyl, oxazolyl, isoxazolyl, thiazolyl, pyrazolyl, benzofuryl, indolyl, isoindolyl, benzimidazolyl, pyridazinyl, pyrimidinyl, pyrazinyl, tetrazolyl, triazolyl, quinazolinyl or isothiazolyl. For the avoidance of doubt, a C₅heteroaryl refers to a 5 membered aromatic ring system containing at least one heteroatom.

In this specification, unless stated otherwise, the terms “arylalkyl” and “heteroarylalkyl” refer to a substituent that is attached via the alkyl group to an aryl or heteroaryl group.

In this specification, unless stated otherwise, the terms “halo” and “halogen” may be fluoro, iodo, chloro or bromo.

In this specification, unless stated otherwise, the term “haloalkyl” means an alkyl group as defined above, which is substituted with halogen as defined above. The term “C₁₋₆haloalkyl” may include, but is not limited to fluoromethyl, difluoromethyl, trifluoromethyl, fluoroethyl, difluoroethyl or bromopropyl. The term “C₁₋₆haloalkylO” may include, but is not limited to fluoromethoxy, difluoromethoxy, trifluoromethoxy, fluoroethoxy or difluoroethoxy.

The present invention relates to the compounds of formula I as hereinbefore defined as well as to the salts, solvates or solvated salts thereof. Salts for use in pharmaceutical formulations will be pharmaceutically acceptable salts, but other salts may be useful in the production of the compounds of formula I.

A suitable pharmaceutically acceptable salt of the compounds of the invention is, for example, an acid-addition salt, for example a salt with an inorganic or organic acid. In addition, a suitable pharmaceutically acceptable salt of the compounds of the invention is an alkali metal salt, an alkaline earth metal salt or a salt with an organic base. Other pharmaceutically acceptable salts and methods of preparing these salts may be found in, for example, Remington's Pharmaceutical Sciences (18^{th} Edition, Mack Publishing Co.).

Some compounds of formula I may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of formula I.

### Methods of Preparation

### General Procedure

Throughout the following description of such processes it is to be understood that, where appropriate, suitable protecting groups will be added to, and subsequently removed from, the various reactants and intermediates in a manner that will be readily understood by one skilled in the art of organic synthesis. The specific sequence of reactions depicted under “General procedure” is not critical. For many of the compounds described the order of the reaction steps may be varied. The reactions are run until judged complete by LC-UV, LC-MS, TLC or NMR.

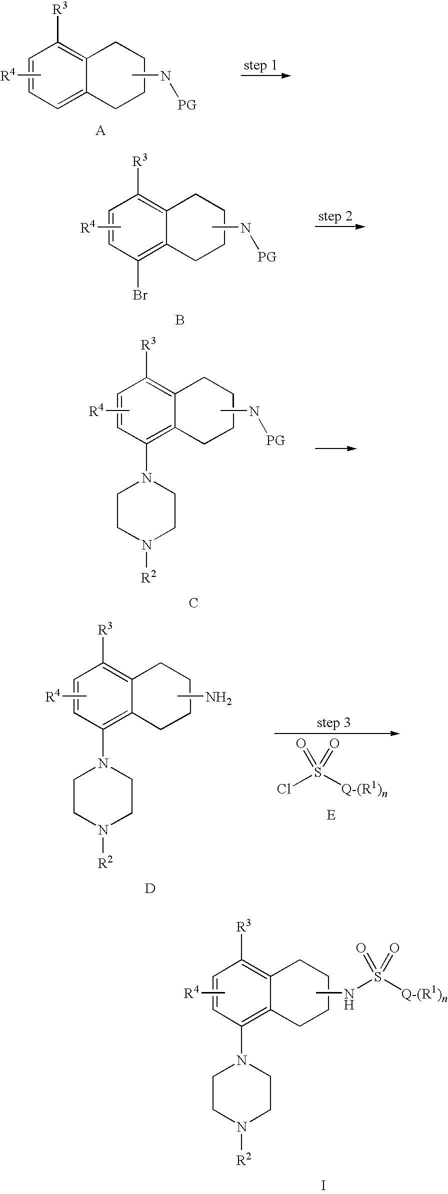

### Step 1

A compound A, wherein R³ and R⁴ are as defined above, and wherein PG is a protection group, such as, but not limited to, tert-butoxycarbonyl, benzyloxycarbonyl, 2,2,2-trichloromethylcarbonyl, 2,2,2-trifluoromethylcarbonyl, allyl, benzyl, phenethyl, p-methoxybenzyl, p-nitrobenzyl, diphenylmethyl, may be transformed into a compound B, wherein R³ and R⁴ are as defined above, by bromination using bromine in a solvent such as acetic acid, optionally in the presence of sodium acetate. Other solvents that may be used are for example water, dichloromethane or dioxane. The reaction may be performed at temperatures between 0° C. and the reflux temperature of the solvent. The product may be isolated by precipitation, extraction or column chromatography.

### Step 2

A compound B, wherein R³ and R⁴ are as defined above and wherein PG is a protection group as defined above, may be transformed into a compound C, wherein R², R³ and R⁴ are as defined above and wherein PG is a protection group (for examples on suitable PG see step 1) as defined above, by the reaction with an appropriate piperazine. The reaction may be performed without solvent (neat), or in a solvent, such as toluene, THF, dioxane, 1,2-dimethoxyethane, o-xylene, or mixtures thereof, in the presence of a catalyst, such as Pd(OAc)₂, Pd₂(dba)₃, a ligand such as BINAP or bis(2-diphenylphosphinophenyl)ether, 1,3-bis(diphenylphosphino)propane, tri-t-butylphosphine, and a base, such as sodium t-butoxide, caesium carbonate, potassium carbonate, potassium phosphate or lithium bis(trimethylsilyl)amide. The reaction may be run at temperatures between 40° C. and the reflux temperature of the solvent, preferably under inert atmosphere. The reaction may also be accelerated by the use of microwave irradiation. The product may be isolated by extraction, precipitation or column chromatography.

### Step 3

A compound D, wherein R², R³ and R⁴ are as defined above, may be transformed into a compound I by reaction with a compound E, wherein R¹ and Q are as defined above, in a solvent, such as DMF, N-methylpyrrolidinone, acetonitrile, dioxane, chloroform or dichloromethane, or mixtures thereof, in the presence of a base, such as pyridine, triethylamine, PS-DIEA or DIPEA, at temperatures between 0° C. and the reflux temperature of the solvent. The product may be isolated by precipitation, extraction or column chromatography.

### Intermediates

A further embodiment of the invention relates to compounds selected from the group consisting of
(2R)—N,N-dibenzyl-5-bromo-8-methoxy-1,2,3,4-tetrahydronaphthalen-2-amine,
(2R)—N,N-dibenzyl-8-methoxy-5-(4-methylpiperazin-1-yl)-1,2,3,4-tetrahydronaphthalen-2-amine,
(2R)-8-methoxy-5-(4-methylpiperazin-1-yl)-1,2,3,4-tetrahydronaphthalen-2-amine,
N-[(2S)-5-bromo-8-methoxy-1,2,3,4-tetrahydronaphthalen-2-yl]-2,2,2-trifluoroacetamide, tert-butyl 4-[(6S)-4-methoxy-6-[(trifluoroacetyl)amino]-5,6,7,8-tetrahydronaphthalen-1-yl]piperazine-1-carboxylate, and
tert-butyl 4-[(6S)-6-amino-4-methoxy-5,6,7,8-tetrahydronaphthalen-1-yl]piperazine-1-carboxylate,
   which may be used as intermediates in the preparation of compounds suited for the is treatment of 5-HT6 mediated disorders, especially for use as intermediates for the preparation of compounds of formula I.

### Pharmaceutical Composition

According to one embodiment of the present invention there is provided a pharmaceutical composition comprising as active ingredient a therapeutically effective amount of the compound of formula I, or salts, solvates or solvated salts thereof, in association with one or more pharmaceutically acceptable diluents, excipients and/or inert carriers.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream, for rectal administration, e.g. as a suppository, or for inhalation.

In general, the above compositions may be prepared in a conventional manner using one or more conventional excipients, pharmaceutical acceptable diluents and/or inert carriers. Suitable daily doses of the compounds of formula I in the treatment of a mammal, including man, are approximately 0.01 to 250 mg/kg bodyweight at peroral administration and about 0.001 to 250 mg/kg bodyweight at parenteral administration.

The typical daily dose of the active ingredient varies within a wide range and will depend on various factors such as the relevant indication, severity of the illness being treated, the route of administration, the age, weight and sex of the patient and the particular compound being used, and may be determined by a physician.

### Medical Use

Interestingly, it has been found that the compounds according to the present invention are useful in therapy. The compounds of the present invention as defined hereinabove, or pharmaceutically acceptable salts, solvates or solvated salts thereof, as well as their corresponding active metabolites, exhibit a high degree of potency and selectivity for 5-hydroxy-tryptamine 6 (5-HT6) receptors. Accordingly, the compounds of the present invention are expected to be useful in the treatment of conditions associated with excessive activation of 5-HT6 receptors.

The compounds the present invention may be used to produce an inhibitory effect of 5-HT6 receptors in mammals, including man.

The compounds of the present invention as defined hereinabove are expected to be suitable for the treatment of disorders relating to or affected by the 5-HT6 receptor including cognitive, personality, behaviour, psychiatric and neurodegenerative disorders.

Examples of such disorder may be selected from the group comprising of Alzheimer's disease anxiety, depression, convulsive disorders such as epilepsy, personality disorders, obsessive compulsive disorders, migraine, cognitive disorders such as memory dysfunction, sleep disorders, feeding disorders such as anorexia, obesity, bulimia, panic attacks, withdrawal from drug abuse, schizophrenia, attention deficit hyperactive disorder (ADUD), attention deficit disorder (ADD), dementia, memory loss, disorders associated with spinal trauma and/or head injury, stroke, diabetes type 2, binge disorders, bipolar disorders, psychoses, Parkinson's disease, Huntington's disease, neurodegenerative disorders characterized by impaired neuronal growth, and pain.

Further relevant disorders may be selected from the group comprising gastrointestinal disorders such as gastro-esophageal reflux disease (GERD) and irritable bowel syndrome (IBS).

A compound the present invention as defined hereinabove may also be used for treatment of tolerance to 5-HT6 activators.

One embodiment of the invention relates to a compound of the present invention as hereinbefore defined, for use in therapy.

Another embodiment of the invention relates to a compound of the present invention as hereinbefore defined, for use in treatment of 5-HT6 mediated disorders.

A further embodiment of the invention relates to a compound of the present invention as hereinbefore defined, for use in treatment of Alzheimer's disease.

Another embodiment of the invention relates to the compounds of the present invention as hereinbefore defined, for use in treatment of cognitive impairment associated with schizophrenia.

Yet a further embodiment of the invention relates to a compound of the present invention as hereinbefore defined, for use in treatment of obesity.

One embodiment of the invention relates to a compound of the present invention as hereinbefore defined, for use in Parkinson's disease.

Another embodiment of the invention relates to the use of a compound of the present invention as hereinbefore defined, in the manufacture of a medicament for treatment of 5-HT6 mediated disorders, Alzheimer's disease, cognitive impairment associated with schizophrenia, obesity and/or Parkinson's disease, and any other disorder mentioned above.

A further embodiment of the invention relates to a method of treatment of 5-HT6 mediated disorders, Alzheimer's disease, cognitive impairment associated with schizophrenia, obesity and/or Parkinson's disease, and any other disorder mentioned above, comprising administering to a mammal, including man in need of such treatment, a therapeutically effective amount of a compound of the present invention, as hereinbefore defined.

Yet another embodiment of the invention relates to a pharmaceutical composition comprising a compound of the present invention as hereinbefore defined, for use in treatment of 5-HT6 mediated disorders, Alzheimer's disease, cognitive impairment associated with schizophrenia, obesity and/or Parkinson's disease, and any other disorder mentioned above.

One embodiment of the invention relates to an agent for the prevention or treatment of 5-HT6 mediated disorders, Alzheimer's disease, cognitive impairment associated with schizophrenia, obesity and/or Parkinson's disease, and any other disorder mentioned above, which comprises as active ingredient a compound of the present invention as hereinbefore defined.

In the context of the present specification, the term “therapy” and “treatment” includes prevention and prophylaxis, unless there are specific indications to the contrary. The terms “treat”, “therapeutic” and “therapeutically” should be construed accordingly.

In this specification, unless stated otherwise, the terms “inhibitor” and “antagonist” mean a compound that by any means, partly or completely, blocks the transduction pathway leading to the production of a response by the agonist.

The compounds according to the present invention are modulators of the 5-HT6 receptors, and may be inhibitors, as well as agonists, inverse-agonists or partial-agonist.

The term “disorder”, unless stated otherwise, means any condition and disease associated with 5-HT6 receptor activity.

### Non-Medical Use

In addition to their use in therapeutic medicine, the compounds of formula I, or salts, solvates or solvated salts thereof, are also useful as pharmacological tools in the development and standardisation of in vitro and in vivo test systems for the evaluation of the effects of modulators of 5-HT6 related activity in laboratory animals such as cats, dogs, rabbits, monkeys, rats and mice, as part of the search for new therapeutics agents.

### EXAMPLES

### General Methods

The invention will now be illustrated by the following non-limiting Examples in which, generally
(i) operations were carried out at ambient or room temperature, i.e. in the range 17 to 25° C. and under an atmosphere of an inert gas such as argon unless otherwise stated;
(ii) evaporations were carried out by rotary evaporation in vacuo and work-up procedures were carried out after removal of residual solids by filtration;
(iii) HPLC analyses were performed on an Agilent HP 1000 system consisting of G1379A Micro Vacuum Degasser, G1312A Binary Pump, G1367A Wellplate auto-sampler, G316A Thermostatted Column Compartment and G1315B Diode Array Detector. Column: X-Terra MS, Waters, 4.6×50 mm, 3.5 μm. The column temperature was set to 40° C. and the flow rate to 1.5 ml/min. The Diode Array Detector was scanned from 210-300 nm, step and peak width were set to 2 nm and 0.05 min, respectively. A linear gradient was applied, run from 0% to 100% acetonitrile, in 4 min. Mobile phase: acetonitrile/10 mM ammonium acetate in 5% acetonitrile in MilliQ Water.
(iv) Thin layer chromatography (TLC) was performed on Merck TLC-plates (Silica gel 60 F₂₅₄) and UV visualized the spots. Flash chromatography was preformed on a Combi Flash® Companion™ using RediSep™ normal-phase flash columns or on Merck Silica gel 60 (0.040-0.063 mm). Typical solvents used for flash chromatography were mixtures of chloroform/methanol, toluene/ethyl acetate and ethyl acetate/hexanes.
(v) ¹H and ¹³C NMR spectra were recorded at 400 MHz for proton and 100 MHz for carbon-13 either on a Varian Unity+400 NMR Spectrometer equipped with a 5 mm BBO probe with Z-gradients, or a Bruker Avance 400 NMR spectrometer equipped with a 60 μl dual inverse flow probe with Z-gradients, or a Bruker DPX400 NMR spectrometer equipped with a 4-nucleus probe equipped with Z-gradients. Unless specifically noted in the examples, spectra were recorded at 400 MHz for proton and 100 MHz for carbon-13. The following reference signals were used: the middle line of DMSO-d₆ δ 2.50 (¹H); the middle line of CD₃OD δ 3.31 (¹H); acetone-d₆ 2.04 (¹H); and CDCl₃ δ 7.26 (1H) (unless otherwise indicated);
(vi) Mass spectra were recorded on a Waters LCMS consisting of an Alliance 2795 (LC) and a ZQ single quadrupole mass spectrometer. The mass spectrometer was equipped with an electrospray ion source (ESI) operated in a positive or negative ion mode. The capillary voltage was 3 kV and the mass spectrometer was scanned from m/z 100-700 with a scan time of 0.3 or 0.8 s. Separations were performed on either Waters X-Terra MS, C8-columns, (3.5 μm, 50 or 100 mm×2.1 mm i.d.), or a ScantecLab's ACE3AQ column (100 mm×2.1 mm i.d.). The column temperature was set to 40° C. A linear gradient was applied using a neutral or acidic mobile phase system, running at 0% to 100% organic phase in 4-5 minutes, flow rate 0.3 ml/min. Mobile phase system: acetonitrile/[10 mM NH₄OAc (aq.)/MeCN (95:5)], or [10 mM NH₄OAc (aq.)/MeCN (1/9)]/[10 mM OAc(aq.)/MeCN(9/11)]. Acidic mobile phase system: [133 mMHCOOH(aq.)/MeCN(5/95)]/[8 mMHCOOH(aq.)/IMeCN(98/2)];
(vii) Alternatively a LC-MS system (Sample Manager 2777C, 152511 binary pump, 1500 Column Oven, ZQ, PDA2996 and ELS detector, Sedex 85) from Waters was used. Separation was performed using a Zorbax column (C8, 3.0×50 nm u, 3 μm). A four minutes linear gradient was used starting at 100% A (A=10 mM NH₄OAc in 5% MeOH) and ending at 100% B (MeOH). The ZQ was equipped with a combined APPI/APCI ion source and scanned in the positive mode between m/z 120-800 with a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode;
(viii) Preparative chromatography was run on a Gilson auto-preparative HPLC with a diode array detector. Column: XTerra MS C8, 19×300 mm, 7 μm. Gradient with acetonitrile/0.1M ammonium acetate in 5% acetonitrile in MilliQ Water, run from 20% to 60% acetonitrile, in 13 min. Flow rate: 20 ml/min. Alternatively, purification was achieved on a semi preparative Shirnadzu LC-8A HPLC with a Shimadzu SPD-10A TV-vis.-detector equipped with a Waters Symmetry® column (C18, 5 μm, 100 mm×19 mm). Gradient with acetonitrile/0.1% trifluoroacetic acid in MilliQ Water, run from 35% to 60% acetonitrile in 20 min. Flow rate: 10 ml/min;
(ix) For the compounds in example 36-151 the following equipment was used: The structure and purity of all intermediates were assessed by HPLC and NM analysis. ¹H NMR spectra were determined using a 300 MHz and/or 400 MHz Varian Unity Inova spectrometer with 4-nucleus 5 mm probes installed. LC/MS were performed on Agilent 1100 series HPLC equipped with a 4.6×50 3.5 micron XTerra® MS C8 analytical reverse-phase column (Waters), using a gradient of acetonitrile and a solution of 0.2% 880 ammonia in water at 2 ml/min. Agilent MSD APCI was used for MS detection; both positive and negative ion data were collected when appropriate. All purities of the final products were analysed using a Agilent 1100 series high throughout system, containing: Agilent 1100 series well plate handler, Agilent 1100 series autointerface, Agilent 1100 series well plate autosampler, 2× Agilent 1100 series binary pumps, Agilent 1100 series thermostatted column compartment, Agilent 1100 series diode array detector, Agilent 1100 series mass spectrometer. The stationary phase used was 4.6×20 mm XTerra® MS C₈ IS columns (Waters) analytical reversed-phase column and the mobile phase used was 0.1% 880 ammonia and acetonitrile with UV detection at 220 nm, MS detection with APCI ionisation in positive scan mode. The structures of the final products were confirmed by ¹H NMR spectroscopy recorded using Varian Unity Inova 500 MHz spectrometer, equipped with a 60 ul triple resonance flow probe ant the samples were transferred to the flow cell by direct injection with a Gilson 215 liquids handler. Samples were prepared in 20 ul h₆-DMSO+170 ul d₆-DMSO to a final concentration of 2.6 mM. h₆-DMSO is used for the push solvent. Proton NMR spectra were acquired with WET solvent suppression on both the DMSO and H₂O signals, using Scout-Scan to find the solvent resonances. Spectra were acquired at 25° C.;
(x) All solvents used were analytical grade and commercially available anhydrous solvents for reactions. Reactions were typically run under an inert atmosphere of nitrogen or argon; yields, where present, are not necessarily the maximum attainable;
(xii) intermediates were not necessarily fully purified but their structures and purity were assessed by thin layer chromatographic, HPLC, infra-red (IR), MS and/or NMR analysis;
(xiii) melting points are uncorrected and were determined using a Mettler SP62 automatic melting point apparatus or an oil-bath apparatus; melting points for the end-products of the Formula I were determined after crystallisation from an appropriate organic solvent or solvent mixture;
(xiv) the following abbreviations have been used:
   HPLC high performance liquid chromatography
   LC liquid chromatography
   MS mass spectrometry
   ret. time retention time
   TFA trifluroacetic acid
   TEA triethylamine
   THF tetrahydrofurane
   DMF dimethylformamide
   DIPEA N,N-diisopropylethylamine
   DMSO dimethylsulfoxide
   NMP 1-methyl-2-pyrrolidinone
   THF tetrahydrofuran
   MeOH methanol
   RT room temperature
   Na₂SO₄ sodium sulfate
   EtOAc Ethyl acetate
   H₂O water

Throughout the following description of such processes it is to be understood that, where appropriate, suitable protecting groups, such as, but not limited to, tert-butoxycarbonyl, benzyloxycarbonyl, 2,2,2-trichloromethylcarbonyl, 2,2,2-trifluoromethylcarbonyl, allyl, benzyl, phenethyl, p-methoxybenzyl, p-nitrobenzyl, diphenylmethyl will be added to, and subsequently removed from, the various reactants and intermediates in a manner that will be readily understood by one skilled in the art of organic synthesis. The specific sequence of reactions depicted is not critical. For many of the compounds described the order of the reaction steps may be varied.

### Starting Materials

(2R)-8-methoxy-1,2,3,4-tetrahydronaphthalen-2-amine hydrochloride (WO9734883); (2S)-5-(4-methylpiperazin-1-yl)-1,2,3,4-tetrahydronaphthalen-2-amine (WO9734883); (2R)-5-methyl-8-(4-methylpiperazin-1-yl)-1,2,3,4-tetrahydronaphthalen-2-amine (WO9905134); tert-butyl 4-[(7R)-7-amino-4-methyl-5,6,7,8-tetrahydronaphthalen-1-yl]piperazine-1-carboxylate (WO9905134); tert-Butyl 4-[(7R)-7-amino-5,6,7,8-tetrahydronaphthalen-1-yl]piperazine-1-carboxylate (WO9734883)

Other starting materials used were either available from commercial sources or prepared according to literature procedures.

### Example 1

### 3-Chloro-N-[(2R)-8-methoxy-5-(4-methylpiperazin-1-yl)-1,2,3,4-tetrahydronaphthalen-2-yl]benzenesulfonamide

(2R)-8-Methoxy-5-(4-methylpiperazin-1-yl)-1,2,3,4-tetrahydronaphthalen-2-amine (41 mg, 0.15 mmol) and TEA (23 μl, 0.16 mmol) were dissolved in dioxane (1 ml) and 3-chlorobenzenesulfonyl chloride (22 μl, 0.16 mmol) was added. The mixture was stirred at ambient temperature for 20 h. Methanol was added and the mixture was purified by preparative HPLC to give the acetate of the title compound as a dry film (53 mg, 79%).

¹H NMR (400 MHz, CDCl₃) δ ppm 7.90 (1H; t) 7.80 (1H, d) 7.56 (1H, d) 7.44-7.49 (I H, m) 6.95 (1H, d) 6.64 (1H, d) 4.86 (1H, br. s.) 3.73 (3H, s) 3.64 (1H, br. s.) 2.75-3.03 (10H, m) 2.60-2.70 (1H, m) 2.51 (3H, s) 2.40-2.49 (1H, m) 1.92-2.01 (1H, m) 1.67-1.78 (1H, m); ESI-MS m/z M+H⁺ 450, 452.

### Example 2

### 4-Chloro-N-[(2R)-8-methoxy-5-(4-methylpiperazin-1-yl)-1,2,3,4-tetrahydronaphthalen-2-yl]benzenesulfonamide

The title compound was prepared according to the method described in Example 1 and a dry film (50 mg, 75%) was obtained.

¹H NMR (600 MHz, MeOD-d₄) δ ppm 7.82-7.85 (2H, m) 7.49-7.52 (2H, m) 6.89 (1H, d) 6.63 (1H, d) 3.70 (3H, s) 3.41-3.47 (1H, m) 2.58-2.99 (10H, m) 2.50-2.57 (1H, m) 2.43 (3H, s) 2.33-2.39 (1H, m) 1.87-1.92 (1H, m) 1.54-1.62 (1H, m); ESI-MS m/z M+H⁺ 450, 452.

### Example 3

### 3-Chloro-N-[(2S)-5-(4-methylpiperazin-1-yl)-1,2,3,4-tetrahydronaphthalen-2-yl]benzenesulfonamide

(2S)-5-(4-Methylpiperazin-1-yl)-1,2,3,4-tetrahydronaphthalen-2-amine (24 mg, 0.10 mmol), 3-chlorobenzenesulfonyl chloride (16 μl, 0.11 mmol) and TEA (16 μl, 0.11 mmol) were dissolved in DMF (0.7 ml). The mixture was stirred at ambient temperature for 20 h. Methanol (0.2 ml) was added and the mixture was purified by preparative HPLC. The fractions containing product were pooled, the acetonitrile was removed by evaporation and the remaining aqueous solution was extracted with dichloromethane (3 times). The organic phase was dried (using Na₂SO₄) and the solvent was evaporated to give a dry film (16 mg, 38%).

¹H NMR (400 MHz, MeOD-d₄) δ ppm 7.90 (1H, t) 7.83 (1H, d) 7.62-7.67 (1H, m) 7.54-7.59 (1H, m) 7.05 (1H, t) 6.90 (1H, d) 6.71 (1H, d) 3.50-3.58 (1H, m) 2.81-3.00 (7H, m) 2.55-2.78 (5H, m) 2.44 (3H, s) 1.84-1.92 (1H, m) 1.56-1.67 (1H, m), ESI-MS m/z M+H⁺ 420, 422.

### Examples 4

### 3-Chloro-4-methyl-N-[(2S)-5-(4-methylpiperazin-1-yl)-1,2,3,4-tetrahydronaphthalen-2-yl]benzenesulfonamide

The title compound was prepared according to the method described in Example 3. The yield (%) was 62.

¹H NMR (400 MHz, CDCl₃) δ ppm 7.88 (1H, s) 7.64-7.71 (1H, m) 7.33-7.41 (1H, m) 7.05-7.13 (1H, m) 6.88-6.94 (1H, m) 6.69-6.76 (1H, m) 4.86 (1H, br. s.) 3.64 (1H, br. s.) 2.83-3.02 (6H, m) 2.64 (6H, br. s.) 2.46 (3H, s) 2.37-2.42 (3H, m) 1.98-2.04 (1H, m) 1.66-1.79 (1H, m) ESI-MS m/z M+H⁺ 434, 436.

### Example 5

### 5-Chloro-N-[(2S)-5-(4-methylpiperazin-1-yl)-1,2,3,4-tetrahydronaphthalen-2-yl]naphthalene-2-sulfonamide

The title compound was prepared according to the method described in Example 3. The is yield (%) was 57.

¹H NMR (400 MHz, CDCl₃) δ ppm 8.49 (1H, d) 8.42 (1H, d) 7.99 (1H, dd) 7.90 (1H, d) 7.75 (1H, d) 7.54 (1H, t) 7.06 (1H, t) 6.90 (1H, d) 6.70 (1H, d) 5.16 (1H, br. s.) 3.69 (1H, br. s.) 2.54-3.03 (12H, m) 2.48 (3H, s) 1.91-2.00 (1H, m) 1.69-1.80 (1H, m) ESI-MS m/z M+H⁺ 470, 472.

### Example 6

### 3-Chloro-4-methyl-N-[(2S)-5-piperazin-1-yl-1,2,3,4-tetrahydronaphthalen-2-yl]benzenesulfonamide

3-Chloro-4-methyl-N-[(2S)-5-(4-methylpiperazin-1-yl)-1,2,3,4-tetrahydronaphthalen-2-yl]benzenesulfonamide (19 mg, 0.043 mmol) was dissolved in dichloroethane (0.6 ml) and chloroethyl chloroformate (15 μl, 0.13 mmol) was added. The mixture was heated at 80° C. for 1 h using microwave irradiation. Chloroethyl chloroformate (150 μl, 1.3 mmol) was added and the mixture was heated at 130° C. for 1 h using microwave irradiation. Methanol (1 ml) was added and the mixture was stirred at ambient temperature for 20 h. The solvent was evaporated and the residue was purified by preparative HPLC to give a dry film (6.5 mg, 31%).

¹H NMR (400 MHz, MeOD-d₄) δ ppm 7.87 (1H, d) 7.72 (1H, dd) 7.50 (1H, d) 7.09 (1H, t) 6.94 (1H, d) 6.77 (1H, d) 3.46-3.55 (1H, m) 3.24-3.36 (4H, m) 2.86-3.14 (6H, m) 2.58-2.69 (2H, m) 2.47 (3H, s) 1.87-1.92 (1H, m) 1.58-1.69 (1H, m); ESI-MS m/z M+H⁺ 420, 422.

### Example 7

### 3-Chloro-N-[(25)-5-piperazin-1-yl-1,2,3,4-tetrahydronaphthalen-2-yl]benzenesulfonamide

3-Chloro-N-[(2S)-5-(4-methylpiperazin-1-yl)-1,2,3,4-tetrahydronaphthalen-2-yl]benzenesulfonamide (215 mg, 0.512 mmol) was dissolved in chloroethyl chloroformate (1.66 ml, 15 mmol) and the mixture was heated at 130° C. for 2 h using microwave irradiation. Methanol (3 ml) was added and the mixture was stirred at ambient temperature over night. The solvent was evaporated and the residue was purified by preparative HPLC to give a solid (38 mg, 18%).

¹H NMR (400 MHz, CDCl₃) δ ppm 7.90 (1H, t) 7.80 (1H, d) 7.56 (1H, d) 7.44-7.50 (1H, m) 7.10 (1H, t) 6.90 (1H, d) 6.77 (1H, d) 3.60-3.67 (1H, m) 3.20 (4H, br. s.) 2.85-3.08 (7H, m) 2.56-2.72 (2H, m) 1.92-2.00 (1H, m) 1.66-1.77 (1H, m); ESI-MS m/z M+H⁺ 406, 408.

### Example 8

### 3-Chloro-N-[(2S)-8-methoxy-5-piperazin-1-yl-1,2,3,4-tetrahydronaphthalen-2-yl]benzenesulfonamide

### Example 8A

### tert-butyl 4-[(6S)-6-{[(3-chlorophenyl)sulfonyl]amino}-4-methoxy-5,6,7,8-tetrahydronaphthalen-1-yl]piperazine-1-carboxylate

tert-Butyl 4-[(6S)-6-amino-4-methoxy-5,6,7,8-tetrahydronaphthalen-1-yl]piperazine-1-carboxylate (150 mg, 0.41 mmol) and 3-chlorobenzenesulfonyl chloride (122 mg, 0.58 mmol) were dissolved in dichloromethane (8 ml). DIPEA (0.5 ml) was added and the mixture was stirred at ambient temperature for 10 h. Saturated aqueous sodium hydrogen carbonate was added and the phases were separated. The organic phase was dried (using Na₂SO₄) filtered and the solvent was evaporated. The residue was purified by column chromatography on silica eluting with gradients of EtOAc and heptane to give the intermediate tert-butyl 4-((6S)-6-{[(3-chlorophenyl)sulfonyl]amino}-4-methoxy-5,6,7,8-tetrahydronaphthalen-1-yl)piperazine-1-carboxylate ESI-MS m/z M+H⁺ 536, 538.

### Example 8B

### 3-Chloro-N-[(2S)-8-methoxy-5-piperazin-I-yl-1,2,3,4-tetrahydronaphthalen-2-yl]benzenesulfonamide

tert-Butyl 4-((6S)-6-{[(3-chlorophenyl)sulfonyl]amino}-4-methoxy-5,6,7,8-tetrahydronaphthalen-1-yl)piperazine-1-carboxylate, obtained from Example 8A was dissolved in dichloromethane (10 ml) and TFA (1 ml) was added. The mixture was stirred at ambient temperature for 6 h. The solvent was evaporated and the residue was dissolved in methanol. The solution was loaded on a SCX column and the column was eluted with 0.7 M ammonia in methanol. The solvent was evaporated and the residue was purified by column chromatography on silica eluting with gradients of 3% ammonia in methanol and chloroform to give a solid (54 mg, 30%).

¹H NMR (400 MHz, MeOD-d₄) δ ppm 7.88-7.91 (1H, m) 7.81-7.85 (1H, m) 7.62-7.66 (1H, m) 7.53-7.59 (1H, m) 6.89 (1H, d) 6.67 (1H, d) 3.71 (3H, s) 3.46-3.54 (1H, m) 2.88-3.00 (5H, m) 2.77-2.86 (3H, m) 2.71 (2H, br. s.) 2.53-2.63 (1H, m) 2.32-2.41 (1H, m) 1.81-1.89 (1H, m) 1.52-1.63 (1H, m); ESI-MS m/z M+H⁺ 436, 438.

### Examples 9

### N-[(2S)-8-methoxy-5-piperazin-1-yl-1,2,3,4-tetrahydronaphthalen-2-yl]-3,5-dimethylisoxazole-4-sulfonamide

The title compound was prepared according to the method described in Example 8. The yield was 30%.

¹H NMR (400 MHz, CDCl₃) δ ppm 6.95 (1H, d) 6.67 (1H, d) 4.63 (1H, br. s.) 3.76 (3H, s) 3.66 (1H, br. s.) 2.89-3.05 (6H, m) 2.68-2.86 (5H, m) 2.66 (3H, s) 2.49-2.58 (1H, m) 2.38 (3H, s) 1.97-2.07 (1H, m) 1.71-1.82 (1H, m) ESI-MS m/z M+H⁺ 421

### Example 10

### 1-[3-chloro-5-(trifluoromethyl)pyridin-2-yl]-N-[(2S)-8-methoxy-5-piperazin-1-yl-1,2,3,4-tetrahydronaphthalen-2-yl]-1H-pyrrole-2-sulfonamide

The title compound was prepared according to the method described in Example 8. The yield was 30%.

¹H NMR (400 MHz, CDCl₃) δ ppm 8.70 (1H, s) 8.15 (1H, d) 8.07 (1H, t) 7.55-7.58 (1H, m) 6.92 (1H, d) 6.64-6.68 (2H, m) 4.48 (1H, d) 3.75 (3H, s) 3.68-3.74 (1H, m) 2.94-3.06 (6H, m) 2.66-2.85 (5H, m) 2.48-2.56 (1H, m) 2.01-2.10 (1H, m) 1.72-1.82 (1H, m) ESI-MS m/z M+H⁺ 570, 572.

### Example 11

### 2,3-Dichloro-N-[(2R)-5-methyl-8-(4-methylpiperazin-1-yl)-1,2,3,4-tetrahydronaphthalen-2-yl]benzenesulfonamide

(2R)-5-Methyl-8-(4-methylpiperazin-1-yl)-1,2,3,4-tetrahydronaphthalen-2-amine (100 mg, 0.39 mmol) and 2,3-dichlorobenzenesulfonyl chloride (100 mg, 0.41 nm mot) were suspended in dichloromethane and DIPEA was added dropwise until a clear solution was obtained. The mixture was stirred for 1 h. MP-Trisamine (400 mg) was added and the mixture was stirred for 3 h. The mixture was filtered and the solvent was evaporated. The residue was purified by column chromatography on silica eluting with gradients of methanol in chloroform to give a solid (130 mg, 71%).

¹H NMR (600 MHz, CDCl₃) δ ppm 8.10 (1H, dd) 7.72 (1H, dd) 7.39-7.42 (1H, m) 7.02 (1H, d) 6.90 (1H, d) 5.19 (1H, d) 3.60-3.67 (1H, m) 2.88-3.14 (7H, m) 2.69-2.77 (2H, m) 2.57-2.68 (6H, m) 2.16 (3H, s) 1.95-2.02 (1H, m) 1.76-1.84 (1H, m); ESI-MS m/z M+H⁺ 468, 470.

### Example 12

### N-[(2R)-8-(4-Methylpiperazin-1-yl)-1,2,3,4-tetrahydronaphthalen-2-yl]naphthalene-2-sulfonamide

(2R)-5-Methyl-g-(4-methylpiperazin-1-yl)-1,2,3,4-tetrahydronaphthalen-2-amine (70 mg, 0.29 mmol) and naphtalene-2-sulfonyl chloride (70 mg, 0.31 mmol) were dissolved in dichloromethane (3 ml) and DIPEA (1 ml) was added. The mixture was stirred at ambient temperature for 20 h. The solvent was evaporated and the residue was dissolved in dichloromethane. The mixture was washed with saturated aqueous ammonium chloride, dried (Na₂SO₄), filtered and the solvent was evaporated. The residue was purified by column chromatography on silica eluting with gradients of 2% ammonia in methanol and chloroform to give a solid (86 mg, 68%).

¹H NMR (400 MHz, DMSO-d₆) δ ppm 8.48 (1H, s) 8.15 (2H, d) 8.05 (1H, d) 7.95 (1H, br. s.) 7.91 (1H, dd) 7.63-7.73 (2H, m) 7:01 (1H, t) 6.71-6.76 (1H, m) 3.40 (1H, br. s.) 2.59-2.84 (6H, m) 2.37-2.45 (2H, m) 2.19-2.29 (1H, m) 2.01 (3H, s) 1.99 (3H, br. s.) 1.86 (1H, br. s.) 1.55-1.67 (1H, m); ESI-MS m/z M+H⁺ 436.

### Examples 13

### 3-chloro-4-methyl-N-[(2R)-8-(4-methylpiperazin-1-yl)-1,2,3,4-tetrahydronaphthalen-2-yl]benzenesulfonamide

The title compound was prepared according to the method described in Example 12. The yield was 60%.

¹H NMR (400 MHz, DMSO-d₆) δ ppm 7.93 (1H, br. s.) 7.85 (1H, d) 7.72 (1H, dd) 7.59 (1H, d) 7.04 (1H, t) 6.81 (1H, d) 6.76 (1H, d) 2.70-2.85 (5H, m) 2.46-2.60 (4H, m) 2.43 (3H, s) 2.22-2.32 (4H, m) 2.21 (3H, s) 1.82-1.89 (1H, m) 1.55-1.66 (1H, m) ESI-MS m/z M+H⁺ 434, 436.

### Examples 14

### N-[(2R)-5-methyl-8-(4-methylpiperazin-1-yl)-1,2,3,4-tetrahydronaphthalen-2-yl]naphthalene-2-sulfonamide

The title compound was prepared according to the method described in Example 12. The yield was 79%.

¹H NMR (500 MHz, DMSO-d₆) δ ppm 8.47 (1H, s) 8.15 (2H, d) 8.06 (1H, d) 7.94 (1H, d) 7.91 (1H, d) 7.72 (1H, dd) 7.67 (1H, dd) 6.89 (1H, d) 6.70 (1H, d) 3.05-2.98 (1H, m) 2.95-2.10 (11H, m) 2.06 (3H, s) 2.02 (3H, s) 1.95-1.88 (2H, m) 1.68-1.57 (1H, m) ESI-MS m/z M+H⁺ 450.

### Example 15

### 1-[3-chloro-5-(trifluoromethyl)pyridin-2-yl]-N-[(2R)-5-methyl-8-(4-methylpiperazin-1-yl)-1,2,3,4-tetrahydronaphthalen-2-yl]-1H-pyrrole-2-sulfonamide

The title compound was prepared according to the method described in Example 12. The yield was 64%.

¹H NMR (400 MHz, CDCl₃) δ ppm 8.69-8.71 (1H, m) 8.15 (1H, d) 8.08 (1H, dd) 7.59 (1H; dd) 7.01 (1H, d) 6.87 (1H, d) 6.67 (1H, dd) 4.50 (1H, d) 3.70-3.79 (1H, m) 3.06 (1H, dd) 2.61-2.90 (7H, m) 2.51 (4H, br. s.) 2.32 (3H, s) 2.17 (3H, s) 2.02-2.11 (1H, m) 1.82-1.93 (1H, m) ESI-MS m/z M+H⁺ 568, 570.

### Example 16

### 5-Chloro-N-[(2R)-5-methyl-8-piperazin-1-yl-1,2,3,4-tetrahydronaphthalen-2-yl]naphthalene-2-sulfonamide

tert-Butyl 4-[(7R)-7-amino-4-methyl-5,6,7,8-tetrahydronaphthalen-1-yl]piperazine-1-carboxylate (55 mg, 0.16 mmol) and 5-chloronaphthalene-2-sulfonyl chloride (45 mg, 0.17 mmol) were dissolved in dichloromethane (3 ml) and DIPEA (1 ml) was added. The mixture was stirred at ambient temperature for 20 h. The solvent was evaporated and the residue was dissolved in dichloromethane. The mixture was washed with saturated aqueous ammonium chloride, dried (Na₂SO₄), filtered and the solvent was evaporated. The residue was purified by column chromatography on silica eluting with gradients of EtOAc and heptane to give a solid. ESI-MS m/z M+H⁺ 570, 572. The obtained solid was dissolved in dichloromethane (3 ml) and TFA (1 ml) was added. The mixture was stirred at ambient temperature for 5 h. The solvent was evaporated and the residue was dissolved in dichloromethane and washed with saturated aqueous sodium hydrogen carbonate. The organic phase was dried (Na₂SO₄), filtered and the solvent was evaporated. The residue was purified by column chromatography on silica eluting with gradients of 3% ammonia in methanol and chloroform to give a solid (36 mg, 48%).

¹H NMR (400 MHz, CDCl₃) δ ppm 8.49 (1H, s) 8.43 (1H, d) 7.99 (1H, dd) 7.90 (1H, d) 7.75 (1H, d) 7.54 (1H, t) 6.98 (1H, d) 6.79 (1H, d) 3.69-3.78 (1H, m) 2.43-2.93 (12H, m) 2.14 (3H, s) 1.97-2.07 (1H, m) 1.74-1.86 (1H, m); ESI-MS m/z M+H⁺ 470, 472.

### Example 17

### N-[(2R)-5-Methyl-8-piperazin-1-yl-1,2,3,4-tetrahydronaphthalen-2-yl]naphthalene-2-sulfonamide

N-[(2R)-5-Methyl-8-(4-methylpiperazin-1-yl)-1,2,3,4-tetrahydronaphthalen-2-yl]naphthalene-2-sulfonamide (200 mg, 0.44 mmol) and chloroethyl chloroformate (0.20 ml, 1.8 mmol) were dissolved in dichloroethane (3 ml). The mixture was heated at 140° C. for 10 min using microwave irradiation. Methanol (5 ml) was added and the mixture was stirred at ambient temperature for 20 h. The solvent was removed and the residue was dissolved in dichloromethane and the mixture was washed with saturated aqueous sodium hydrogen carbonate. The organic phase was dried (Na₂SO₄), filtered and the solvent was evaporated. The residue was purified by column chromatography on silica eluting with gradients of 3% ammonia in methanol and chloroform to give a solid (105 mg, 55%).

¹H NMR (400 MHz, CDCl₃) δ ppm 8.48 (1H, s) 7.93-8.02 (3H, m) 7.88 (1H, dd) 7.60-7.70 (2H, m) 6.99 (1H, d) 6.79 (1H, d) 4.62 (1H, br. s.) 3.76 (1H, br. s.) 2.89 (1H, dd) 2.55-2.75 (10H, m) 2.48 (1H, dd) 2.15 (3H, s) 1.96-2.05 (1H, m) 1.75-1.86 (1H, m); ESI-MS m/z M+H⁺ 436.

### Example 18

### 2,6-Dichloro-N-[(2R)-5-methyl-8-piperazin-1-yl-1,2,3,4-tetrahydronaphthalen-2-yl]benzenesulfonamide

tert-Butyl 4-[(7R)-7-amino-4-methyl-5,6,7,8-tetrahydronaphthalen-1-yl]piperazine 1-carboxylate (87 mg, 0.25 mmol) was dissolved in dichloromethane (2 ml) and DIPEA (52 μl, 0.30 mmol) and 2,6-dichlorobenzenesulfonyl chloride (68 mg, 0.28 mmol) were added. The mixture was stirred for 4 h. The solvent was evaporated and the residue was dissolved in dichloromethane (2 ml) and TFA (0.24 ml) was added. The mixture was stirred at ambient temperature for 15 h. The mixture was concentrated and EtOAc was added. The mixture was washed with aqueous sodium hydroxide (pH 8-9), dried (Na₂SO₄), filtered and the solvent was evaporated. The residue was purified by preparative HPLC to give a dry film (6.5 mg, 5%).

¹H NMR (400 MHz, MeOD-d₄) δ ppm 7.58 (2H, d) 7.48 (1H, d) 6.97 (1H, d) 6.81 (1H, d) 3.53-3.61 (1H, m) 3.01-3.17 (5H, m) 2.91-2.98 (2H, m) 2.74-2.88 (3H, m) 2.54-2.63 (2H, m) 2.14 (3H, s) 1.96-2.04 (1H, m) 1.74-1.85 (1H, m); ESI-MS m/z M+H⁺ 454, 456, 458.

Examples 19-27 were prepared according to the method described in Example 18.

| | | ¹H NMR | ESI-MS | |
|---|---|---|---|---|
| Ex. | | (400 MHz, MeOD-d₄) δ | m/z | |
| no | Compound Name | ppm | M + H⁺ | Yield % |
| 19 | 3,5-dimethyl-N-[(2R)-5-methyl- | 7.00 (1H, d) 6.85 (1H, d) | 405 | 35 |
| | 8-piperazin-1-yl-1,2,3,4- | 3.49-3.58 (1H, m) 3.22 (4H, | | |
| | tetrahydronaphthalen-2-yl] | br. s.) 2.78-3.02 (6H, | | |
| | isoxazole-4-sulfonamide | m) 2.63-2.73 (1H, m) | | |
| | | 2.59 (3H, s) 2.48 (1H, dd) | | |
| | | 2.33 (3H, s) 2.17 (3H, s) | | |
| | | 2.00-2.09 (1H, m) | | |
| | | 1.77-1.87 (1H, m) | | |
| 20 | 2-chloro-6-methyl-N-[(2R)-5- | 7.49 (1H, d) 7.40-7.45 (1H, | 434, 436 | 29 |
| | methyl-8-piperazin-1-yl-1,2,3,4- | m) 7.34 (1H, d) 6.97 (1H, | | |
| | tetrahydronaphthalen-2- | t) 6.81 (1H, t) | | |
| | yl]benzenesulfonamide | 3.43-3.51 (1H, m) 3.14 (3H, br. | | |
| | | s.) 2.94-3.01 (2H, m) | | |
| | | 2.78-2.88 (2H, m) 2.71 (3H, | | |
| | | s) 2.49-2.63 (2H, m) | | |
| | | 2.14 (3H, s) 1.95-2.03 (1H, | | |
| | | m) 1.71-1.82 (1H, m) | | |
| 21 | 2,6-difluoro-N-[(2R)-5-methyl-8- | 7.61-7.70 (1H, m) 7.16 (2H, | 422 | 26 |
| | piperazin-1-yl-1,2,3,4-tetrahydro | t) 6.98 (1H, d) 6.85 (1H, | | |
| | naphthalen-2-yl]benzenesulfonamide | d) 3.54-3.63 (1H, m) | | |
| | | 3.18 (5H, br. s.) | | |
| | | 2.97-3.06 (2H, m) 2.76-2.94 (3H, | | |
| | | m) 2.54-2.70 (2H, m) | | |
| | | 2.15 (3H, s) 1.99-2.08 (1H, | | |
| | | m) 1.71-1.84 (1H, m) | | |
| 22 | N-[(2R)-5-methyl-8-piperazin-1- | 9.42 (1H, s) 8.63 (1H, d) | 437 | 7 |
| | yl-1,2,3,4-tetrahydronaphthalen- | 8.59 (1H, d) 8.51 (1H, dd) | | |
| | 2-yl]isoquinoline-5-sulfonamide | 8.40 (1H, d) 7.79-7.85 (1H, | | |
| | | m) 6.90 (1H, d) 6.74 (1H, | | |
| | | d) 3.42-3.50 (1H, m) | | |
| | | 2.95 (4H, br. s.) | | |
| | | 2.63-2.85 (6H, m) 2.45-2.55 (1H, | | |
| | | m) 2.37 (1H, dd) | | |
| | | 2.08 (3H, s) 1.84 (1H, br. s.) | | |
| | | 1.63-1.71 (1H, m) | | |
| 23 | 5-chloro-1,3-dimethyl-N-[(2R)-5- | 7.01 (1H, d) 6.87 (1H, d) | 438, 440 | 8 |
| | methyl-8-piperazin-1-yl-1,2,3,4- | 3.82 (3H, s) 3.43-3.51 (1H, | | |
| | tetrahydronaphthalen-2-yl]-1H- | m) 3.25 (4H, br. s.) | | |
| | pyrazole-4-sulfonamide | 3.00-3.10 (3H, m) 2.93 (2H, | | |
| | | br. s.) 2.78-2.87 (1H, | | |
| | | m) 2.52-2.67 (2H, m) | | |
| | | 2.40 (3H, s) 2.18 (3H, s) | | |
| | | 1.98-2.06 (1H, m) | | |
| | | 1.72-1.83 (1H, m) | | |
| 24 | 2,4-dimethyl-N-[(2R)-5-methyl- | 7.00 (1H, d) 6.87 (1H, d) | 421 | 12 |
| | 8-piperazin-1-yl-1,2,3,4- | 3.47-3.56 (1H, m) 3.21 (4H, | | |
| | tetrahydronaphthalen-2-yl]-1,3- | br. s.) 2.98-3.08 (3H, | | |
| | thiazole-5-sulfonamide | m) 2.76-2.95 (3H, m) | | |
| | | 2.69 (3H, s) 2.56 (3H, s) | | |
| | | 2.48-2.66 (2H, m) 2.16 (3H, | | |
| | | s) 1.97-2.05 (1H, m) | | |
| | | 1.70-1.82 (1H, m) | | |
| 25 | 1,3,5-trimethyl-N-[(2R)-5- | 6.88 (1H, d) 6.75 (1H, d) | 418 | 2 |
| | methyl-8-piperazin-1-yl-1,2,3,4- | 3.64 (3H, s) 3.11 (4H, br. | | |
| | tetrahydronaphthalen-2-yl]-1H- | s.) 2.86-2.95 (3H, m) | | |
| | pyrazole-4-sulfonamide | 2.65-2.82 (3H, m) | | |
| | | 2.45-2.55 (1H, m) 2.35-2.44 (1H, | | |
| | | m) 2.32 (3H, s) 2.25 (3H, | | |
| | | s) 2.05 (3H, s) | | |
| | | 1.85-1.93 (1H, m) 1.58-1.69 (1H, | | |
| | | m) | | |
| 26 | 4-bromo-2,5-dichloro-N-[(2R)-5- | 6.99 (1H, d) 6.85 (1H, d) | 538, 540, | 34 |
| | methyl-8-piperazin-1-yl-1,2,3,4- | 3.51-3.60 (1H, m) 3.21 (4H, | 542, 544 | |
| | tetrahydronaphthalen-2- | br. s.) 2.96-3.10 (3H, | | |
| | yl]thiophene-3-sulfonamide | m) 2.76-2.95 (3H, m) | | |
| | | 2.57-2.69 (2H, m) 2.16 (3H, | | |
| | | s) 1.99-2.08 (1H, m) | | |
| | | 1.75-1.88 (1H, m) | | |
| 27 | 4-bromo-N-[(2R)-5-methyl-8- | 7.77 (1H, d) 7.23 (1H, d) | 470, 472 | 21 |
| | piperazin-1-yl-1,2,3,4- | 6.99 (1H, d) 6.85 (1H, d) | | |
| | tetrahydronaphthalen-2- | 3.47-3.57 (1H, m) | | |
| | yl]thiophene-3-sulfonamide | 3.22 (4H, br. S.) 3.00-3.15 (3H, | | |
| | | m) 2.77-2.93 (3H, m) | | |
| | | 2.49-2.67 (2H, m) | | |
| | | 2.15 (3H, s) 2.00-2.08 (1H, | | |
| | | m) 1.71-1.82 (1H, m) | | |

### Example 28

### N-[4-Methyl-5-({[(2R)-5-methyl-8-piperazin-1-yl-1,2,3,4-tetrahydronaphthan-2-yl]amino}sulfonyl)-1,3-thiazol-2-yl]acetamide

tert-Butyl 4-[(7R)-7-amino-4-methyl-5,6,7,8-tetrahydronaphthalen-1-yl]piperazine-1-carboxylate (87 mg, 0.25 mmol) was dissolved in dichloromethane (2 ml) and DIPEA (0.36 ml, 2.0 mmol) was added followed by 2-(acetylamino)-4-methyl-1,3-thiazole-5-sulfonyl chloride (70 mg, 0.28 mmol). The mixture was stirred at ambient temperature for 1.5 h. Triethylsilyl chloride (0.38 ml, 3.0 mmol) and lithium iodide (400 mg, 3.0 mmol) were stirred for 1.5 h and then added to the reaction mixture. The resulting mixture was stirred an additional 1.5 h at ambient temperature. Methanol (2 ml) and DIPEA (0.6 ml) were added. The solvent was evaporated and the residue was purified by preparative HPLC to give the acetate of the title compound as a solid (28 mg, 21%).

¹H NMR (400 Mz, MeOD-d₄) δ ppm 6.99 (1H, d) 6.86 (1H, d) 3.45-3.54 (1H, m) 3.20-3.26 (4H, m) 3.11-3.19 (1H, m) 2.97-3.05 (2H, m) 2.76-2.94 (3H, m) 2.51-2.68 (2H, m) 2.48 (3H, s) 2.23 (3H, s) 2.16 (3H, s) 1.99-2.07 (1H, m) 1.71-1.82 (1H, m); ESI-MS m/z M+H⁺ 464.

### Example 29

### 2,4-Dimethyl-N-[(2R)-5-methyl-8-(4-methylpiperazin-1-yl)-1,2,3,4-tetrahydronaphthalen-2-yl]-1,3-thiazole-5-sulfonamide

(2R)-5-Methyl-8-(4-methylpiperazin-1-yl)-1,2,3,4-tetrahydronaphthalen-2-amine (50 mg, 0.20 mmol) was dissolved in dichloromethane (3 ml) and DIPEA (105 μl, 0.60 mmol) was added. 2,4-Dimethyl-1,3-thiazole-5-sulfonyl chloride (63 mg, 0.30 mmol) was added slowly and the mixture was stirred at ambient temperature for 40 min. The solvent was evaporated and the residue was purified by column chromatography on silica eluting with ammonia in methanol and chloroform to give a solid (69 mg, 72%).

¹H NMR (400 MHz, CDCl₃) δ ppm 7.03 (1H, d) 6.89 (1H, d) 4.71 (1H, d) 3.74-3.82 (1H, m) 2.98 (1H, dd) 2.87 (4H, br. s.) 2.59-2.73 (8H, m) 2.58 (3H, s) 2.43 (4H, br. s.) 2.18 (3H, s) 1.98-2.07 (1H, m) 1.82-1.92 (1H, m); ESI-MS m/z M+H⁺ 435.

### Example 30

### N-[(2R)—S-Methyl-8-(4-methylpiperazin-1-yl)-1,2,3,4-tetrahydronaphthalen-2-yl]-1,3-benzothiazole-6-sulfonamide

The title compound was prepared according to the method in example 29.

¹H NMR (400 MHz, CDCl₃) δ ppm 9.23 (1H, s) 8.60 (1H, d) 8.28 (1H, d) 8.04 (1H, dd) 6.99 (1H, d) 6.85 (1H, d) 4.79 (1H, d) 3.68-3.78 (1H, m) 2.57-2.98 (11H, m) 2.41-2.56 (4H, m) 2.15 (3H, s) 1.93-2.02 (1H, m) 1.74-1.84 (1H, m); ESI-MS m/z M+H⁺ 457.

### Example 31

### 5-Chloro-N-[(2R)-8-piperazin-1-yl-1,2,3,4-tetrahydronaphthalen-2-yl]naphthalene-2-sulfonamide

tert-Butyl 4-[(7R)-7-amino-5,6,7,8-tetrahydronaphthalen-1-yl]piperazine-1-carboxylate (250 mg, 0.75 mmol), 5-chloronaphthalene-2-sulfonyl chloride (193 mg, 0.74 mmol) and is pyridine (90 μl, 1.1 mmol) were stirred at ambient temperature in dichloromethane (1 ml) for 22 h. DIPEA (126 μl, 0.74 mmol) was added and the mixture was stirred at ambient temperature for 15 min. The solid formed was collected by filtration and washed with dichloromethane to give a solid (147 mg). The filtrate was washed with aqueous citric acid, water and aqueous saturated sodium hydrogen carbonate. The organic phase was dried (Na₂SO₄) and the solvent was evaporated. The residue was recrystallized from EtOAc and hexane to give additional solid (183 mg). The combined solids (325 mg) were dissolved in dichloromethane (1 ml) and TFA (1 ml) was added. The mixture was stirred at ambient temperature for 45 min. The solvents were evaporated and the residue was dissolved in DMF and methanol and piperazine (0.5 mmol) was added. The mixture was purified by preparative HPLC to give the acetate of the title compound as solid (152 mg, 57%).

¹H NMR (400 MHz, CDCl₃) δ ppm 8.50 (1H, s) 8.35-8.45 (1H, m) 7.96-8.06 (1H, m) 7.84-7.93 (1H, m) 7.69-7.78 (1H, m) 7.48-7.58 (1H, m) 7.01-7.13 (1H, m) 6.75-6.86 (2H, m) 3.61 (1H, m) 2.61-3.14 (10H, m) 2.40-2.61 (1H, m) 2.20-2.39 (1H, m) 1.90-2.03 (1H, m) 1.63-1.84 (1H, m); ESI-MS m/z M+H⁺ 456, 458.

### Example 32

### 4-Chloro-N-[(2R)-8-piperazin-1-yl-1,2,3,4-tetrahydronaphthalen-2-yl]naphthalene-1-sulfonamide

tert-Butyl 4-[(7R)-7-amino-5,6,7,8-tetrahydronaphthalen-1-yl]piperazine-1-carboxylate (51 mg, 0.15 mmol), 4-chloronaphthalene-1-sulfonyl chloride (40 mg, 0.15 mmol) and pyridine (18 μl, 0.23 mmol) were stirred at ambient temperature in dichloromethane (1 ml) for 4 h. DIPEA (26 μl, 0.15 mmol) was added and the mixture was stirred at ambient temperature for 20 h. The solvent was evaporated and the mixture was purified by column chromatography on silica eluting with gradients of EtOAc and heptane to give a solid (77 mg). The solid was dissolved in dichloromethane (1 ml) and TFA (1 ml) was added. The mixture was stirred for 1.5 h. Toluene (1 ml) was added and the solvents were evaporated. The residue was purified by preparative HPLC to give the acetate of the title compound as a solid (64 mg, 93%).

¹H NMR (400 MHz, DMSO-d₆) δ ppm 8.81 (1H, m) 8.40 (1H, m) 8.16 (1H, d) 7.82-7.89 (3H, m) 6.99 (1H, t) 6.66-6.72 (2H, m) 3.29 (1H, m) 2.26-2.83 (11H, m) 2.13 (1H, m) 1.73-1.83 (1H, m) 1.49-1.61 (1H, m); ESI-MS m/z M+H⁺ 456, 458.

### Example 33

### 5-Chloro-N-[(2R)-8-piperazin-1-yl-1,2,3,4-tetrahydronaphthalen-2-yl]naphthalene-1-sulfonamide

The title compound was prepared according to the method described in Example 32.

¹H NMR (400 MHz, CDCl₃) δ ppm 8.64 (1H, d) 8.57 (1H, d) 8.38 (1H, d) 7.73 (1H, d) 7.62-7.69 (1H, m) 7.52-7.59 (1H, m) 7.01-7.09 (1H, m) 6.71-6.82 (2H, m) 3.47-3.57 (1H, m) 2.52-3.17 (m, 11H) 2.41 (1H, m) 1.74-1.86 (1H, m) 1.49-1.64 (1H, m) ESI-MS m/z M+H⁺ 456, 458.

### Example 34

### 1-(3-Chlorophenyl)-N-[(2R)-8-piperazin-1-yl-1,2,3,4-tetrahydronaphthalen-2-yl]methanesulfonamide

The title compound was prepared according to the method described in Example 32, but no pyridine was added and 2.5 eq of DIPEA was used.

¹H NMR (400 MHz, CDCl₃) δ ppm 7.28-7.38 (4H, m) 7.16 (1H, t) 6.93 (1H, d) 6.90 (I H, d) 4.21-4.29 (2H, m) 3.57-3.65 (1H, m) 3.18-3.26 (4H, m) 3.08 (1H, dd) 2.95-3.04 (4H, m) 2.84-2.92 (2H, m) 2.66 (1H, dd) 2.02-2.10 (1H, m) 1.71-1.83 (1H, m) ESI-MS m/z M+H⁺ 420, 422.

### Example 35

### 2-(4-Chlorophenyl)-N-[(2R)-8-piperazin-1-yl-1,2,3,4-tetrahydronaphthalen-2-yl]ethanesulfonamide

The title compound was prepared according to the method described in Example 32 but no pyridine was added and 2.5 eq of DIPEA was used.

¹H NMR (400 MHz, CDCl₃) δ ppm 7.26-7.32 (2H, m) 7.11-7.20 (3H, m) 6.88-6.94 (2H, m) 3.77-3.86 (1H, m) 3.26-3.33 (2H, m) 3.05-3.16 (7H, m) 2.89-2.96 (6H, m) 2.70 (1H, dd) 2.05-2.14 (1H, m) 1.77-1.88 (1H, m) ESI-MS m/z M+H⁺ 434, 436.

### Example 36

### N-[(2R)-5-Methyl-8-(4-methylpiperazin-1-yl)-1,2,3,4-tetrahydronaphthalen-2-yl]benzenesulfonamide

To a solution of (2R)-5-methyl-8-(4-methylpiperazin-1-yl)-1,2,3,4-tetrahydronaphthalen-2-amine (25.9 mg, 0.10 mmol) and triethylamine (42 ul, 0.30 mmol) in N-methylpyrrolidinone (0.5 ml) was added benzenesulfonyl chloride (0.10 mmol). The reaction was shaken for 18 hours at ambient temperature. The volatiles were removed under vacuum and the crude was dissolved in methanol and purified on Tosic-65 resin conditioned with methanol and the compound was eluted with a 1N solution of ammonia in methanol. The solvents were removed under vacuum and the crude material was dissolved in DMSO and purified by reverse phase HPLC to give the title compound.

¹H NMR (500 MHz, DMSO-d₆) δ 7.86 (2H, d), 7.81 (1H, d), 7.67 (1H, dd), 7.63 (2H, dd), 6.93 (1H, d), 6.75 (1H, d), 3.10-2.90 (1H, m), 2.90-2.10 (11H, m), 2.23 (3H, s), 2.07 (1H, s), 1.90-1.82 (2H, m), 1.65-1.55 (1H, m); ESI-MS m/z M+H⁺ 400.

Examples 37-61 were prepared using the method described in Example 36.

| | | ESI-MS | ¹H NMR |
|---|---|---|---|
| Ex. | Compound | m/z | (500 MHz, |
| no | Name | M + H⁺ | DMSO-d₆) |
| 37 | 3-Cyano-N-[(2R)-5-methyl-8-(4- | 425 | 8.28 (1H, d) 8.16 (1H, |
| | methylpiperazin-1-yl)-1,2,3,4- | | dd) 8.06 (1H, d) 7.85 (1H, |
| | tetrahydronaphthalen-2-yl]benzene | | t) 6.94 (1H, d) |
| | sulfonamide | | 6.75 (1H, d) 3.01-2.95 (1H, |
| | | | m) 2.85-2.20 (11H, m) |
| | | | 2.23 (3H, s) 2.08 (3H, |
| | | | s) 1.95-1.85 (2H, m) |
| | | | 1.68-1.58 (1H, m). |
| 38 | 5-Chloro-1,3-dimethyl-N-[(2R)-5- | 452 | 7.86 (1H, d) 6.94 (1H, |
| | methyl-8-(4-methylpiperazin-1-yl)- | | d) 6.78 (1H, d) 3.78 (3H, |
| | 1,2,3,4-tetrahydronaphthalen-2-yl]- | | s) 2.92 (1H, dd) |
| | 1H-pyrazole-4-sulfonamide | | 2.85-2.20 (11H, m) |
| | | | 2.32 (3H, s) 2.24 (3H, s) |
| | | | 2.09 (3H, s) |
| | | | 1.95-1.88 (2H, m) 1.70-1.60 (1H, |
| | | | m) |
| 39 | 3,5-Dimethyl-N-[(2R)-5-methyl-8-(4- | 419 | 8.11 (1H, d) 6.94 (1H, |
| | methylpiperazin-1-yl)-1,2,3,4- | | d) 6.78 (1H, d) |
| | tetrahydronaphthalen-2-yl]isoxazole- | | 2.95-2.91 (1H, m) 2.74 (3H, |
| | 4-sulfonamide | | s) 2.80-2.10 (11H, m) |
| | | | 2.33 (3H, s) 2.24 (3H, |
| | | | s) 2.09 (3H, s) |
| | | | 1.96-1.89 (2H, m) |
| | | | 1.73-1.65 (1H, m) |
| 40 | 2,6-Difluoro-N-[(2R)-5-methyl-8-(4- | 436 | 8.44 (1H, d) |
| | methylpiperazin-1-yl)-1,2,3,4- | | 7.77-7.50 (1H, m) 7.32 (2H, t) |
| | tetrahydronaphthalen-2- | | 6.93 (1H, d) 6.77 (1H, |
| | yl]benzenesulfonamide | | d) 2.95-2.92 (1H, m) |
| | | | 2.80-2.10 (11H, m) |
| | | | 2.24 (3H, s) 2.09 (3H, s) |
| | | | 1.98-1.82 (2H, m) |
| | | | 1.73-1.65 (1H, m) |
| 41 | 2-Chloro-4-fluoro-N-[(2R)-5-methyl- | 452 | 8.12 (1H, d) 8.08 (1H, |
| | 8-(4-methylpiperazin-1-yl)-1,2,3,4- | | dd) 7.73 (1H, dd) |
| | tetrahydronaphthalen-2- | | 7.42 (1H, dd) 6.91 (1H, d) |
| | yl]benzenesulfonamide | | 6.74 (1H, d) |
| | | | 3.05-2.95 (1H, m) 2.80-2.15 (11H, |
| | | | m) 2.24 (3H, s) |
| | | | 2.07 (3H, s) 1.98-1.85 (2H, |
| | | | m) 1.75-1.65 (1H, m) |
| 42 | N-[(2R)-5-Methyl-8-(4- | 468 | 8.19 (1H, d) 8.05 (1H, |
| | methylpiperazin-1-yl)-1,2,3,4- | | d) 8.02 (1H, d) |
| | tetrahydronaphthalen-2-yl]-2- | | 7.90-7.85 (2H, m) 6.92 (1H, |
| | (trifluoromethyl)benzenesulfonamide | | d) 6.74 (1H, d) |
| | | | 3.05-2.95 (1H, m) |
| | | | 2.90-2.10 (11H, m) 2.21 (3H, s) |
| | | | 2.08 (3H, s) |
| | | | 1.98-1.88 (2H, m) 1.75-1.65 (1H, |
| | | | m) |
| 43 | 5-Chloro-2-methoxy-N-[(2R)-5- | 464 | 7.70 (1H, d) 7.68 (1H, |
| | methyl-8-(4-methylpiperazin-1-yl)- | | dd) 7.58 (1H, d) 7.31 (1H, |
| | 1,2,3,4-tetrahydronaphthalen-2- | | d) 6.92 (1H, d) |
| | yl]benzenesulfonamide | | 6.75 (1H, d) 3.91 (3H, s) |
| | | | 3.05-2.95 (1H, m) |
| | | | 2.90-2.22 (11H, m) 2.24 (3H, |
| | | | s) 2.08 (3H, s) |
| | | | 1.95-1.87 (2H, m) |
| | | | 1.72-1.65 (1H, m) |
| 44 | N-[(2R)-5-Methyl-8-(4- | 445 | 8.47 (2H, d) 8.25 (1H, |
| | methylpiperazin-1-yl)-1,2,3,4-tetra | | d) 8.13 (2H, d) 6.93 (1H, |
| | hydronaphthalen-2-yl]-4- | | d) 6.75 (1H, d) |
| | nitrobenzenesulfonamide | | 3.07-2.95 (1H, m) |
| | | | 2.80-2.10 (11H, m) 2.12 (3H, |
| | | | s) 2.08 (3H, s) |
| | | | 2.00-1.90 (2H, m) |
| | | | 1.70-1.60 (1H, m) |
| 45 | N-[(2R)-5-Methyl-8-(4- | 414 | 7.38 (5H, m) 7.17 (1H, |
| | methylpiperazin-1-yl)-1,2,3,4-tetra | | d) 6.96 (1H, d) 6.82 (1H, |
| | hydronaphthalen-2-yl]-1- | | d) 4.36 (2H, s) |
| | phenylmethanesulfonamide | | 3.14 (1H, dd) 2.95-2.15 (11H, |
| | | | m) 2.25 (3H, s) |
| | | | 2.12 (3H, s) 2.10-2.00 (2H, |
| | | | m) 1.68-1.57 (1H, m) |
| 46 | 4-Fluoro-N-[(2R)-5-methyl-8-(4- | 418 | 7.92 (2H, dd) 7.86 (2H, |
| | methylpiperazin-1-yl)-1,2,3,4- | | d) 7.45 (1H, dd) 6.92 (1H, |
| | tetrahydronaphthalen-2- | | d) 6.75 (1H, d) |
| | yl]benzenesulfonamide | | 3.05-2.95 (1H, m) |
| | | | 2.85-2.15 (11H, m) 2.22 (3H, s) |
| | | | 2.08 (3H, s) |
| | | | 1.95-1.85 (2H, m) 1.65-1.58 (1H, |
| | | | m) |
| 47 | N-[4-({[(2R)-5-Methyl-8-(4- | 457 | 10.35 (1H, s) 7.78 (4H, |
| | methylpiperazin-1-yl)-1,2,3,4- | | m) 7.67 (1H, dd) |
| | tetrahydronaphthalen-2-yl]amino} | | 6.92 (1H, d) 6.75 (1H, |
| | sulfonyl)phenyl]acetamide | | d) 3.05-2.95 (1H, m) |
| | | | 2.85-2.15 (11H, m) |
| | | | 2.19 (3H, s) 2.09 (3H, s) |
| | | | 2.07 (3H, s) |
| | | | 1.93-1.85 (2H, m) 1.63-1.56 (1H, |
| | | | m) |
| 48 | 2,5-Difluoro-N-[(2R)-5-methyl-8-(4- | 436 | 8.31 (1H, d) |
| | methylpiperazin-1-yl)-1,2,3,4-tetra | | 7.65-7.52 (3H, m) 6.94 (1H, d) |
| | hydronaphthalen-2-yl]benzenesulfonamide | | 6.76 (1H, d) |
| | | | 3.03-2.92 (1H, m) 2.85-2.20 (11H, |
| | | | m) 2.23 (3H, s) |
| | | | 2.08 (3H, s) 1.97-1.85 (2H, |
| | | | m) 1.73-1.61 (1H, m) |
| 49 | Methyl 1-metbyl-5-({[(2R)-5-methyl- | 461 | 7.64 (1H, s) 7.48 (d, 1H) |
| | 8-(4-methylpiperazin-1-yl)-1,2,3,4- | | 7.08 (1H, s) 6.94 (1H, |
| | tetrahydronaphthalen-2- | | d) 6.77 (1H, d) |
| | yl]amino}sulfonyl)-1H-pyrrole-2- | | 3.90 (3H, s) 3.78 (3H, s) |
| | carboxylate | | 2.92-2.85 (1H, m,) |
| | | | 2.80-2.20 (11H, m) |
| | | | 2.20 (3H, s) 2.08 (3H, |
| | | | s) 2.00-1.90 (2H, m) |
| | | | 1.65-1.55 (1H, m) |
| 50 | 4-Chloro-2-fluoro-N-[(2R)-5-methyl- | 451 | 8.27 (1H, d) 7.85 (1H, |
| | 8-(4-methylpiperazin-1-yl)-1,2,3,4- | | dd) 7.75 (1H, d) 7.50 (1H, |
| | tetrahydronaphthalen-2- | | d) 6.93 (1H, d) |
| | yl]benzenesulfonamide | | 6.75 (1H, d) 3.10-2.95 (1H, |
| | | | m) 2.92-2.18 (11H, |
| | | | m) 2.24 (3H, s) 2.08 (3H, |
| | | | s) 1.98-1.91 (2H, |
| | | | m) 1.74-1.63 (1H, m) |
| 51 | 3,5-Dimethyl-N-[(2R)-5-methyl-8-(4- | 428 | 7.71 (1H, d) 7.46 (2H, |
| | methylpiperazin-1-yl)-1,2,3,4-tetra | | s) 7.28 (1H, s) 6.92 (1H, |
| | hydronaphthalen-2- | | d) 6.74 (1H, d) |
| | yl]benzenesulfonamide | | 3.05-2.95 (1H, m) |
| | | | 2.89-2.11 (11H, m) 2.37 (6H, |
| | | | s) 2.22 (3H, s) |
| | | | 2.07 (3H, s) 1.93-1.87 (2H, |
| | | | m) 1.64-1.56 (1H, m) |
| 52 | N-[(2R)-5-Methyl-8-(4- | 401 | 9.01 (1H, s) 8.84 (1H, |
| | methylpiperazin-1-yl)-1,2,3,4-tetra | | d) 8.23 (1H, dd) 7.67 (1H, |
| | hydronaphthalen-2-yl]pyridine-3- | | dd) 6.93 (1H, d) |
| | sulfonamide | | 6.76 (1H, d) 3.05-2.95 (1H, |
| | | | m) 2.88-2.17 (11H, |
| | | | m) 2.22 (3H, s) 2.08 (3H, |
| | | | s) 1.93-1.86 (2H, |
| | | | m) 1.66-1.59 (1H, m) |
| 53 | 5-Bromo-2,4-difluoro-N-[(2R)-5- | 515 | 8.38 (1H, d) 8.09 (1H, |
| | methyl-8-(4-methylpiperazin-1-yl)- | | t) 7.84 (1H, t) 6.93 (1H, |
| | 1,2,3,4-tetrahydronaphthalen-2-yl] | | d) 6.76 (1H, d) |
| | benzenesulfonamide | | 3.05-2.95 (1H, m) |
| | | | 2.87-2.18 (11H, m) 2.22 (3H, |
| | | | s) 2.08 (3H, s) |
| | | | 1.99-1.92 (2H, m) |
| | | | 1.75-1.63 (1H, m) |
| 54 | 5-Bromo-6-chloro-N-[(2R)-5-methyl- | 513 | 8.83 (1H, s) 8.59 (1H, |
| | 8-(4-methylpiperazin-1-yl)-1,2,3,4- | | s) 8.25 (1H, d) 6.94 (1H, |
| | tetrahydronaphthalen-2-yl]pyridine-3- | | d) 6.76 (1H, d) |
| | sulfonamide | | 3.10-3.00 (1, H, m) |
| | | | 2.87-2.18 (11H, m) 2.24 (3H, |
| | | | s) 2.09 (3H, s) |
| | | | 2.01-1.92 (m, 2H) |
| | | | 1.74-1.65 (1H, m) |
| 55 | N-[(2R)-5-Methyl-8-(4- | 456 | 8.47 (1H, s) 8.28 (1H, |
| | methylpiperazin-1-yl)-1,2,3,4- | | d) 8.14 (1H, t) |
| | tetrahydronaphthalen-2-yl]-1- | | 7.60-7.51 (2H, m) 6.89 (1H, |
| | benzothiophene-3-sulfonamide | | d) 6.70 (1H, d) |
| | | | 3.35-3.26 (1H, m) |
| | | | 2.86-2.15 (11H, m) 2.17 (3H, s) |
| | | | 2.05 (3H, s) |
| | | | 1.92-1.85 (2H, m) 1.67-1.56 (1H, |
| | | | m) |
| 56 | 4-Bromo-2,5-difluoro-N-[(2R)-5- | 515 | 8.41 (1H, d) 8.05 (1H, |
| | methyl-8-(4-methylpiperazin-1-yl)- | | dd) 7.77 (1H, t) 6.93 (1H, |
| | 1,2,3,4-tetrahydronaphthalen-2- | | d) 6.75 (1H, d) |
| | yl]benzenesulfonamide | | 3.05-2.95 (1H, m) |
| | | | 2.90-2.22 (11H, m) 2.24 (3H, |
| | | | s) 2.08 (3H, s) |
| | | | 1.99-1.92 (2H, m) |
| | | | 1.75-1.65 (1H, m) |
| 57 | 2,3,4-Trifluoro-N-[(2R)-5-methyl-8- | 454 | 8.44 (1H, d) |
| | (4-methylpiperazin-1-yl)-1,2,3,4-tetra | | 7.75-7.69 (1H, m) 7.58-7.49 (1H, |
| | hydronaphthalen-2- | | m) 6.92 (1H, d) |
| | yl]benzenesulfonamide | | 6.75 (1H, d) 3.05-2.95 (1H, |
| | | | m) 2.90-2.16 (11H, |
| | | | m) 2.21 (3H, s) 2.08 (3H, |
| | | | s) 2.00-1.93 (2H, |
| | | | m) 1.74-1.63 (1H, m) |
| 58 | N-[(2R)-5-Methyl-8-(4- | 406 | 8.15 (1H, d) 7.77 (1H, |
| | ethylpiperazin-1-yl)-1,2,3,4- | | dd) 7.73 (1H, d) 7.39 (1H, |
| | tetrahydronaphthalen-2-yl]thiophene- | | d) 6.93 (1H, d) |
| | 3-sulfonamide | | 6.77 (1H, d) 2.92-2.85 (1H, |
| | | | m) 2.80-2.20 (11H, m) |
| | | | 2.24 (3H, s) 2.08 (3H, |
| | | | s) 1.92-1.85 (2H, m) |
| | | | 1.65-1.55 (1H, m) |
| 59 | N-[(2R)-5-Methyl-8-(4- | 478 | 8.16 (2H, d) 8.15 (1H, |
| | methylpiperazin-1-yl)-1,2,3,4- | | d) 8.12 (2H, d) 6.93 (1H, |
| | tetrahydronaphthalen-2-yl]-4- | | d) 6.76 (1H, d) |
| | (methylsulfonyl)benzenesulfonamide | | 3.29 (3H, s) 2.92-2.85 (1H, |
| | | | m) 2.80-2.20 (11H, m) |
| | | | 2.21 (3H, s) 2.08 (3H, |
| | | | s) 1.92-1.85 (2H, s) |
| | | | 1.68-1.58 (1H, m) |
| 60 | 2-Chloro-4-cyano-N-[(2R)-5-methyl- | 459 | 8.42 (1H, d) 8.33 (1H, |
| | 8-(4-methylpiperazin-1-yl)-1,2,3,4- | | s) 8.17 (1H, d) 8.03 (1H, |
| | tetra | | d) 6.92 (1H, d) |
| | hydronaphthalen-2- | | 6.73 (1H, d) 3.05-2.95 (1H, |
| | yl]benzenesulfonamide | | m) 2.88-2.26 (11H, |
| | | | m) 2.25 (3H, s) 2.07 (3H, |
| | | | s) 1.98-1.90 (2H, |
| | | | m) 1.75-1.69 (1H, m) |
| 61 | 6-Chloro-N-[(2R)-5-methyl-8-(4- | 480 | 8.51 (1H, d) 8.00 (1H, |
| | methylpiperazin-1-yl)-1,2,3,4-tetra | | d) 7.61 (1H, d) 6.92 (1H, |
| | hydronaphthalen-2-yl]imidazo[2,1- | | d) 6.75 (1H, d) |
| | b][1,3]thiazole-5-sulfonamide | | 2.88-2.80 (1H, m) |
| | | | 2.80-2.10 (11H, m) 2.23 (3H, s) |
| | | | 2.07 (3H, s) |
| | | | 1.94-1.87 (2H, m) 1.72-1.64 (1H, |
| | | | m) |

Examples 62-151 were Prepared using the method described in Example 36.

| | | ESI-MS m/z |
|---|---|---|
| Example no | Compound Name | M + H⁺ |
| 62 | 4-Chloro-N-[(2R)-5-methyl-8-(4-methylpiperazin-1-yl)- | 434 |
| | 1,2,3,4-tetrahydronaphthalen-2-yl]benzenesulfonamide | |
| 63 | 3-Chloro-N-[(2R)-5-methyl-8-(4-methylpiperazin-1-yl)- | 434 |
| | 1,2,3,4-tetrahydronaphthalen-2-yl]benzenesulfonamide | |
| 64 | 1-(3-Chlorophenyl)-N-[(2R)-5-methyl-8-(4- | 448 |
| | methylpiperazin-1-yl)-1,2,3,4-tetrahydronaphthalen-2- | |
| | yl]methanesulfonamide | |
| 65 | N-[4-Methyl-5-({[(2R)-5-methyl-8-(4-methylpiperazin- | 478 |
| | 1-yl)-1,2,3,4-tetrahydronaphthalen-2- | |
| | yl]amino}sulfonyl)-1,3-thiazol-2-yl]acetamide | |
| 66 | N-[(2R)-5-Methyl-8-(4-methylpiperazin-1-yl)-1,2,3,4- | 406 |
| | tetrahydronaphthalen-2-yl]thiophene-2-sulfonamide | |
| 67 | N-[(2R)-5-Methyl-8-(4-methylpiperazin-1-yl)-1,2,3,4- | 445 |
| | tetrahydronaphthalen-2-yl]-3-nitrobenzenesulfonamide | |
| 68 | 2,5-Dimethoxy-N-[(2R)-5-methyl-8-(4-methylpiperazin- | 460 |
| | 1-yl)-1,2,3,4-tetrahydronaphthalen-2- | |
| | yl]benzenesulfonamide | |
| 69 | 5-Bromo-2-methoxy-N-[(2R)-5-methyl-8-(4- | 509 |
| | methylpiperazin-1-yl)-1,2,3,4-tetrahydronaphthalen-2- | |
| | yl]benzenesulfonamide | |
| 70 | N-[(2R)-5-Methyl-8-(4-methylpiperazin-1-yl)-1,2,3,4- | 546 |
| | tetrahydronaphthalen-2-yl]-4-(phenylsulfonyl)thiophene- | |
| | 2-sulfonamide | |
| 71 | N-[(2R)-5-Methyl-8-(4-methylpiperazin-1-yl)-1,2,3,4- | 546 |
| | tetrahydronaphthalen-2-yl]-5-(phenylsulfonyl)thiophene- | |
| | 2-sulfonamide | |
| 72 | N-[(2R)-5-Methyl-8-(4-methylpiperazin-1-yl)-1,2,3,4- | 458 |
| | tetrahydronaphthalen-2-yl]-2,1,3-benzothiadiazole-4- | |
| | sulfonamide | |
| 73 | N-[(2R)-5-Methyl-8-(4-methylpiperazin-1-yl)-1,2,3,4- | 442 |
| | tetrahydronaphthalen-2-yl]-2,1,3-benzoxadiazole-4- | |
| | sulfonamide | |
| 74 | 5-Isoxazol-3-yl-N-[(2R)-5-methyl-8-(4-methylpiperazin- | 473 |
| | 1-yl)-1,2,3,4-tetrahydronaphthalen-2-yl]thiophene-2- | |
| | sulfonamide | |
| 75 | 2-Methyl-N-[(2R)-5-methyl-8-(4-methylpiperazin-1-yl)- | 459 |
| | 1,2,3,4-tetrahydronaphthalen-2-yl]-5- | |
| | nitrobenzenesulfonamide | |
| 76 | 3-Methyl-N-[(2R)-5-methyl-8-(4-methylpiperazin-1-yl)- | 414 |
| | 1,2,3,4-tetrahydronaphthalen-2-yl]benzenesulfonamide | |
| 77 | 2-Methyl-N-[(2R)-5-methyl-8-(4-methylpiperazin-1-yl)- | 459 |
| | 1,2,3,4-tetrahydronaphthalen-2-yl]-4- | |
| | nitrobenzenesulfonamide | |
| 78 | 3-Chloro-4-fluoro-N-[(2R)-5-methyl-8-(4- | 452 |
| | methylpiperazin-1-yl)-1,2,3,4-tetrahydronaphthalen-2- | |
| | yl]benzenesulfonamide | |
| 79 | 2,4-Difluoro-N-[(2R)-5-methyl-8-(4-methylpiperazin-1- | 436 |
| | yl)-1,2,3,4-tetrahydronaphthalen-2- | |
| | yl]benzenesulfonamide | |
| 80 | 5-Fluoro-2-methyl-N-[(2R)-5-methyl-8-(4- | 432 |
| | methylpiperazin-1-yl)-1,2,3,4-tetrahydronaphthalen-2- | |
| | yl]benzenesulfonamide | |
| 81 | 1-[(1S,4R)-7,7-Dimethyl-2-oxobicyclo[2.2.1]hept-1-yl]- | 474 |
| | N-[(2R)-5-methyl-8-(4-methylpiperazin-1-yl)-1,2,3,4- | |
| | tetrahydronaphthalen-2-yl]methanesulfonamide | |
| 82 | 2-Fluoro-N-[(2R)-5-methyl-8-(4-methylpiperazin-1-yl)- | 418 |
| | 1,2,3,4-tetrahydronaphthalen-2-yl]benzenesulfonamide | |
| 83 | 3,4-Dimethoxy-N-[(2R)-5-methyl-8-(4-methylpiperazin- | 460 |
| | 1-yl)-1,2,3,4-tetrahydronaphthalen-2- | |
| | yl]benzenesulfonamide | |
| 84 | 5-Chloro-N-[(2R)-5-methyl-8-(4-methylpiperazin-1-yl)- | 440 |
| | 1,2,3,4-tetrahydronaphthalen-2-yl]thiophene-2- | |
| | sulfonamide | |
| 85 | 2-Chloro-6-methyl-N-[(2R)-5-methyl-8-(4- | 448 |
| | methylpiperazin-1-yl)-1,2,3,4-tetrahydronaphthalen-2- | |
| | yl]benzenesulfonamide | |
| 86 | 5-Bromo-N-[(2R)-5-methyl-8-(4-methylpiperazin-1-yl)- | 485 |
| | 1,2,3,4-tetrahydronaphthalen-2-yl]thiophene-2- | |
| | sulfonamide | |
| 87 | 4-Chloro-N-[(2R)-5-methyl-8-(4-methylpiperazin-1-yl)- | 479 |
| | 1,2,3,4-tetrahydronaphthalen-2-yl]-3- | |
| | nitrobenzenesulfonamide | |
| 88 | 3-Fluoro-N-[(2R)-5-methyl-8-(4-methylpiperazin-1-yl)- | 418 |
| | 1,2,3,4-tetrahydronaphthalen-2-yl]benzenesulfonamide | |
| 89 | N-[2-Chloro-4-({[(2R)-5-methyl-8-(4-methylpiperazin-1- | 491 |
| | yl)-1,2,3,4-tetrahydronaphthalen-2- | |
| | yl]amino}sulfonyl)phenyl]acetamide | |
| 90 | 2-Methoxy-5-methyl-N-[(2R)-5-methyl-8-(4- | 444 |
| | methylpiperazin-1-yl)-1,2,3,4-tetrahydronaphthalen-2- | |
| | yl]benzenesulfonamide | |
| 91 | N-[(2R)-5-Methyl-8-(4-methylpiperazin-1-yl)-1,2,3,4- | 445 |
| | tetrahydronaphthalen-2-yl]-2-nitrobenzenesulfonamide | |
| 92 | 3,4,5-Trimethoxy-N-[(2R)-5-methyl-8-(4- | 490 |
| | methylpiperazin-1-yl)-1,2,3,4-tetrahydronaphthalen-2- | |
| | yl]benzenesulfonamide | |
| 93 | 4-Bromo-N-[(2R)-5-methyl-8-(4-methylpiperazin-1-yl)- | 479 |
| | 1,2,3,4-tetrahydronaphthalen-2-yl]benzenesulfonamide | |
| 94 | 2-Bromo-N-[(2R)-5-methyl-8-(4-methylpiperazin-1-yl)- | 479 |
| | 1,2,3,4-tetrahydronaphthalen-2-yl]benzenesulfonamide | |
| 95 | N-[(2R)-5-Methyl-8-(4-methylpiperazin-1-yl)-1,2,3,4- | 468 |
| | tetrahydronaphthalen-2-yl]-4-(trifluoromethyl) | |
| | benzenesulfonamide | |
| 96 | 4-Ethyl-N-[(2R)-5-methyl-8-(4-methylpiperazin-1-yl)- | 428 |
| | 1,2,3,4-tetrahydronaphthalen-2-yl]benzenesulfonamide | |
| 97 | N-[(2R)-5-Methyl-8-(4-methylpiperazin-1-yl)-1,2,3,4- | 513 |
| | tetrahydronaphthalen-2-yl]-2-nitro-4-(trifluoromethyl) | |
| | benzenesulfonamide | |
| 98 | 4-Methyl-N-[(2R)-5-methyl-8-(4-methylpiperazin-1-yl)- | 459 |
| | 1,2,3,4-tetrahydronaphthalen-2-yl]-3-nitrobenzene | |
| | sulfonamide | |
| 99 | N-[(2R)-5-Methyl-8-(4-methylpiperazin-1-yl)-1,2,3,4- | 450 |
| | tetrahydronaphthalen-2-yl]naphthalene-1-sulfonamide | |
| 100 | N-[(2R)-5-Methyl-8-(4-methylpiperazin-1-yl)-1,2,3,4- | 469 |
| | tetrahydronaphthalen-2-yl]-2-oxo-1,2,3,4- | |
| | tetrahydroquinoline-6-sulfonamide | |
| 101 | 1-[(1R,4S)-7,7-Dimethyl-2-oxobicyclo[2.2.1]hept-1-yl]- | 474 |
| | N-[(2R)-5-methyl-8-(4-methylpiperazin-1-yl)-1,2,3,4- | |
| | tetrahydronaphthalen-2-yl]methanesulfonamide | |
| 102 | 3,4-Difluoro-N-[(2R)-5-methyl-8-(4-methylpiperazin-1- | 436 |
| | yl)-1,2,3,4-tetrahydronaphthalen-2- | |
| | yl]benzenesulfonamide | |
| 103 | 2-Methyl-N-[(2R)-5-methyl-8-(4-methylpiperazin-1-yl)- | 414 |
| | 1,2,3,4-tetrahydronaphthalen-2-yl]benzenesulfonamide | |
| 104 | 2,5-Dimethyl-N-[(2R)-5-methyl-8-(4-methylpiperazin-1- | 428 |
| | yl)-1,2,3,4-tetrahydronaphthalen-2- | |
| | yl]benzenesulfonamide | |
| 105 | N-[(2R)-5-Methyl-8-(4-methylpiperazin-1-yl)-1,2,3,4- | 493 |
| | tetrahydronaphthalen-2-yl]-4-(pyridin-3- | |
| | yloxy)benzenesulfonamide | |
| 106 | 4-Chloro-2,5-dimethoxy-N-[(2R)-5-methyl-8-(4- | 494 |
| | methylpiperazin-1-yl)-1,2,3,4-tetrahydronaphthalen-2- | |
| | yl]benzenesulfonamide | |
| 107 | 6-Chloro-N-[(2R)-5-methyl-8-(4-methylpiperazin-1-yl)- | 538 |
| | 1,2,3,4-tetrahydronaphthalen-2-yl]-2H-1,2,4- | |
| | benzothiadiazine-7-sulfonamide 1,1-dioxide | |
| 108 | Methyl 2-methyl-5-({[(2R)-5-methyl-8-(4- | 462 |
| | methylpiperazin-1-yl)-1,2,3,4-tetrahydronaphthalen-2- | |
| | yl]amino}sulfonyl)-3-furoate | |
| 109 | 2-Methoxy-5-({[(2R)-5-methyl-8-(4-methylpiperazin-1- | 473 |
| | yl)-1,2,3,4-tetrahydronaphthalen-2-yl]amino}sulfonyl) | |
| | benzamide | |
| 110 | 3-Cyano-4-fluoro-N-[(2R)-5-methyl-8-(4- | 443 |
| | methylpiperazin-1-yl)-1,2,3,4-tetrahydronaphthalen-2- | |
| | yl]benzenesulfonamide | |
| 111 | 3-Fluoro-4-methyl-N-[(2R)-5-methyl-8-(4- | 432 |
| | methylpiperazin-1-yl)-1,2,3,4-tetrahydronaphthalen-2- | |
| | yl]benzenesulfonamide | |
| 112 | 4-Fluoro-2-methyl-N-[(2R)-5-methyl-8-(4- | 432 |
| | methylpiperazin-1-yl)-1,2,3,4-tetrahydronaphthalen-2- | |
| | yl]benzenesulfonamide | |
| 113 | 2-Methoxy-N-[(2R)-5-methyl-8-(4-methylpiperazin-1- | 475 |
| | yl)-1,2,3,4-tetrahydronaphthalen-2-yl]-4- | |
| | nitrobenzenesulfonamide | |
| 114 | 2,4,5-Trifluoro-N-[(2R)-5-methyl-8-(4-methylpiperazin- | 454 |
| | 1-yl)-1,2,3,4-tetrahydronaphthalen-2- | |
| | yl]benzenesulfonamide | |
| 115 | Methyl 3-[4-({[(2R)-5-methyl-8-(4-methylpiperazin-1- | 486 |
| | yl)-1,2,3,4-tetrahydronaphthalen-2- | |
| | yl]amino}sulfonyl)phenyl] propanoate | |
| 116 | N-[(2R)-5-Methyl-8-(4-methylpiperazin-1-yl)-1,2,3,4- | 493 |
| | tetrahydronaphthalen-2-yl]-6-phenoxypyridine-3- | |
| | sulfonamide | |
| 117 | N-[(2R)-5-Methyl-8-(4-methylpiperazin-1-yl)-1,2,3,4- | 458 |
| | tetrahydronaphthalen-2-yl]-2,3-dihydro-1,4- | |
| | benzodioxine-6-sulfonamide | |
| 118 | N-[(2R)-5-Methyl-8-(4-methylpiperazin-1-yl)-1,2,3,4- | 440 |
| | tetrahydronaphthalen-2-yl]-1-benzofuran-2-sulfonamide | |
| 119 | 4-Chloro-N1-[(2R)-5-methyl-8-(4-methylpiperazin-1-yl)- | 514 |
| | 1,2,3,4-tetrahydronaphthalen-2-yl]benzene-1,3- | |
| | disulfonamide | |
| 120 | 4-Fluoro-N-[(2R)-5-methyl-8-(4-methylpiperazin-1-yl)- | 468 |
| | 1,2,3,4-tetrahydronaphthalen-2-yl]naphthalene-1- | |
| | sulfonamide | |
| 121 | 3,5-Difluoro-N-[(2R)-5-methyl-8-(4-methylpiperazin-1- | 436 |
| | yl)-1,2,3,4-tetrahydronaphthalen-2- | |
| | yl]benzenesulfonamide | |
| 122 | 4-Fluoro-N-[(2R)-5-methyl-8-(4-methylpiperazin-1-yl)- | 486 |
| | 1,2,3,4-tetrahydronaphthalen-2-yl]-3-(trifluoromethyl) | |
| | benzenesulfonamide | |
| 123 | 2-Chloro-4,5-difluoro-N-[(2R)-5-methyl-8-(4- | 470 |
| | methylpiperazin-1-yl)-1,2,3,4-tetrahydronaphthalen-2- | |
| | yl]benzenesulfonamide | |
| 124 | 5-Chloro-2,4-difluoro-N-[(2R)-5-methyl-8-(4- | 470 |
| | methylpiperazin-1-yl)-1,2,3,4-tetrahydronaphthalen-2- | |
| | yl]benzenesulfonamide | |
| 125 | 4-Chloro-2,5-difluoro-N-[(2R)-5-methyl-8-(4- | 470 |
| | methylpiperazin-1-yl)-1,2,3,4-tetrahydronaphthalen-2- | |
| | yl]benzenesulfonamide | |
| 126 | 3-Chloro-4-methyl-N-[(2R)-5-methyl-8-(4- | 448 |
| | methylpiperazin-1-yl)-1,2,3,4-tetrahydronaphthalen-2- | |
| | yl]benzenesulfonamide | |
| 127 | N-[(2R)-5-Methyl-8-(4-methylpiperazin-1-yl)-1,2,3,4- | 478 |
| | tetrahydronaphthalen-2-yl]-2- | |
| | (methylsulfonyl)benzenesulfonamide | |
| 128 | 1,3,5-Trimethyl-N-[(2R)-5-methyl-8-(4-methylpiperazin- | 432 |
| | 1-yl)-1,2,3,4-tetrahydronaphthalen-2-yl]-1H-pyrazole-4- | |
| | sulfonamide | |
| 129 | N-[(2R)-5-Methyl-8-(4-methylpiperazin-1-yl)-1,2,3,4- | 459 |
| | tetrahydronaphthalen-2-yl]-1-(3- | |
| | nitrophenyl)methanesulfonamide | |
| 130 | 5-Methyl-N-[(2R)-5-methyl-8-(4-methylpiperazin-1-yl)- | 472 |
| | 1,2,3,4-tetrahydronaphthalen-2-yl]-2,1,3- | |
| | benzothiadiazole-4-sulfonamide | |
| 131 | 2,5-Dimethoxy-N-[(2R)-5-methyl-8-(4-methylpiperazin- | 505 |
| | 1-yl)-1,2,3,4-tetrahydronaphthalen-2-yl]-4- | |
| | nitrobenzenesulfonamide | |
| 132 | N-[(2R)-5-Methyl-8-(4-methylpiperazin-1-yl)-1,2,3,4- | 467 |
| | tetrahydronaphthalen-2-yl]-1-oxo-1,2- | |
| | dihydroisoquinoline-4-sulfonamide | |
| 133 | Dimethyl 5-({[(2R)-5-methyl-8-(4-methylpiperazin-1- | 516 |
| | yl)-1,2,3,4-tetrahydronaphthalen-2- | |
| | yl]amino}sulfonyl)isophthalate | |
| 134 | 4-Methyl-N-[(2R)-5-methyl-8-(4-methylpiperazin-1-yl)- | 414 |
| | 1,2,3,4-tetrahydronaphthalen-2-yl]benzenesulfonamide | |
| 135 | 4-Methoxy-N-[(2R)-5-methyl-8-(4-methylpiperazin-1- | 430 |
| | yl)-1,2,3,4-tetrahydronaphthalen-2- | |
| | yl]benzenesulfonamide | |
| 136 | 2-Chloro-N-[(2R)-5-methyl-8-(4-methylpiperazin-1-yl)- | 434 |
| | 1,2,3,4-tetrahydronaphthalen-2-yl]benzenesulfonamide | |
| 137 | N-[(2R)-5-Methyl-8-(4-methylpiperazin-1-yl)-1,2,3,4- | 468 |
| | tetrahydronaphthalen-2-yl]-3- | |
| | (trifluoromethyl)benzenesulfonamide | |
| 138 | N-[(2R)-5-Methyl-8-(4-methylpiperazin-1-yl)-1,2,3,4- | 483 |
| | tetrahydronaphthalen-2-yl]-5-pyridin-2-ylthiophene-2- | |
| | sulfonamide | |
| 139 | 2-Cyano-N-[(2S)-5-methyl-8-(4-methylpiperazin-1-yl)- | 425 |
| | 1,2,3,4-tetrahydronaphthalen-2-yl]benzenesulfonamide | |
| 140 | 4-Bromo-2-fluoro-N-[(2R)-5-methyl-8-(4- | 497 |
| | methylpiperazin-1-yl)-1,2,3,4-tetrahydronaphthalen-2- | |
| | yl]benzenesulfonamide | |
| 141 | N-[3-({[(2R)-5-Methyl-8-(4-methylpiperazin-1-yl)- | 457 |
| | 1,2,3,4-tetrahydronaphthalen-2- | |
| | yl]amino}sulfonyl)phenyl]acetamide | |
| 142 | 3-Chloro-2-methyl-N-[(2R)-5-methyl-8-(4- | 448 |
| | methylpiperazin-1-yl)-1,2,3,4-tetrahydronaphthalen-2- | |
| | yl]benzenesulfonamide | |
| 143 | 4-Methoxy-N-[(2R)-5-methyl-8-(4-methylpiperazin-1- | 475 |
| | yl)-1,2,3,4-tetrahydronaphthalen-2-yl]-2-nitrobenzene | |
| | sulfonamide | |
| 144 | 3-Methoxy-N-[(2R)-5-methyl-8-(4-methylpiperazin-1- | 430 |
| | yl)-1,2,3,4-tetrahydronaphthalen-2- | |
| | yl]benzenesulfonamide | |
| 145 | N-[(2R)-5-Methyl-8-(4-methylpiperazin-1-yl)-1,2,3,4- | 540 |
| | tetrahydronaphthalen-2-yl]-2- | |
| | (phenylsulfonyl)benzenesulfonamide | |
| 146 | (E)—N-[(2R)-5-Methyl-8-(4-methylpiperazin-1-yl)- | 426 |
| | 1,2,3,4-tetrahydronaphthalen-2-yl]-2- | |
| | phenylethylenesulfonamide | |
| 147 | 2-Methoxy-4-methyl-N-[(2R)-5-methyl-8-(4- | 444 |
| | methylpiperazin-1-yl)-1,2,3,4-tetrahydronaphthalen-2- | |
| | yl]benzenesulfonamide | |
| 148 | 3-(Difluoromethoxy)-N-[(2R)-5-methyl-8-(4- | 466 |
| | methylpiperazin-1-yl)-1,2,3,4-tetrahydronaphthalen-2- | |
| | yl]benzenesulfonamide | |
| 149 | 4-Bromo-3-fluoro-N-[(2R)-5-methyl-8-(4- | 497 |
| | methylpiperazin-1-yl)-1,2,3,4-tetrahydronaphthalen-2- | |
| | yl]benzenesulfonamide | |
| 150 | N-[(2R)-5-Methyl-8-(4-methylpiperazin-1-yl)-1,2,3,4- | 490 |
| | tetrahydronaphthalen-2-yl]-5-(1,2,3-thiadiazol-4- | |
| | yl)thiophene-2-sulfonamide | |
| 151 | Methyl 2,5-dimethyl-4-({[(2S)-5-methyl-8-(4- | 476 |
| | methylpiperazin-1-yl)-1,2,3,4-tetrahydronaphthalen-2- | |
| | yl]amino}sulfonyl)-3-furoate | |

### Intermediate 1

### (2R)-5-Bromo-8-methoxy-1,2,3,4-tetrahydronaphthalen-2-amine

(2R)-8-Methoxy-1,2,3,4-tetrahydronaphthalen-2-amine hydrochloride (10 g, 47 mmol) and sodium acetate (11.5 g, 140 mmol) were stirred in acetic acid (450 ml) under nitrogen atmosphere for 30 min. Bromine (2.52 ml, 49 mmol) was added and the mixture was stirred at ambient temperature for 10 min. The mixture was concentrated and EtOAC (700 ml) and aqueous ammonia was added. A solid formed in the aqueous phase. The mixture was stirred for 15 min and EtOAc (100 ml) and water (100 ml) were added. The mixture was shaken and the organic phase was decanted leaving the solids in the aqueous phase. The aqueous phase was diluted with water and the slurry was extracted with ether. The ether phase was dried (MgSO₄) and the solvent was evaporated to give a solid (3.1 g). The pH in the remaining aqueous phase was adjusted to 12 by addition of 45% aqueous sodium hydroxide. The solid formed was isolated by filtration (3.8 g). The solid from the ether extraction was combined with the solid isolated by filtration to give the title compound (6.9 g, 58%). EI-MS (70 eV) m/z M⁺ 255, 257.

### Intermediate 2

### (2R)—N,N-Dibenzyl-5-bromo-8-methoxy-1,2,3,4-tetrahydronaphthalen-2-amine

(2R)-5-Bromo-8-methoxy-1,2,3,4-tetrahydronaphthalen-2-amine (7.0 g, 27 mmol) potassium iodide (100 mg, 0.60 mmol) and potassium carbonate (15 g, 108 mmol) were mixed in anhydrous acetonitrile (400 ml) under nitrogen atmosphere. Benzylbromide (7.1 ml, 60 mmol) was added and the mixture was heated at reflux for 14 h. The mixture was filtered and the filtrate was concentrated by evaporation. Diethyl ether was added and the mixture was washed with aqueous ammonia (2M) and brine. The organic phase was dried (MgSO₄) and the solvent was evaporated. The residue was purified by column chromatography on silica eluting with dichloromethane:hexane 1:4 to give an oil that solidified upon storage (10 g, 85%). m.p. 100-101° C.; EI-MS (70 eV) m/z M⁺ 437, 439.

### Intermediate 3

### (2R)—N,N-Dibenzyl-8-methoxy-5-(4-methylpiperazin-1-yl)-1,2,3,4-tetrahydronaphthalen-2-amine

(2R)—N,N-Dibenzyl-5-bromo-8-methoxy-1,2,3,4-tetrahydronaphthalen-2-amine (570 mg, 1.3 mmol) was dissolved in toluene (13 ml). The mixture was put under nitrogen atmosphere and N-methylpiperazine (0.16 ml, 1.4 mmol) was added followed by BINAP (97 mg, 0.16 mmol) Pd₂(dba)₃ (71 mg, 0.08 mmol) and sodium t-butoxide (174 mg, 1.8 mmol). The mixture was heated at 85° C. under nitrogen atmosphere for 20 h. The mixture was filtered and the filtrate was concentrated. The residue was purified by chromatography on silica eluting with gradients of ammonia in methanol and chloroform to give a solid (0.39 g, 66%). ESI-MS m/z M+H⁺ 456.

### Intermediate 4

### (2R)-8-Methoxy-5-(4-methylpiperazin-1-yl)-1,2,3,4-tetrahydronaphthalen-2-amine

(2R)—N,N-Dibenzyl-8-methoxy-5-(4-methylpiperazin-1-yl)-1,2,3,4-tetrahydronaphthalen-2-amine (392 mg, 0.86 mmol) was dissolved in methanol (20 ml) and ammonium formate (1.6 g, 25 mmol) was added. The mixture was put under nitrogen atmosphere and palladium on charcoal (10%, 100 mg) was added. The mixture was heated at 50° C. for 6 h. The mixture was filtered and the solids were washed with methanol. The filtrate was concentrated, dichloromethane was added and the mixture was washed with aqueous sodium hydroxide (2M) and brine. The organic phase was dried Na₂SO₄) and the solvent was evaporated to give a solid (170 mg, 71%). ESI-MS m/z M+H⁺ 276.

### Intermediate 5

### 2,2,2-Trifluoro-N-[(2S)-8-methoxy-1,2,3,4-tetrahydronaphthalen-2-yl]acetamide

(2R)-8-Methoxy-1,2,3,4-tetrahydronaphthalen-2-amine hydrochloride (23.3 g, 109 mmol) was suspended in dichloromethane (300 ml). DIPEA (44.5 ml, 255 mmol) was added and the reaction flask was placed in a room tempered waterbath. Trifluoroacetic arhydride (17.3 ml, 124 mmol) was added over 10 min and the reaction mixture was stirred for 2 h at ambient temperature. The mixture was washed with saturated aqueous sodium hydrogen carbonate (×3) dried (Na₂SO₄) and the solvent was evaporated to give a solid (21 g, 77%). ESI-MS m/z M+H⁺ 274.

### Intermediate 6

### N-[(2S)-5-Bromo-8-methoxy-1,2,3,4-tetrahydronaphthalen-2-yl]-2,2,2-trifluoroacetamide

2,2,2-Trifluoro-N-[(2S)-8-methoxy-1,2,3,4-tetrahydronaphthalen-2-yl]acetamide (8.0 g, 29 mmol) and sodium acetate (7.4 g, 90 mmol) were dissolved in acetic acid (120 ml). Bromine (1.6 ml, 31 mmol) was dissolved in acetic acid (40 ml) and was added to the reaction mixture over 2 b. The mixture was stirred for 1 h and then poured onto ice. The solid formed was isolated by filtration and washed with water. The solid was dissolved in dichloromethane and was washed with saturated aqueous sodium hydrogen carbonate, dried (Na₂SO₄) filtered and the solvent was removed to give a solid (8.8 g, 86%). ESI-MS m/z M−H⁺ 350, 352.

### Intermediate 7

### tert-Butyl 4-{(6S)-4-methoxy-6-[(trifluoroacetyl)amino]-5,6,7,8-tetrahydronaphthalen-1-yl}piperazine-1-carboxylate

N-[(2S)-5-Bromo-8-methoxy-1,2,3,4-tetrahydronaphthalen-2-yl]-2,2,2-trifluoroacetamide (5.0 g, 14.2 mmol) 4-Boc-piperazine (3.2 g, 17.2 mmol) Pd₂(dba)₃ (337 mg, 0.37 mmol) bis(2-diphenylphosphinophenyl)ether (398 mg, 0.74 mmol) and sodium t-butoxide (4.09 g, 42.6 mmol) were suspended in toluene (90 ml). The mixture was heated at 100° C. under argon atmosphere for 2 h. EtOAc was added and the mixture was washed with saturated aqueous sodium hydrogen carbonate (×2) dried, filtered and the solvent was evaporated. The residue was purified by column chromatography on silica eluting with gradients of EtOAc and heptane to give a solid (0.5 g, 7%). ESI-MS D/z M+H⁺ 458.

### Intermediate 8

### tert-Butyl 4-[(6S)-6-amino-4-methoxy-5,6,7,8-tetrahydronaphthalen-1-yl]piperazine-1-carboxylate

tert-Butyl 4-{(6S)-4-methoxy-6-[(trifluoroacetyl)amino]-5,6,7,8-tetrahydronaphthalen-1-yl}piperazine-1-carboxylate (490 mg) was dissolved in methanol (20 ml). Aqueous sodium hydroxide (2.5 M, 5 ml) was added and the mixture was stirred at ambient temperature for 4 days. The mixture was concentrated and dichloromethane was added. The organic layer was washed with aqueous sodium hydrogen carbonate, dried Na₂SO₄) filtered and the solvent was evaporated to give the title compound (0.35 g). ESI-MS m/z M+H⁺ 362.

### Pharmacology

Method for [125I]SB258585 binding to rat striatal 5-HT6 receptors

### Materials

[¹²⁵I]SB258585 (1) with specific radioactivity 2000 Ci/mmol was purchased from Amersham Biosciences Europe GmbH, Freiburg, Germany. Other chemicals were purchased from commercial sources and were of analytical grade.

### Preparation of Membranes

Striatal tissue from adult rats (Sprague-Dawley, 320-370 g, B & K Sweden) were dissected out, weighed and homogenized in buffer containing 50 mM Tris-HCl, 4 mM MgCl₂, 1 mM EDTA, 10 μM pargyline and protease inhibitor (Complete, Roche Diagnostics) pH 7.4 using an Ultra-Turrax T8 (IKA Labortechnik, Germany). The tissue homogenate was centrifuged at 48 000×g for 10 min and the pellet was resuspended and recentrifuged as above. The final membranes were diluted in buffer to a concentration of 60 mg original wet weight (w.w.) per ml and stored in aliquots at −70° C.

### Radioligand Binding Assays

Saturation binding studies were carried out in duplicate with 1-3 mg w.w. per tube in 0.5 ml buffer (50 mM Tris, 4 mM MgCl₂, 100 mM NaCl, 1 mM EDTA, 5 mM ascorbate and 10 μM pargyline at pH 7.4), 0.2 nM [¹²⁵I]SB258585 and unlabelled SB258585 to give a final concentration range of 0.23-20 nM (12 conc.). Non-specific binding was determined in the presence of 10 μM methiothepin. In the competition experiments 0.8-2 mg w.w. per tube and a radioligand concentration of 0.5-1 nM were used with 7 concentrations of the competing drug pre-dissolved in DMSO and diluted in buffer. The assays were incubated for 1-3 hours at room temperature, and terminated by rapid filtration through Whatman GF/B filters pretreated with 0.3% polyethyleneimine using a Brandel cell harvester. The radioactivity was determined in a Packard Tri-Carb 2900TR liquid scintillation counter. Data were analyzed by non-linear regression analyses using PRISM 4.00 (GraphPad Software Inc., San Diego, Calif.).

More information about the assay can be found in Hirst, W. D., Minton, J. A. L., Bromidge, S. M., Moss, S. F., Latter, A., Riley, G., Routledge, C., Middlemiss, D. N. & Price, G. W. (2000). Characterization of [¹²⁵I]-SB-258585 binding to human recombinant and native 5-HT6 receptors in rat, pig and human brain tissue is described in Br. J. Pharmacol., 130, 1597-1605.

### Results

Typical IC₅₀ values as measured in the assays described above are 1 μM or less. In one aspect of the invention the IC₅₀ is below 500 nM. In another aspect of the invention the IC₅₀ is below 50 nM. In a further aspect of the invention the IC₅₀ is below 10 nM.

Specimen results from assay.

| Example no | Kᵢ (nM) |
|---|---|
| 12 | 0.8 |

1-18. (canceled)

19. A compound having the formula I, wherein: 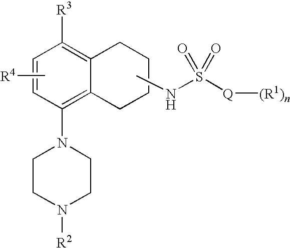
Q is selected from C₆₋₁₀aryl₀₋₆alkyl, C₅₋₁₁heteroarylC₀₋₆alkyl, C₃₋₈cycloalkylC₀₋₆alkyl, C₃₋₈heterocycloalkylC₀₋₆alkyl, C₂₋₆alkenyl and C₂₋₁₀alkyl;
R₁ is selected from hydrogen, hydroxy, halogen, C₁₋₁₀alkyl, C₂₋₁₀alkenyl, C₂₋₁₀alkynyl, C₁₋₁₀alkoxy, N(R⁵)₂, C₆₋₁₀arylC₀₋₆alkyl, C₅₋₁₁heteroarylC₀₋₆alkyl, C₁₋₆haloalkyl, R⁵OC₀₋₆alkyl, cyano, SR⁵, R⁶SO₂C₀₋₆alkyl, SOR⁶, R⁵CON(R⁵)C₀₋₆alkyl, N(R⁵)SO₂R⁵, COR⁶, R⁵CO₂C₀₋₆alkyl, R⁵OC(O)C₀₋₆alkyl, OSO₂R⁵, C₃₋₈cycloalkyl, C₃₋₈heterocycloalkyl, (R⁵)₂NCOC₀₋₆alkyl, SO₂N(R⁵)₂, N(R⁵)CON(R⁵)₂, N(R⁷)COR⁸, NO₂, OR⁵ and oxo;
n is 0, 1, 2, 3 or 4;
R² is selected from hydrogen, C₁₋₆alkyl, R⁷OC₂₋₆alkyl, C₁₋₆haloalkyl, cyanoC₁₋₆alkyl, (R⁷)₂NCOC₁₋₆alkyl and R⁷CON(R⁷)C₁₋₆alkyl;
R³ is selected from hydrogen, C₁₋₆alkyl, halogen, cyano, C₁₋₆alkoxy, R⁷OC₀₋₆alkyl, C₁₋₆haloalkyl, cyanoC₁₋₆alkyl, NO₂, (R⁷)₂NCOC₀₋₃alkyl or R⁷CON(R⁷)C₀₋₃alkyl;
R⁴ is seleceted from hydrogen, C₁₋₆alkyl, halogen, cyano, C₁₋₆alkoxy, R⁷OC₀₋₆alkyl, C₁₋₆haloalkyl, cyanoC₁₋₆alkyl, NO₂, (R⁷)₂NCOC₀₋₃alkyl and R⁷CON(R⁷)C₀₋₃alkyl;
R⁵ is selected from hydrogen, C₁₋₁₀alkyl, C₁₋₆haloalkyl, C₃₋₈cycloalkylC₀₋₆alkyl, C₃₋₈heterocycloalkylC₀₋₆alkyl, C₆₋₁₀arylC₀₋₆alkyl and C₅₋₆heteroarylC₅₋₆alkyl;
R⁶ is selected from C₁₋₁₀alkyl, C₁₋₆haloalkyl, C₁₋₆alkoxy, C₃₋₈cycloalkylC₀₋₃alkyl, C₃₋₈heterocycloalkylC₀₋₆alkyl, C₆₋₁₀arylC₀₋₃alkyl and C₅₋₆heteroarylC₀₋₃alkyl; and
wherein any aryl, cycloalkyl, heterocycloalkyl or heteroaryl as defined for R¹, R⁵ and R⁶ may be substituted by one or more groups independently selected from hydrogen, halogen, hydroxy, C₁₋₃haloalkyl, cyano, OR⁸, C₁₋₆alkyl, oxo, CON(R⁷)₂, N(R⁷)COR⁸, SO₂R⁸, SOR⁸, N(R⁷)₂ and COR⁷;
R⁷ is hydrogen, C₁₋₆alkyl or C₁₋₆haloalkyl; and
R⁸ is C₁₋₆alkyl or C₁₋₆haloalkyl;
or pharmaceutically acceptable salts, solvates or solvated salts thereof.

20. The compound according to claim 19, wherein:
Q is selected from C₆₋₁₀arylC₀₋₆alkyl, C₅₋₁₁heteroarylC₀₋₆alkyl, C₃₋₈cycloalkylC₀₋₆alkyl, C₃₋₈heterocycloalkylC₀₋₆alkyl and C₂₋₁₀alkyl;
R¹ is selected from hydrogen, halogen, C₁₋₁₀alkyl, C₁₋₁₀alkoxy, N(R⁵)₂, C₆₋₁₀arylC₀₋₆alkyl, C₅₋₁₁heteroarylC₀₋₆alkyl, C₁₋₆haloalkyl, R⁵OC₀₋₆alkyl, cyano, SR⁵, R⁶SO₂C₀₋₆alkyl, N(R⁵)SO₂R⁵, COR⁶, R⁵CO₂C₀₋₆alkyl, R⁵OC(O)C₀₋₆alkyl, OSO₂R⁵, C₃₋₈cycloalkyl, C₃₋₈heterocycloalkyl,
(R⁵)₂NCOC₀₋₆alkyl, SO₂N(R⁵)₂, N(R⁵)CON(R⁵)₂, N(R⁷)COR⁸, OR⁵, NO₂ and oxo;
n is 0, 1, 2, 3 or 4;
R² is selected from hydrogen, C₁₋₆alkyl, R⁷OC₂₋₆alkyl and C_{1- 6}haloalkyl;
R³ is selected from hydrogen, C₁₋₆alkyl, halogen cyano and C₁₋₆alkoxy;
R⁴ is selected from hydrogen, C₁₋₆alkyl, halogen, cyano, C₁₋₆alkoxy, R⁷OC₀₋₆alkyl, C₁₋₆haloalkyl, cyanoC₁₋₆alkyl, NO₂, (R⁷)₂NCOC₀₋₃alkyl and R⁷CON(R⁷)C₀₋₃alkyl;
R⁵ is selected from hydrogen, C₁₋₁₀alkyl, C₁₋₆haloalkyl, C₃₋₈cycloalkylC₀₋₆alkyl, C₃₋₈heterocycloalkylC₀₋₆alkyl and C₆₋₁₀arylC₀₋₆alkyl;
R⁶ is selected from C₁₋₁₀alkyl, C₁₋₆haloalkyl, C₁₋₆alkoxy, C₃₋₈cycloalkylC₀₋₃alkyl and C₃₋₈heterocycloalkylC₀₋₆alkyl; and
wherein any aryl, cycloalkyl, heterocycloalkyl or heteroaryl as defined for R¹, R⁵ and R⁶ may be substituted by one or more groups independently selected from hydrogen, halogen, hydroxy, C₁₋₃haloalkyl, cyano, OR⁸, C₁₋₆alkyl, oxo, CON(R⁷)₂, N(R⁷)COR⁸, SO₂R⁸, SOR⁸, N(R⁷)₂ and COR⁷;
R⁷ is hydrogen, C₁₋₆alkyl or C₁₋₆haloalkyl; and
R⁸ is C₁₋₆alkyl or C₁₋₆haloalkyl;
or pharmaceutically acceptable salts, solvates or solvated salts thereof.

21. The compound according to claim 19, wherein:
Q is selected from C₆₋₁₀arylC₀₋₆alkyl, C₅₋₁₁heteroarylC₀₋₆alkyl, C₃₋₈cycloalkylC₀₋₆alkyl and C₃₋₈heterocycloalkylC₀₋₆alkyl;
R¹ is selected from hydrogen, halogen, C₁₋₁₀alkyl, C₁₋₁₀alkoxy, N(R⁵)₂, C₆₋₁₀arylC₀₋₆alkyl, C₅₋₁₁heteroarylC₀₋₆alkyl, C₁₋₆haloalkyl, R⁵OC₀₋₆alkyl, cyano, SR⁵, R⁶SO₂C₀₋₆alkyl, N(R⁵)SO₂R⁵, COR⁶, R⁵CO₂C₀₋₆alkyl, R⁵OC(O)C₀₋₆alkyl, OSO₂R⁵, C₃₋₈cycloalkyl, C₃₋₈heterocycloalkyl, (R⁵)₂NCOC₀₋₆alkyl, SO₂N(R⁵)₂, N(R⁵)CON(R⁵)₂, N(R⁷)COR⁸, OR⁵, NO₂ and oxo;
n is 0, 1, 2, 3 or 4;
R² is selected from hydrogen, C₁₋₆alkyl and C₁₋₆haloalkyl;
R³ is selected from hydrogen, C₁₋₆alkyl, halogen and C₁₋₆alkoxy;
R⁴ is selected from hydrogen, C₁₋₆alkyl, halogen, cyano and C₁₋₆alkoxy;
R⁵ is selected from hydrogen, C₁₋₁₀alkyl, C₃₋₈cycloalkylC₀₋₆alkyl, C₃₋₈heterocycloalkylC₀₋₆alkyl and C₆₋₁₀arylC₀₋₆alkyl;
R⁶ is selected from C₁₋₁₀alkyl, C₁₋₆haloalkyl and C₁₋₆alkoxy; and
wherein any aryl, cycloalkyl, heterocycloalkyl or heteroaryl as defined for R¹, R⁵ and R⁶ may be substituted by one or more groups independently selected from hydrogen, halogen, hydroxy, C₁₋₃haloalkyl, cyano, OR⁸, C₁₋₆alkyl, oxo, CON(R⁷)₂, N(R⁷)COR⁸, SO₂R⁸, SOR⁸, N(R⁷)₂ and COR⁷;
R⁷ is hydrogen; and
R⁸ is C₁₋₆alkyl;
or pharmaceutically acceptable salts, solvates or solvated salts thereof.

22. The compound according to claim 19, wherein:
Q is selected from C₆₋₁₀arylC₀₋₆alkyl, C₅₋₁₁heteroarylC₀₋₆alkyl, C₃₋₈cycloalkylC₀₋₆alkyl and C₃₋₈heterocycloalkylC₀₋₆alkyl;
R¹ is selected from hydrogen, halogen, C₁₋₁₀alkyl, C₁₋₁₀alkoxy, C₅₋₁₁heteroarylC₀₋₆alkyl, C₁₋₆haloalkyl, R^{5OC}₀₋₆alkyl, cyano, SR⁵, R⁶SO₂C₀₋₆alkyl, COR⁶, R⁵CO₂C₀₋₆alkyl, R⁵OC(O)C₀₋₆alkyl, SO₂N(R⁵)₂, N(R⁷)COR⁸, OR⁵, NO₂ and oxo;
n is 0, 1, 2 or 3;
R² is hydrogen or C₁₋₆alkyl;
R³ is hydrogen, C₁₋₆alkyl or C₁₋₆alkoxy;
R⁴ is hydrogen, C₁₋₆alkyl or C₁₋₆alkoxy;
R⁵ is selected from hydrogen, C₁₋₁₀alkyl and C₆₋₁₀arylC₀₋₆alkyl;
R⁶ is selected from C₁₋₁₀alkyl, C₁₋₆haloalkyl and C₁₋₆alkoxy; and
wherein any aryl, cycloalkyl, heterocycloalkyl or heteroaryl as defined for R¹, R⁵ and R⁶ may be substituted by one or more groups independently selected from hydrogen, halogen, hydroxy, C₁₋₃haloalkyl, cyano, OR⁸, C₁₋₆alkyl, oxo, CON(R⁷)₂, N(R⁷)COR⁸, SO₂R⁸, SOR⁸, N(R⁷)₂ and COR⁷;
R⁷ is hydrogen; and
R⁸ is C₁₋₆alkyl;
or pharmaceutically acceptable salts, solvates or solvated salts thereof.

23. A compound according to any one of claim 19, wherein Q is phenyl.

24. A compound according to any one of claim 19, wherein
R² is hydrogen or C₁₋₃alkyl;
R³ is hydrogen, C₁₋₃alkyl or C₁₋₃alkoxy; and
R⁴ is hydrogen, C₁₋₃alkyl or C₁₋₃alkoxy.

25. A compound according to claim 19, wherein:
Q is selected from C₆₋₁₀arylC₀₋₃alkyl, C₅₋₁₁heteroarylC₀₋₃alkyl, C₃₋₈cycloalkylC₀₋₃alkyl or C₂₋₄alkenyl and C₂₋₅alkyl;
R¹ is selected from hydrogen, halogen, C₁₋₄alkyl, C₁₋₄alkoxy, C₅₋₁₁heteroarylC₀₋₃alkyl, C₁₋₆haloalkyl, R⁵OC₀₋₃alkyl, cyano, R⁶SO₂C₀₋₃alkyl, R⁵CON(R⁵)C₀₋₃alkyl, R⁵OC(O)C₀₋₆alkyl, (R⁵)₂NCOC₀₋₃alkyl, SO₂N(R⁵)₂, NO₂ and oxo;
n is 0, 1, 2, 3 or 4;
R² is hydrogen or C₁₋₃alkyl;
R³ is hydrogen, C₁₋₃alkyl or C₁₋₃alkoxy;
R⁴ is hydrogen;
R⁵ is hydrogen, C₁₋₃alkyl, C₁₋₃haloalkyl, C₆₋₁₀arylC₀₋₃alkyl or C₅₋₆heteroarylC₀₋₃alkyl;
R⁶ is C₁₋₄alkyl or C₆₋₁₀arylC₀₋₃alkyl; and
wherein any aryl, cycloalkyl, heterocycloalkyl or heteroaryl as defined for R¹, R⁵ and R⁶ may be substituted by one or more groups independently selected from hydrogen, halogen, and C₁₋₃haloalkyl;
or pharmaceutically acceptable salts, solvates or solvated salts thereof.

26. A compound according to claim 19, wherein n is 0, 1, 2 or 3.

27. Compounds selected from the group consisting of
3-chloro-N-[(2R)-8-methoxy-5-(4-methylpiperazin-1-yl)-1,2,3,4-tetrahydronaphthalen-2- yl]benzenesulfonamide,
4-chloro-N-[(2R)-8-methoxy-5-(4-methylpiperazin-1-yl)-1,2,3,4-tetrahydronaphthalen-2- yl]benzenesulfonamide,
3-chloro-N-[(2S)-5-(4-methylpiperazin-1-yl)-1,2,3,4-tetrahydronaphthalen-2- yl]benzenesulfonamide,
3-chloro-4-methyl-N-[(2S)-5-(4-methylpiperazin-1-yl)-1,2,3,4-tetrahydronaphthalen-2- yl]benzenesulfonamide,
5-chloro-N-[(2S)-5-(4-methylpiperazin-1-yl)-1,2,3,4-tetrahydronaphthalen-2-yl]naphthalene- 2-sulfonamide,
3-chloro-4-methyl-N-[(2S)-5-piperazin-1-yl- 1,2,3,4-tetrahydronaphthalen-2- yl]benzenesulfonamide,
3-chloro-N-[(2S)-5-piperazin-1-yl-1,2,3,4-tetrahydronaphthalen-2-yl]benzenesulfonamide,
3-chloro-N-[(2S)-8-methoxy-5-piperazin-1-yl-1,2,3,4-tetrahydronaphthalen-2- yl]benzenesulfonamide,
N-[(2S)-8-methoxy-5-piperazin-1-yl-1,2,3,4,-tetrahydronaphthalen-2-yl]-3,5- dimethylisoxazole-4-sulfonamide,
1-[3-chloro-5-(trifluoromethyl)pyridin-2-yl]-N-[(2S)-8-methoxy-5-piperazin-1-yl-1,2,3,4- tetrahydronaphthalen-2-yl]-1H-pyrrole-2-sulfonamide,
2,3-dichloro-N-[(2R)-5-methyl-8-(4-methylpiperazin-1-yl)-1,2,3,4-tetrahydronaphthalen-2- yl]benzenesulfonamide,
N-[(2R)-8-(4-methylpiperazin-1-yl)-1,2,3,4-tetrahydronaphthalen-2-yl]naphthalene-2- sulfonamide,
3-chloro-4-methyl-N-[(2R)-8-(4-methylpiperazin-1-yl)-1,2,3,4-tetrahydronaphthalen-2- yl]benzenesulfonamide,
N-[(2R)-5-methyl-8-(4-methylpiperazin-1-yl)-1,2,3,4-tetrahydronaphthalen-2-yl]naphthalene- 2-sulfonamide,
1-[3-chloro-5-(trifluoromethyl)pyridin-2-yl]-N-[(2R)-5-methyl-8-(4-methylpiperazin-1-yl)- 1,2,3,4-tetrahydronaphthalen-2-yl]-1H-pyrrole-2-sulfonamide,
5-chloro-N-[(2R)-5-methyl-8-piperazin-1-yl-1,2,3,4-tetrahydronaphthalen-2-yl]naphthalene-2- sulfonamide,
N-[(2R)-5-methyl-8-piperazin-1-yl-1,2,3,4-tetrahydronaphthalen-2-yl]naphthalene-2- sulfonamide,
2,6-dichloro-N-[(2R)-5-methyl-8-piperazin-1-yl-1,2,3,4-tetrahydronaphthalen-2- yl]benzenesulfonamide,
3,5-dimethyl-N-[(2R)-5-methyl-8-piperazin-1-yl-1,2,3,4-tetrahydronaphthalen-2-yl]isoxazole- 4-sulfonamide,
2-chloro-6-methyl-N-[(2R)-5-methyl-8-piperazin-1-yl-1,2,3,4-tetrahydronaphthalen-2- yl]benzenesulfonamide,
2,6-difluoro-N-[(2R)-5-methyl-8-piperazin-1-yl-1,2,3,4-tetrahydronaphthalen-2- yl]benzenesulfonamide,
N-[(2R)-5-methyl-8-piperazin-1-yl-1,2,3,4-tetrahydronaphthalen-2-yl]isoquinoline-5- sulfonamide,
5-chloro-1,3-dimethyl-N-[(2R)-5-methyl-8-piperazin-1-yl-1,2,3,4-tetrahydronaphthalen-2-yl]- 1H-pyrazole-4-sulfonamide,
2,4-dimethyl-N-[(2R)-5-methyl-8-piperazin-1-yl-1,2,3,4-tetrahydronaphthalen-2-yl]-1,3- thiazole-5-sulfonamide,
1,3,5-trimethyl-N-[(2R)-5-methyl-8-piperazin-1-yl-1,2,3,4-tetrahydronaphthalen-2-yl]-1H- pyrazole-4-sulfonamide,
4-bromo-2,5-dichloro-N-[(2R)-5-methyl-8-piperazin-1-yl-1,2,3,4-tetrahydronaphthalen-2- yl]thiophene-3-sulfonamide,
4-bromo-N-[(2R)-5-methyl-8-piperazin-1-yl-1,2,3,4-tetrahydronaphthalen-2-yl]thiophene-3- sulfonamide,
N-[4-methyl-5-({[(2R)-5-methyl-8-piperazin-1-yl- 1,2,3,4-tetrahydronaphthalen-2- yl]amino}sulfonyl)-1,3-thiazol-2-yl]acetamide,
2,4-dDimethyl-N-[(2R)-5-methyl-8-(4-methylpiperazin-1-yl)-1,2,3,4-tetrahydronaphthalen-2- yl]-1,3-thiazole-5-sulfonamide,
N-[(2R)-5-methyl-8-(4-methylpiperazin-1-yl)-1,2,3,4-tetrahydronaphthalen-2-yl]-1,3- benzothiazole-6-sulfonamide,
5-chloro-N-[(2R)-8-piperazin-1-yl-1,2,3,4-tetrahydronaphthalen-2-yl]naphthalene-2- sulfonamide,
4-chloro-N-[(2R)-8-piperazin-1-yl-1,2,3,4-tetrahydronaphthalen-2-yl]naphthalene-1- sulfonamide,
5-chloro-N-[(2R)-8-piperazin-1-yl-1,2,3,4-tetrahydronaphthalen-2-yl]naphthalene-1- sulfonamide,
1-(3-chlorophenyl)-N-[(2R)-8-piperazin-1-yl-1,2,3,4-tetrahydronaphthalen-2- yl]methanesulfonamide,
2-(4-chlorophenyl)-N-[(2R)-8-piperazin-1-yl-1,2,3,4-tetrahydronaphthalen-2- yl]ethanesulfonamide,
N-[(2R)-5-methyl-8-(4-methylpiperazin-1-yl)-1,2,3,4-tetrahydronaphthalen-2- yl]benzenesulfonamide,
3-cyano-N-[(2R)-5-methyl-8-(4-methylpiperazin-1-yl)-1,2,3,4-tetrahydronaphthalen-2- yl]benzenesulfonamide,
5-chloro-1,3-dimethyl-N-[(2R)-5-methyl-8-(4-methylpiperazin-1-yl)-1,2,3,4- tetrahydronaphthalen-2-yl]1H-pyrazole-4-sulfonamide,
3,5-dimethyl-N-[(2R)-5-methyl-8-(4-methylpiperazin-1-yl)-1,2,3,4-tetrahydronaphthalen-2- yl]isoxazole-4-sulfonamide,
2,6-difluoro-N-[(2R)-5-methyl-8-(4-methylpiperazin-1-yl)-1,2,3,4-tetrahydronaphthalen-2- yl]benzenesulfonamide,
2-chloro-4-fluoro-N-[(2R)-5-methyl-8-(4-methylpiperazin-1-yl)-1,2,3,4-tetrahydronaphthalen- 2-yl]benzenesulfonamide,
N-[(2R)-5-methyl-8-(4-methylpiperazin-1-yl)-1,2,3,4-tetrahydronaphthalen-2-yl]-2- (trifluoromethyl)benzenesulfonamide,
5-chloro-2-methoxy-N-[(2R)-5-methyl-8-(4-methylpiperazin-1-yl)-1,2,3,4- tetrahydronaphthalen-2-yl]benzenesulfonamide,
N-[(2R)-5-methyl-8-(4-methylpiperazin-1-yl)-1,2,3,4-tetrahydronaphthalen-2-yl]-4- nitrobenzenesulfonamide,
N-[(2R)-5-methyl-8-(4-methylpiperazin-1-yl)-1,2,3,4-tetrahydronaphthalen-2-yl]-1- phenylmethanesulfonamide,
4-fluoro-N-[(2R)-5-methyl-8-(4-methylpiperazin-1-yl)-1,2,3,4-tetrahydronaphthalen-2- yl]benzenesulfonamide,
N-[4-( {[(2R)-5-methyl-8-(4-methylpiperazin-1-yl)-1,2,3,4-tetrahydronaphthalen-2- yl]amino}sulfonyl)phenyl]acetamide,
2 ,5-difluoro-N-[(2R)-5-methyl-8-(4-methylpiperazin-1-yl)-1,2,3,4-tetrahydronaphthalen-2- yl]benzenesulfonamide,
methyl 1-methyl-5-( {[(2R)-5-methyl-8-(4-methylpiperazin-1-yl)-1,2,3,4- tetrahydronaphthalen-2-yl]amino}sulfonyl)-1H-pyrrole-2-carboxylate,
4-chloro-2-fluoro-N-[(2R)-5-methyl-8-(4-methylpiperazin-1-yl)-1,2,3,4-tetrahydronaphthalen- 2-yl]benzenesulfonamide,
3,5-dimethyl-N-[(2R)-5-methyl-8-(4-methylpiperazin-1-yl)-1,2,3,4-tetrahydronaphthalen-2- yl]benzenesulfonamide,
N-[(2R)-5-methyl-8-(4-methylpiperazin-1-yl)-1,2,3,4-tetrahydronaphthalen-2-yl]pyridine-3- sulfonamide,
5-bromo-2,4-difluoro-N-[(2R)-5-methyl-8-(4-methylpiperazin-1-yl)-1,2,3,4- tetrahydronaphthalen-2-yl]benzenesulfonamide,
5-bromo-6-chloro-N-[(2R)-5-methyl-8-(4-methylpiperazin-1-yl)-1,2,3,4- tetrahydronaphthalen-2-yl]pyridine-3-sulfonamide,
N-[(2R)-5-methyl-8-(4-methylpiperazin-1-yl)-1,2,3,4-tetrahydronaphthalen -2-yl]-1- benzothiophene-3-sulfonamide,
4-bromo-2,5-difluoro-N-[(2R)-5-methyl-8-(4-methylpiperazin-1-yl)-1,2,3,4- tetrahydronaphthalen-2-yl]benzenesulfonamide,
2,3 ,4-trifluoro-N-[(2R)-5-methyl-8-(4-methylpiperazin-1-yl)-1,2,3,4-tetrahydronaphthalen-2- yl]benzenesulfonamide,
N-[(2R)-5-methyl-8-(4-methylpiperazin-1-yl)-1,2,3,4-tetrahydronaphthalen-2-yl]thiophene-3- sulfonamide,
N-[(2R)-5-methyl-8-(4-methylpiperazin-1-yl)-1,2,3,4-tetrahydronaphthalen-2-yl]-4- (methylsulfonyl)benzenesulfonamide,
2-chloro-4-cyano-N-[(2R)-5-methyl-8-(4-methylpiperazin-1-yl)-1,2,3,4-tetrahydronaphthalen- 2-yl]benzenesulfonamide,
6-chloro-N-[(2R)-5-methyl-8-(4-methylpiperazin-1-yl)-1,2,3,4-tetrahydronaphthalen-2- yl]imidazo[2,1-b][1,3]thiazole-5-sulfonamide,
4-chloro-N-[(2R)-5-methyl-8-(4-methylpiperazin-1-yl)-1,2,3,4-tetrahydronaphthalen-2- yl]benzenesulfonamide,
3-chloro-N-[(2R)-5-methyl-8-(4-methylpiperazin-1-yl)-1,2,3,4-tetrahydronaphthalen-2- yl]benzenesulfonamide,
1-(3-chlorophenyl)-N-[(2R)-5-methyl-8-(4-methylpiperazin-1-yl)-1,2,3,4- tetrahydronaphthalen-2-yl]methanesulfonamide,
N-[4-methyl-5-( {[(2R)-5-methyl-8-(4-methylpiperazin-1-yl)-1,2,3,4-tetrahydronaphthalen-2- yl]amino}sulfonyl)-1,3-thiazol-2-yl]acetamide,
N-[(2R)-5-methyl-8-(4-methylpiperazin-1-yl)-1,2,3,4-tetrahydronaphthalen-2-yl]thiophene-2- sulfonamide,
N-[(2R)-5-methyl-8-(4-methylpiperazin-1-yl)-1,2,3,4-tetrahydronaphthalen-2-yl]-3- nitrobenzenesulfonamide,
2,5-dimethoxy-N-[(2R)-5-methyl-8-(4-methylpiperazin-1-yl)-1,2,3,4-tetrahydronaphthalen-2- yl]benzenesulfonamide,
5-bromo-2-methoxy-N-[(2R)-5-methyl-8-(4-methylpiperazin-1-yl)-1,2,3,4- tetrahydronaphthalen-2-yl]benzenesulfonamide,
N-[(2R)-5-methyl-8-(4-methylpiperazin-1-yl)-1,2,3,4-tetrahydronaphthalen-2-yl]-4- (phenylsulfonyl)thiophene-2-sulfonamide,
N-[(2R)-5-methyl-8-(4-methylpiperazin-1-yl)-1,2,3,4-tetrahydronaphthalen-2-yl]-5- (phenylsulfonyl)thiophene-2-sulfonamide,
N-[(2R)-5-methyl-8-(4-methylpiperazin-1-yl)-1,2,3,4-tetrahydronaphthalen-2-yl]-2,1,3- benzothiadiazole-4-sulfonamide,
N-[(2R)-5-methyl-8-(4-methylpiperazin-1-yl)-1,2,3,4-tetrahydronaphthalen-2-yl]-2,1,3- benzoxadiazole-4-sulfonamide,
5-isoxazol-3-yl-N-[(2R)-5-methyl-8-(4-methylpiperazin-1-yl)-1,2,3,4-tetrahydronaphthalen-2- yl]thiophene-2-sulfonamide,
2-methyl-N-[(2R)-5-methyl-8-(4-methylpiperazin- 1-yl)-1,2,3,4-tetrahydronaphthalen-2-yl]-5- nitrobenzenesulfonamide,
3-methyl-N-[(2R)-5-methyl-8-(4-methylpiperazin- 1-yl)-1,2,3,4-tetrahydronaphthalen-2- yl]benzenesulfonamide,
2-methyl-N-[(2R)-5-methyl-8-(4-methylpiperazin- 1-yl)-1,2,3,4-tetrahydronaphthalen-2-yl]-4- nitrobenzenesulfonamide,
3-chloro-4-fluoro-N-[(2R)-5-methyl-8-(4-methylpiperazin-1-yl)-1,2,3,4-tetrahydronaphthalen- 2-yl]benzenesulfonamide,
2,4-difluoro-N-[(2R)-5-methyl-8-(4-methylpiperazin-1-yl)-1,2,3,4-tetrahydronaphthalen-2- yl]benzenesulfonamide,
5-fluoro-2-methyl-N-[(2R)-5-methyl-8-(4-methylpiperazin- 1-yl)-1,2,3,4- tetrahydronaphthalen-2-yl]benzenesulfonamide,
1-[(IS,4R)-7,7-dimethyl-2-oxobicyclo[2.2.1]hept-1-yl]-N-[(2R)-5-methyl-8-(4- methylpiperazin-1-yl)-1,2,3,4-tetrahydronaphthalen-2-yl]methanesulfonamide,
2-fluoro-N-[(2R)-5-methyl-8-(4-methylpiperazin-1-yl)-1,2,3,4-tetrahydronaphthalen-2- yl]benzenesulfonamide,
3,4-dimethoxy-N-[(2R)-5-methyl-8-(4-methylpiperazin-1-yl)-1,2,3,4-tetrahydronaphthalen-2- yl]benzenesulfonamide,
5-chloro-N-[(2R)-5-methyl-8-(4-methylpiperazin-1-yl)-1,2,3,4-tetrahydronaphthalen-2- yl]thiophene-2-sulfonamide,
2-chloro-6-methyl-N-[(2R)-5-methyl-8-(4-methylpiperazin-1-yl)-1,2,3,4- tetrahydronaphthalen-2-yl]benzenesulfonamide,
5-bromo-N-[(2R)-5-methyl-8-(4-methylpiperazin-1-yl)-1,2,3,4-tetrahydronaphthalen-2- yl]thiophene-2-sulfonamide,
4-chloro-N-[(2R)-5-methyl-8-(4-methylpiperazin-1-yl)-1,2,3,4-tetrahydronaphthalen-2-yl]-3- nitrobenzenesulfonamide,
3-fluoro-N-[(2R)-5-methyl-8-(4-methylpiperazin-1-yl)-1,2,3,4-tetrahydronaphthalen-2- yl]benzenesulfonamide,
N-[2-chloro-4-( {[(2R)-5-methyl-8-(4-methylpiperazin-1-yl)-1,2,3,4-tetrahydronaphthalen-2- yl]amino}sulfonyl)phenyl]acetamide,
2-methoxy-5-methyl-N-[(2R)-5-methyl-8-(4-methylpiperazin-1-yl)-1,2,3,4- tetrahydronaphthalen-2-yl]benzenesulfonamide,
N-[(2R)-5-methyl-8-(4-methylpiperazin-1-yl)-1,2,3,4-tetrahydronaphthalen-2-yl]-2- nitrobenzenesulfonamide,
3,4,5-trimethoxy-N-[(2R)-5-methyl-8-(4-methylpiperazin-1-yl)-1,2,3,4-tetrahydronaphthalen- 2-yl]benzenesulfonamide,
4-bromo-N-[(2R)-5-methyl-8-(4-methylpiperazin-1-yl)-1,2,3,4-tetrahydronaphthalen-2- yl]benzenesulfonamide,
2-bromo-N-[(2R)-5-methyl-8-(4-methylpiperazin-1-yl)-1,2,3,4-tetrahydronaphthalen-2- yl]benzenesulfonamide,
N-[(2R)-5-methyl-8-(4-methylpiperazin-1-yl)-1,2,3,4-tetrahydronaphthalen-2-yl]-4- (trifluoromethyl)benzenesulfonamide,
4-ethyl-N-[(2R)-5-methyl-8-(4-methylpiperazin-1-yl)-1,2,3,4-tetrahydronaphthalen-2- yl]benzenesulfonamide,
N-[(2R)-5-methyl-8-(4-methylpiperazin-1-yl)-1,2,3,4-tetrahydronaphthalen-2-yl]-2-nitro-4- (trifluoromethyl)benzenesulfonamide,
4-methyl-N-[(2R)-5-methyl-8-(4-methylpiperazin-1-yl)-1,2,3,4-tetrahydronaphthalen-2-yl]-3- nitrobenzenesulfonamide,
N-[(2R)-5-methyl-8-(4-methylpiperazin-1-yl)-1,2,3,4-tetrahydronaphthalen-2-yl]naphthalene- 1-sulfonamide,
N-[(2R)-5-methyl-8-(4-methylpiperazin-1-yl)-1,2,3,4-tetrahydronaphthalen-2-yl]-2-oxo- 1,2,3,4-tetrahydroquinoline-6-sulfonamide,
1-[(1R,4S)-7,7-dimethyl-2-oxobicyclo[2.2.1]hept-1-yl]-N-[(2R)-5-methyl-8-(4- methylpiperazin-1-yl)-1,2,3,4-tetrahydronaphthalen-2-yl]methanesulfonamide,
3,4-difluoro-N-[(2R)-5-methyl-8-(4-methylpiperazin-1-yl)-1,2,3,4-tetrahydronaphthalen-2- yl]benzenesulfonamide,
2-methyl-N-[(2R)-5-methyl-8-(4-methylpiperazin-1-yl)-1,2,3,4-tetrahydronaphthalen-2- yl]benzenesulfonamide,
2,5-dimethyl-N-[(2R)-5-methyl-8-(4-methylpiperazin-1-yl)-1,2,3,4-tetrahydronaphthalen-2- yl]benzenesulfonamide,
N-[(2R)-5-methyl-8-(4-methylpiperazin-1-yl)-1,2,3,4-tetrahydronaphthalen-2-yl]-4-(pyridin- 3-yloxy)benzenesulfonamide,
4-chloro-2,5-dimethoxy-N-[(2R)-5-methyl-8-(4-methylpiperazin-1-yl)-1,2,3,4- tetrahydronaphthalen-2-yl]benzenesulfonamide,
6-chloro-N-[(2R)-5-methyl-8-(4-methylpiperazin-1-yl)-1,2,3,4-tetrahydronaphthalen-2-yl]- 2H-1,2,4-benzothiadiazine-7-sulfonamide 1,1-dioxide,
methyl 2-methyl-5-({[(2R)-⁵-methyl-8-(4-methylpiperazin-1-yl)-1,2,3,4- tetrahydronaphthalen-2-yl]amino}sulfonyl)-3-furoate,
2-methoxy-5-( {[(2R)-5-methyl-8-(4-methylpiperazin-1-yl)-1,2,3,4-tetrahydronaphthalen-2- yl]amino}sulfonyl)benzamide,
3-cyano-4-fluoro-N-[(2R)-5-methyl-8-(4-methylpiperazin-1-yl)-1,2,3,4-tetrahydronaphthalen- 2-yl]benzenesulfonamide,
3-fluoro-4-methyl-N-[(2R)--methyl-8-(4-methylpiperazin-1-yl)-1,2,3,4- tetrahydronaphthalen-2-yl]benzenesulfonamide,
4-fluoro-2-methyl-N-[(2R)-5-methy-8-(4-methylpiperazin-1-yl)-1,2,3,4- tetrahydronaphthalen-2-yl]benzenesulfonamide,
2-methoxy-N-[(2R)-5-methyl-8-(4-methylpiperazin-1-yl)-1,2,3,4-tetrahydronaphthalen-2-yl]- 4-nitrobenzenesulfonamide,
2,4,5-trifluoro-N-[(2R)--methyl-8-(4-methylpiperazin-1-yl)-1,2,3,4-tetrahydronaphthalen-2- yl]benzenesulfonamide,
methyl 3-[4-( {[(2R)-5-methyl-8-(4-methylpiperazin-1-yl)-1,2,3,4-tetrahydronaphthalen-2- yl]amino}sulfonyl)phenyl]propanoate,
N-[(2R)-5-methyl-8-(4-methylpiperazin- 1-yl)-1,2,3,4-tetrahydronaphthalen-2-yl]-6- phenoxypyridine-3-sulfonamide,
N-[(2R)-5-methyl-8-(4-methylpiperazin-1-yl)-1,2,3,4-tetrahydronaphthalen-2-yl]-2,3-dihydro- 1,4-benzodioxine-6-sulfonamide,
N-[(2R)-5-methyl-8-(4-methylpiperazin-1-yl)-1,2,3,4-tetrahydronaphthalen-2-yl]-1- benzofuran-2-sulfonamide,
4-chloro-N1-[(2R)--methyl-8-(4-methylpiperazin-1-yl)-1,2,3,4-tetrahydronaphthalen-2- yl]benzene-1,3-disulfonamide,
4-fluoro-N-[(2R)-5-methyl-8-(4-methylpiperazin-1-yl)-1,2,3,4-tetrahydronaphthalen-2- yl]naphthalene- 1-sulfonamide,
3,5-difluoro-N-[(2R)-5-methyl-8-(4-methylpiperazin-1-yl)-1,2,3,4-tetrahydronaphthalen-2- yl]benzenesulfonamide,
4-fluoro-N-[(2R)-5-methyl-8-(4-methylpiperazin-1-yl)-1,2,3,4-tetrahydronaphthalen-2-yl]-3- (trifluoromethyl)benzenesulfonamide,
2-chloro-4,5-difluoro-N-[(2R)-5-methyl-8-(4-methylpiperazin-1-yl)-1,2,3,4- tetrahydronaphthalen-2-yl]benzenesulfonamide,
5-chloro-2,4-difluoro-N-[(2R)-5-methyl-8-(4-methylpiperazin-1-yl)-1,2,3,4- tetrahydronaphthalen-2-yl]benzenesulfonamide,
4-chloro-2,5-difluoro-N-[(2R)-5-methyl-8-(4-methylpiperazin- 1-yl)-1,2,3,4- tetrahydronaphthalen-2-yl]benzenesulfonamide,
3 -chloro-4-methyl-N-[(2R)-5-methyl-8-(4-methylpiperazin-1-yl)-1,2,3,4- tetrahydronaphthalen-2-yl]benzenesulfonamide,
N-[(2R)-5-methyl-8-(4-methylpiperazin-1-yl)-1,2,3,4-tetrahydronaphthalen-2-yl]-2- (methylsulfonyl)benzenesulfonamide,
1,3,5-trimethyl-N-[(2R)-5-methyl-8-(4-methylpiperazin-1-yl)-1,2,3,4-tetrahydronaphthalen-2- yl]1 H-pyrazole-4-sulfonamide,
N-[(2R)-5-methyl-8-(4-methylpiperazin-1-yl)-1,2,3,4-tetrahydronaphthalen-2-yl]-1-(3- nitrophenyl)methanesulfonamide,
5-methyl-N-[(2R)-5-methyl-8-(4-methylpiperazin-1-yl)-1,2,3,4-tetrahydronaphthalen-2-yl]- 2,1,3-benzothiadiazole-4-sulfonamide,
2,5-dimethoxy-N-[(2R)-5-methyl-8-(4-methylpiperazin-1-yl)-1,2,3,4-tetrahydronaphthalen-2- yl]-4-nitrobenzenesulfonamide,
N-[(2R)-5-methyl-8-(4-methylpiperazin-1-yl)-1,2,3,4-tetrahydronaphthalen-2-yl]-1-oxo-1,2- dihydroisoquinoline-4-sulfonamide,
dimethyl 5-( {[(2R)-5-methyl-8-(4-methylpiperazin-1-yl)-1,2,3,4-tetrahydronaphthalen-2- yl]amino}sulfonyl)isophthalate,
4-methyl-N-[(2R)-5-methyl-8-(4-methylpiperazin-1-yl)-1,2,3,4-tetrahydronaphthalen-2- yl]benzenesulfonamide,
4-methoxy-N-[(2R)-5-methyl-8-(4-methylpiperazin-1-yl)-1,2,3,4-tetrahydronaphthalen-2- yl]benzenesulfonamide,
2-chloro-N-[(2R)-5-methyl-8-(4-methylpiperazin-1-yl)-1,2,3,4-tetrahydronaphthalen-2- yl]benzenesulfonamide,
N-[(2R)-5-methyl-8-(4-methylpiperazin-1-yl)-1,2,3,4-tetrahydronaphthalen-2-yl]-3- (trifluoromethyl)benzenesulfonamide,
N-[(2R)-5-methyl-8-(4-methylpiperazin-1-yl)-1,2,3,4-tetrahydronaphthalen-2-yl]-5-pyridin-2- ylthiophene-2-sulfonamide,
2-cyano-N-[(2S)-5-methyl-8-(4-methylpiperazin-1-yl)-1,2,3,4-tetrahydronaphthalen-2- yl]benzenesulfonamide,
4-bromo-2-fluoro-N-[(2R)-5-methyl-8-(4-methylpiperazin-1-yl)-1,2,3,4-tetrahydronaphthalen- 2-yl]benzenesulfonamide,
N-[3-({[(2R)-5-methyl-8-(4-methylpiperazin-1-yl)-1,2,3,4-tetrahydronaphthalen-2- yl]amino}sulfonyl)phenyl]acetamide,
3-chloro-2-methyl-N-[(2R)-5-methyl-8-(4-methylpiperazin-1-yl)-1,2,3,4- tetrahydronaphthalen-2-yl]benzenesulfonamide,
4-methoxy-N-[(2R)-5-methyl-8-(4-methylpiperazin-1-yl)-1,2,3,4-tetrahydronaphthalen-2-yl]- 2-nitrobenzenesulfonamide,
3-methoxy-N-[(2R)-5-methyl-8-(4-methylpiperazin-1-yl)-1,2,3,4-tetrahydronaphthalen-2- yl]benzenesulfonamide,
N-[(2R)-5-methyl-8-(4-methylpiperazin-1-yl)-1,2,3,4-tetrahydronaphthalen-2-yl]-2- (phenylsulfonyl)benzenesulfonamide,
(E)-N-[(2R)-5-methyl-8-(4-methylpiperazin-1-yl)-1,2,3,4-tetrahydronaphthalen-2-yl]-2- phenylethylenesulfonamide,
2-methoxy-4-methyl-N-[(2R)-5-methyl-8-(4-methylpiperazin-1-yl)-1,2,3,4- tetrahydronaphthalen-2-yl]benzenesulfonamide,
3-(difluoromethoxy)-N-[(2R)-5-methyl-8-(4-methylpiperazin-1-yl)-1,2,3,4- tetrahydronaphthalen-2-yl]benzenesulfonamide,
4-bromo-3-fluoro-N-[(2R)-5-methyl-8-(4-methylpiperazin-1-yl)-1,2,3,4-tetrahydronaphthalen- 2-yl]benzenesulfonamide,
N-[(2R)-5-methyl-8-(4-methylpiperazin-1-yl)-1,2,3,4-tetrahydronaphthalen-2-yl]-5-(1,2,3- thiadiazol-4-yl)thiophene-2-sulfonamide, and
methyl 2,5-dimethyl-4-({[(2S)-5-methyl-8-(4-methylpiperazin-1-yl)-1,2,3,4- tetrahydronaphthalen-2-yl]amino}sulfonyl)-3-furoate,
or pharmaceutically acceptable salts, solvates or solvated salts thereof.

28. A compound according to claim 19, for use in therapy.

29. Use of a compound according to claim 19, in the manufacture of a medicament for treatment of 5-HT6 mediated disorders.

30. A pharmaceutical composition comprising as active ingredient a therapeutically effective amount of a compound according to claim 19, in association with one or more pharmaceutically acceptable diluents, excipients or inert carriers.

31. A method of treatment of Alzheimer's disease, cognitive impairment associated with schizophrenia, obesity or Parkinson's disease, comprising administering to a mammal, including man in need of such treatment, a therapeutically effective amount of a pharmaceutical composition according to claim 30.

32. A method of treatment of Alzheimer's disease, cognitive impairment associated with schizophrenia, obesity or Parkinson's disease, comprising administering to a mammal, including man in need of such treatment, a therapeutically effective amount of a compound, according to claim 19.

33. An agent for the prevention or treatment of Alzheimer's disease, cognitive impairment associated with schizophrenia, obesity or Parkinson's disease, which comprises as active ingredient a compound, according to claim 19.
